(19) United States
(12) Patent Application Publication
(10) Pub. No.: US 2009/0270379 A1
(43) Pub. Date: Oct. 29, 2009

(54) QUINOLONE DERIVATIVES USEFUL AS ANTIBACTERIAL AGENTS

(76) Inventors: Mark J. Macielag, Branchburg, NJ (US); Bin Zhu, Hillsborough, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/426,442

(22) Filed: Apr. 20, 2009

Related U.S. Application Data
(60) Provisional application No. 61/047,124, filed on Apr. 23, 2008.

(51) Int. Cl. A61K 31/5383; C07D 487/04; A61K 31/498; C07D 498/06; A61P 31/04

(52) U.S. Cl. 514/230.2; 544/349; 514/249; 544/101

(57) ABSTRACT

The present invention is directed to quinolone derivatives, useful as antimicrobial compounds, pharmaceutical compositions comprising said derivatives and the use of said derivatives and pharmaceutical compositions as antimicrobial agents against pathogenic microorganisms, particularly against resistant microbes

### CROSS REFERENCE TO RELATED APPLICATIONS

This application claims priority of the benefits of the filing of U.S. Provisional Application Ser. No. 61/047,124, filed Apr. 23, 2008. The complete disclosures of the aforementioned related U.S. patent application is hereby incorporated herein by reference for all purposes.

### FIELD OF INVENTION

The present invention is directed to quinolone derivatives, useful as antimicrobial compounds, pharmaceutical compositions comprising said derivatives and the use of said derivatives and pharmaceutical compositions as antimicrobial agents against pathogenic microorganisms, particularly against resistant microbes.

### BACKGROUND OF THE INVENTION

The chemical and medical literature describes compounds that are said to be antimicrobial, i.e., capable of destroying or suppressing the growth or reproduction of microorganisms, such as bacteria. For example, such antibacterial agents are described in Antibiotics, Chemotherapeutics, and Antibacterial Agents for Disease Control (M. Greyson, editor, 1982), E. Gale et al., The Molecular Basis of Antibiotic Action 2d edition (1981), Recent Research Developments in Antimicrobial Agents & Chemotherapy (S. G. Pandalai, Editor, 2001), Quinolone Antimicrobial Agents (John S Wolfson, David C Hooper, Editors, 1989), and F. O'Grady, H. P. Lambert, R. G. Finch, D. Greenwood, Martin Dedicoat, “Antibiotic and Chemotherapy, 7th edn.” (1997).

The mechanisms of action of these antibacterial agents vary. However, they are generally believed to function in one or more ways: by inhibiting cell wall synthesis or repair; by altering cell wall permeability; by inhibiting protein synthesis; or by inhibiting the synthesis of nucleic acids. For example, beta-lactam antibacterial agents act through inhibiting essential penicillin binding proteins (PBPs) in bacteria, which are responsible for cell wall synthesis. As another example, quinolones act, at least in part by inhibiting synthesis of DNA, thus preventing the cell from replicating.

The pharmacological characteristics of antimicrobial agents, and their suitability for any given clinical use, vary. For example, the classes of antimicrobial agents (and members within a class) may vary in 1) their relative efficacy against different types of microorganisms, 2) their susceptibility to development of microbial resistance and 3) their pharmacological characteristics such as their bioavailability and biodistribution. Accordingly, selection of an appropriate antimicrobial agent in a given clinical situation requires analysis of many factors, including the type of organism involved, the desired method of administration, the location of the infection to be treated and other considerations.

However, many such attempts to produce improved antimicrobial agents yield equivocal results. Indeed, few antimicrobial agents are produced that are truly clinically acceptable in terms of their spectrum of antimicrobial activity, avoidance of microbial resistance, and pharmacology. Thus there is a continuing need for broad-spectrum antimicrobial agents, which are effective against resistant microbes.

Examples of bacterial infections resistant to antibiotic therapy have been reported in the past; they are now a significant threat to public health in the developed world. The development of microbial resistance (perhaps as a result of the intense use of antibacterial agents over extended periods of time) is of increasing concern in medical science. “Resistance” can be defined as existence of organisms, within a population of a given microbial species, that are less susceptible to the action of a given antimicrobial agent. This resistance is of particular concern in environments such as hospitals and nursing homes, where relatively high rates of infection and intense use of antibacterial agents are common. See, e.g., W. Sanders, Jr. et al., “Inducible Beta-lactamases: Clinical and Epidemiologic Implications for the Use of Newer Cephalosporins”, Review of Infectious Diseases, p. 830 (1988).

Pathogenic bacteria are known to acquire resistance via several distinct mechanisms including inactivation of the antibiotic by bacterial enzymes (e.g., β-lactamases hydrolyzing penicillin and cephalosporins); removal of the antibiotic using efflux pumps; modification of the target of the antibiotic via mutation and genetic recombination (e.g., penicillin-resistance in Neiserria gonorrhoeae); and acquisition of a readily transferable gene from an external source to create a resistant target (e.g., methicillin-resistance in Staphylococcus aureus). There are certain Gram-positive pathogens, such as vancomycin-resistant Enterococcus faecium, which are resistant to most commercially available antibiotics.

Hence existing antibacterial agents have limited capacity in overcoming the threat of resistance. Thus it would be advantageous to provide new antibacterial agents that can be used against resistant microbes.

### SUMMARY OF INVENTION

The present invention is directed to compounds of formula (I)

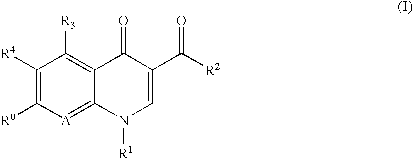

wherein

A is selected from the group consisting of N and CR²⁰; wherein R²⁰ is selected from the group consisting of hydrogen, fluoro, chloro, hydroxy, C₁₋₄alkyl, halogenated C₁₋₄alkyl, C₁₋₄alkoxy, halogenated C₁₋₄alkoxy, C₁-C₄alkylthio, amino, (C₁₋₄alkyl)amino, di(C₁₋₄alkyl)amino and cyano;

alternatively, A is CR²⁰, and R¹ and R²⁰ are taken together with the atoms to which they are bound to form

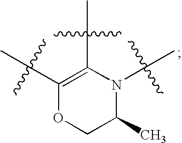

R¹ is selected from the group consisting of C₃-C₆cycloalkyl, C₄-C₆heterocycloalkyl, C₁-C₈alkyl, C₂-C₈alkenyl, phenyl and a 5 to 6 membered heteroaryl; wherein the C₃-C₆cycloalkyl, C₄-C₆heterocycloalkyl, phenyl or 5 to 6 membered heteroaryl is optionally substituted with one or more substituents independently selected from the group consisting of fluoro, chloro, C₁₋₄alkyl, C₁₋₄alkoxy, cyano, nitro, amino, (C₁₋₄alkyl)amino and di(C₁₋₄alkyl)amino;

R² is selected from the group consisting of hydroxy, C₁₋₄alkoxy and benzyloxy;

R³ and R⁴ are each independently selected from the group consisting of hydrogen, fluoro, chloro, hydroxy, amino, (C₁₋₄alkyl)amino, di(C₁₋₄alkyl)amino, C₁₋₄alkyl, halogenated C₁₋₄alkyl, C₁₋₄alkoxy, halogenated C₁₋₄alkoxy and C₁-C₄alkylthio;

R⁰ is selected from the group consisting of

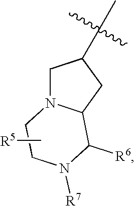

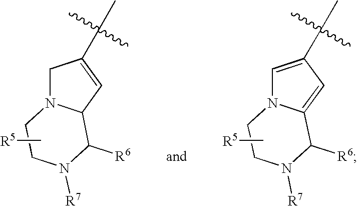

R⁵ and R⁶ are each independently selected from the group consisting of hydrogen, C₁₋₄alkyl, hydroxy substituted C₁₋₄alkyl, halogenated C₁₋₄alkyl, oxo, C₃₋₈cycloalkyl and phenyl; provided that when R⁶ is oxo and R⁵ is oxo, then R⁵ is bound to the carbon atom which is alpha to the nitrogen of the ring fusion (i.e. alpha to the pyrrolidine nitrogen);

R⁷is selected from the group consisting hydrogen, C₁₋₄alkyl, C₃₋₈cycloalkyl, —(C₁₋₄alkyl)-C₃₋₈cycloalkyl, aryl, aralkyl, heteroaryl, —(C₁₋₄alkyl)-heteroaryl, heterocycloalkyl, —(C₁₋₄alkyl)-heterocycloalkyl, —C(O)—R⁸, —C(O)O—R⁸, —C(O)—NR⁹R¹⁰ and —C(O)—(C₁₋₄alkyl)-NR⁹R¹⁰;

wherein the C₁₋₄alkyl is optionally substituted with one or more substituents independently selected from the group consisting of halogen, hydroxy, oxo, thio, cyano, —NR¹¹R¹², aryloxy, heteroaryloxy, acyloxy, carboxy, carboxamido and acylamino;

wherein the C₃₋₈cycloalkyl, whether alone or as part of a substituent group is optionally substituted with one or more fluoro;

and wherein the aryl, heteroaryl or heterocycloalkyl, whether alone or as part of a substituent group, is optionally substituted with one to three substituents independently selected from the group consisting of halogen, hydroxy, oxo, cyano, thio, nitro, —NR¹³R¹⁴, C₁-C₈alkyl, halogenated C₁-C₈alkyl, C₁-C₈alkoxy, halogenated C₁-C₈alkoxy, C₁-C₈alkylthio, formyl, carboxy, —C(O)O—(C₁₋₄alkyl), —O—C(O)—(C₁-C₄alkyl), —NR¹⁵—C(O)—(C₁-C₄alkyl) and —C(O)—NR¹⁶R¹⁷;

R⁸ is selected from the group consisting of C₁₋₄alkyl;

R⁹ and R¹⁰ are each independently selected from the group consisting of hydrogen and C₁₋₄alkyl; alternatively, R⁹ and R¹⁰ are taken together with the nitrogen atom to which they are bound to form a 5 to 6 membered nitrogen containing saturated ring structure;

R¹¹ and R¹² are each independently selected from the group consisting of hydrogen and C₁₋₄alkyl; alternatively, R¹¹ and R¹² are taken together with the nitrogen atom to which they are bound to form a 5 to 6 membered nitrogen containing saturated ring structure;

R¹³ and R¹⁴ are each independently selected from the group consisting of hydrogen and C₁₋₄alkyl; alternatively, R¹³ and R¹⁴ are taken together with the nitrogen atom to which they are bound to form a 5 to 6 membered nitrogen containing saturated ring structure;

R¹⁵ is selected from the group consisting of hydrogen and C₁₋₄alkyl;

R¹⁶ and R¹⁷ are each independently selected from the group consisting of hydrogen and C₁₋₄alkyl; alternatively, R¹⁶ and R¹⁷ are taken together with the nitrogen atom to which they are bound to form a 5 to 6 membered nitrogen containing saturated ring structure;

and optical isomers, enantiomers, diastereomers, racemates or racemic mixture thereof, and pharmaceutically acceptable salts, hydrates, esters or prodrugs thereof.

Illustrative of the invention is a pharmaceutical composition comprising a pharmaceutically acceptable carrier and the product prepared according to the process described herein. An illustration of the invention is a pharmaceutical composition made by mixing the product prepared according to the process described herein and a pharmaceutically acceptable carrier. Illustrating the invention is a process for making a pharmaceutical composition comprising mixing the product prepared according to the process described herein and a pharmaceutically acceptable carrier.

It has been found that the compounds of this invention, and compositions containing these compounds, are effective antimicrobial agents against a broad range of pathogenic microorganisms with advantages of activity against resistant microbes.

Accordingly, the present invention is also directed to a method of treating a subject having a condition caused by or contributed to by bacterial infection, which comprises administering to said mammal a therapeutically effective amount of the compound of Formula (I).

The present invention is further directed to a method of preventing a subject from suffering from a condition caused by or contributed to by bacterial infection, which comprises administering to the subject a prophylactically effective dose of the pharmaceutical composition of a compound of Formula (I).

The present invention is further directed to the use of a compound of formula (I) for the preparation of a medicament for treating and/or preventing a condition caused by or contributed to by bacterial infection, in a subject in need thereof. In an embodiment, the present invention is directed to the use of a compound of formula (I) for the preparation of a medicament for treating and/or preventing a condition caused by or contributed to by bacterial infection associated with a drug resistant bacteria, in a subject in need thereof.

### DETAILED DESCRIPTION OF THE INVENTION

The present invention is directed to compounds of formula (I)

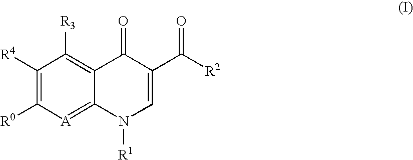

wherein R⁰, R¹, R², R³, R⁴ and A are a herein defined, and optical isomers, diastereomers, enantiomers, pharmaceutically acceptable salts, hydrates, and prodrugs thereof. The compounds of formula (I) are useful as antimicrobial agents against pathogenic microorganisms, preferably, resistant microbes. Compounds of formula (I) wherein R⁰ is

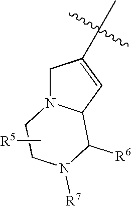

are also useful as intermediates in the synthesis of compounds of formula (I) wherein R⁰ is selected from the group consisting of

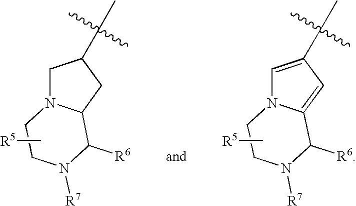

In an embodiment, the present invention is directed to compounds of formula (I-A)

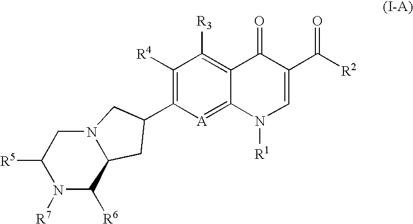

and optical isomers, diastereomers, enantiomers, pharmaceutically acceptable salts, hydrates, and prodrugs thereof. In another embodiment, the present invention is directed to compounds of formula (I-B)

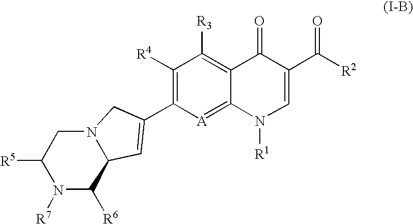

and optical isomers, diastereomers, enantiomers, pharmaceutically acceptable salts, hydrates, and prodrugs thereof. In another embodiment, the present invention is directed to compounds of formula (I-C)

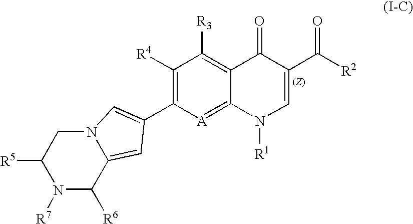

and optical isomers, diastereomers, enantiomers, pharmaceutically acceptable salts, hydrates, and prodrugs thereof. In yet another embodiment, the present invention is directed to compounds of formula (I) wherein A is CR²⁰ and wherein R²⁰ and R¹ are taken together with the atoms to which they are bound to form

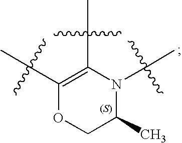

compounds of formula (I-D)

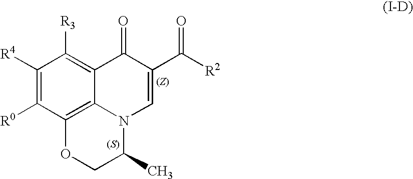

and optical isomers, diastereomers, enantiomers, pharmaceutically acceptable salts, hydrates, and prodrugs thereof.

In an embodiment of the present invention, A is selected from the group N and CR²⁰; wherein R²⁰ is selected from the group consisting of hydrogen, fluoro, chloro, hydroxy, C₁₋₄alkyl, fluorinated C₁₋₄alkyl, C₁₋₄alkoxy and fluorinated C₁₋₂alkoxy; alternatively, A is CR²⁰, and R²⁰ and R¹ are taken together with the atoms to which they are bound to form

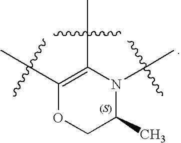

In another embodiment of the present invention, A is selected from the group N and CR²⁰; wherein R²⁰ is selected from the group consisting of hydrogen, C₁₋₂alkoxy and fluorinated C₁₋₂alkoxy; alternatively, A is CR²⁰, and R²⁰ and R¹ are taken together with the atoms to which they are bound to form

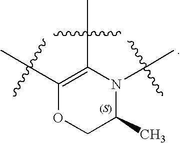

In another embodiment of the present invention, A is selected from the group N, CH, C—OCH₃ and C—OCHF₂; alternatively, A is CR²⁰, and R²⁰ and R¹ are taken together with the atoms to which they are bound to form

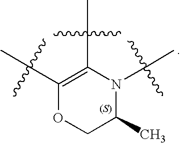

In an embodiment of the present invention, R¹ is selected from the group consisting of C₃-C₆cycloalkyl, C₄-C₆heterocycloalkyl, phenyl and a 5 to 6 membered heteroaryl; wherein the C₃-C₆cycloalkyl, C₄-C₆heterocycloalkyl, phenyl or 5 to 6 membered heteroaryl is optionally substituted with one to two substituents independently selected from the group consisting of fluoro, chloro, C₁₋₄alkyl, C₁₋₄alkoxy, cyano, nitro, amino, (C₁₋₄alkyl)amino and di(C₁₋₄alkyl)amino. In another embodiment of the present invention, R¹ is selected from the group consisting of C₃₋₆cycloalkyl and phenyl; wherein the phenyl is optionally substituted with one to two halogen. In another embodiment of the present invention, R¹ is selected from the group consisting of cyclopropyl and 2,4-difluorophenyl.

In an embodiment of the present invention, R² is selected from the group consisting of hydroxy, C₁₋₄alkoxy and benzyloxy. In another embodiment of the present invention, R² is selected from the group consisting of hydroxy and C₁₋₄alkoxy. In another embodiment of the present invention, R² is hydroxy.

In an embodiment of the present invention, R³ and R⁴ are each independently selected from the group consisting of hydrogen, fluoro, chloro, hydroxy, C₁₋₄alkyl, fluorinated C₁₋₄alkyl, C₁₋₄alkoxy and fluorinated C₁₋₄alkoxy. In another embodiment of the present invention, R³ and R⁴ are each independently selected from the group consisting of hydrogen and fluoro.

In an embodiment of the present invention, R³ is hydrogen. In an embodiment of the present invention, R⁴ is selected from the group consisting of hydrogen and fluoro.

In an embodiment of the present invention, R⁰ is selected from the group consisting of

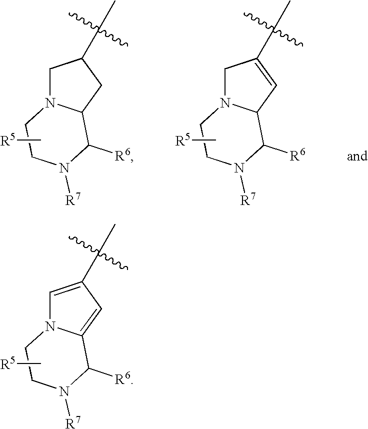

In another embodiment of the present invention, R⁰ is selected from the group consisting of

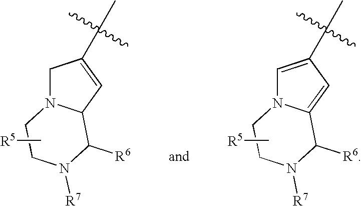

In another embodiment of the present invention, R⁰ is

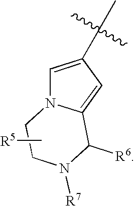

In an embodiment of the present invention, R⁵ and R⁶ are each independently selected from the group consisting of hydrogen, C₁₋₄alkyl, hydroxy substituted C₁₋₄alkyl, halogenated C₁₋₄alkyl and oxo; provided that when R⁶ is oxo and R⁵ is oxo, then R⁵ is bound to the carbon atom which is alpha to the nitrogen of the ring fusion. In another embodiment of the present invention, R⁵ and R⁶ are each independently selected from the group consisting of hydrogen, C₁₋₄alkyl and hydroxy substituted C₁₋₄alkyl. In another embodiment of the present invention, R⁵ and R⁶ are each independently selected from the group consisting of hydrogen, methyl and hydroxymethyl.

In an embodiment of the present invention, R⁵ is bound at the carbon atom, which is alpha to the N—R⁷ portion of the R⁰ substituent group. In another embodiment of the present invention, R⁵ is bound at the carbon atom, which is beta to the N—R⁷ portion of the R⁰ substituent group.

In an embodiment of the present invention, R⁵ is selected from the group consisting of hydrogen, methyl and hydroxymethyl; and wherein R⁵ is bound to the piperazinyl portion of R⁰ at the carbon atom, which is alpha to the N—R⁷ portion of R⁰. In an embodiment of the present invention, R⁶ is selected from the group consisting of hydrogen and methyl.

In an embodiment of the present invention, R⁷ is selected from the group consisting hydrogen, C₁₋₄alkyl, —(C₁₋₄alkyl)-C₃₋₈cycloalkyl, aralkyl, —(C₁₋₄alkyl)-heteroaryl, —(C₁₋₄alkyl)-heterocycloalkyl, —C(O)—R⁸ and —C(O)—(C₁₋₄alkyl)-NR⁹R¹⁰; wherein R⁸ is selected from the group consisting of C₁₋₄alkyl; and wherein R⁹ and R¹⁰ are each independently selected from the group consisting of hydrogen and C₁₋₄alkyl; alternatively, R⁹ and R¹⁰ are taken together with the nitrogen atom to which they are bound to form a 5 to 6 membered nitrogen containing saturated ring structure. In another embodiment of the present invention, R⁷ is selected from the group consisting of hydrogen, C₁₋₄alkyl, aralkyl and —C(O)—(C₁₋₄alkyl)-NR⁹R¹⁰; wherein R⁹ and R¹⁰ are each independently selected form the group consisting of hydrogen and C₁₋₄alkyl. In another embodiment of the present invention, R⁷ is selected from the group consisting of hydrogen, methyl, benzyl and (S)-2-(methylamino)propionyl. In another embodiment of the present invention, R⁷ is selected from the group consisting of hydrogen, methyl and (S)-2-(methylamino)propionyl.

In an embodiment of the present invention, the compound of formula (I) is selected from the group consisting of 1-Cyclopropyl-8-difluoromethoxy-4-oxo-7-(1,2,3,4-tetrahydro-pyrrolo[1,2-a]pyrazin-7-yl)-1,4-dihydro-quinoline-3-carboxylic acid; 7-(2-Benzyl-1,2,3,4,6,8a-hexahydro-pyrrolo[1,2-a]pyrazin-7-yl)-1-cyclopropyl-8-difluoromethoxy-4-oxo-1,4-dihydro-quinoline-3-carboxylic acid; 1-Cyclopropyl-8-difluoromethoxy-7-(octahydro-pyrrolo[1,2-a]pyrazin-7-yl)-4-oxo-1,4-dihydro-quinoline-3-carboxylic acid; 1-Cyclopropyl-8-difluoromethoxy-7-(2-methyl-1,2,3,4-tetrahydro-pyrrolo[1,2-a]pyrazin-7-yl)-4-oxo-1,4-dihydro-quinoline-3-carboxylic acid; 1-Cyclopropyl-8-difluoromethoxy-7-(2-methyl-octahydro-pyrrolo[1,2-a]pyrazin-7-yl)-4-oxo-1,4-dihydro-quinoline-3-carboxylic acid; 1-Cyclopropyl-8-difluoromethoxy-7-(3S-methyl-octahydro-pyrrolo[1,2-a]pyrazin-7-yl)-4-oxo-1,4-dihydro-quinoline-3-carboxylic acid; 1-Cyclopropyl-8-difluoromethoxy-7-(3S-methyl-1,2,3,4-tetrahydro-pyrrolo[1,2-a]pyrazin-7-yl)-4-oxo-1,4-dihydro-quinoline-3-carboxylic acid; 1-Cyclopropyl-8-difluoromethoxy-7-(1S-methyl-1,2,3,4-tetrahydro-pyrrolo[1,2-a]pyrazin-7-yl)-4-oxo-1,4-dihydro-quinoine-3-carboxyic acid; 1-Cyclopropyl-8-difluoromethoxy-7-(1R-methyl-1,2,3,4-tetrahydro-pyrrolo[1,2-a]pyrazin-7-yl)-4-oxo-1,4-dihydro-quinoline-3-carboxylic acid; 1-Cyclopropyl-8-difluoromethoxy-7-[2S-(2-methylamino-propionyl)-1,2,3,4-tetrahydro-pyrrolo[1,2-a]pyrazin-7-yl]-4-oxo-1,4-dihydro-quinoline-3-carboxylic acid; 1-Cyclopropyl-6-fluoro-8-methoxy-7-(3S-methyl-1,2,3,4-tetrahydro-pyrrolo[1,2-a]pyrazin-7-yl)-4-oxo-1,4-dihydro-quinoline-3-carboxylic acid; 1-Cyclopropyl-6-fluoro-8-methoxy-7-(3R-methyl-1,2,3,4-tetrahydro-pyrrolo[1,2-a]pyrazin-7-yl)-4-oxo-1,4-dihydro-quinoline-3-carboxylic acid; 1-Cyclopropyl-7-(2,3S-dimethyl-1,2,3,4-tetrahydro-pyrrolo[1,2-a]pyrazin-7-yl)-6-fluoro-8-methoxy-4-oxo-1,4-dihydro-quinoline-3-carboxylic acid; 1-Cyclopropyl-7-(2,3R-dimethyl-1,2,3,4-tetrahydro-pyrrolo[1,2-a]pyrazin-7-yl)-6-fluoro-8-methoxy-4-oxo-1,4-dihydro-quinoline-3-carboxylic acid; 1-Cyclopropyl-6-fluoro-7-(3R-hydroxymethyl-2-methyl-1,2,3,4-tetrahydro-pyrrolo[1,2-a]pyrazin-7-yl)-8-methoxy-4-oxo-1,4-dihydro-quinoline-3-carboxylic acid; 1-Cyclopropyl-6-fluoro-8-methoxy-7-(1S-methyl-1,2,3,4-tetrahydro-pyrrolo[1,2-a]pyrazin-7-yl)-4-oxo-1,4-dihydro-quinoline-3-carboxylic acid; 1-Cyclopropyl-6-fluoro-8-methoxy-7-(1R-methyl-1,2,3,4-tetrahydro-pyrrolo[1,2-a]pyrazin-7-yl)-4-oxo-1,4-dihydro-quinoline-3-carboxylic acid; 1-Cyclopropyl-7-(1R,3S-dimethyl-1,2,3,4-tetrahydro-pyrrolo[1,2-a]pyrazin-7-yl)-6-fluoro-8-methoxy-4-oxo-1,4-dihydro-quinoine-3-carboxylic acid; 1-Cyclopropyl-6-fluoro-7-(3S-methyl-1,2,3,4-tetrahydro-pyrrolo[1,2-a]pyrazin-7-yl)-4-oxo-1,4-dihydro-quinoline-3-carboxylic acid; 1-Cyclopropyl-7-(2,3S-dimethyl-1,2,3,4-tetrahydro-pyrrolo[1,2-a]pyrazin-7-yl)-6-fluoro-4-oxo-1,4-dihydro-quinoline-3-carboxylic acid; 1-Cyclopropyl-6-fluoro-7-(1S-methyl-1,2,3,4-tetrahydro-pyrrolo[1,2-a]pyrazin-7-yl)-4-oxo-1,4-dihydro-quinoline-3-carboxylic acid; 1-Cyclopropyl-6-fluoro-7-(1R-methyl-1,2,3,4-tetrahydro-pyrrolo[1,2-a]pyrazin-7-yl)-4-oxo-1,4-dihydro-quinoline-3-carboxylic acid; 8-Fluoro-3S-methyl-9-(3S-methyl-1,2,3,4-tetrahydro-pyrrolo[1,2-a]pyrazin-7-yl)-6-oxo-2,3-dihydro-6H-1-oxa-3a-aza-phenalene-5-carboxylic acid; 8-Fluoro-3S-methyl-9-(1S-methyl-1,2,3,4-tetrahydro-pyrrolo[1,2-a]pyrazin-7-yl)-6-oxo-2,3-dihydro-6H-1-oxa-3a-aza-phenalene-5-carboxylic acid; 1-Cyclopropyl-6-fluoro-7-(3S-methyl-1,2,3,4-tetrahydro-pyrrolo[1,2-a]pyrazin-7-yl)-4-oxo-1,4-dihydro-[1,8]naphthyridine-3-carboxylic acid; 1-Cyclopropyl-6-fluoro-7-(1S-methyl-1,2,3,4-tetrahydro-pyrrolo[1,2-a]pyrazin-7-yl)-4-oxo-1,4-dihydro-[1,8]naphthyridine-3-carboxylic acid; 1-(2,4-Difluoro-phenyl)-6-fluoro-7-(3S-methyl-1,2,3,4-tetrahydro-pyrrolo[1,2-a]pyrazin-7-yl)-4-oxo-4-oxo-1,4-dihydro-[1,8]naphthyridine-3-carboxylic acid; 1-(2,4-Difluoro-phenyl)-6-fluoro-7-(1S-methyl-1,2,3,4-tetrahydro-pyrrolo[1,2-a]pyrazin-7-yl)-4-oxo-1,4-dihydro-[1,8]naphthyridine-3-carboxylic acid; 1-Cyclopropyl-8-difluoromethoxy-6-fluoro-7-(1S-methyl-1,2,3,4-tetrahydro-pyrrolo[1,2-a]pyrazin-7-yl)-4-oxo-1,4-dihydro-quinoline-3-carboxylic acid; 7-(2-Benzyl-1,2,3,4-tetrahydro-pyrrolo[1,2-a]pyrazin-7-yl)-1-(2,4-difluoro-phenyl)-6-fluoro-4-oxo-1,4-dihydro-[1,8]naphthyridine-3-carboxylic acid; 7-(2-Benzyl-1,2,3,4-tetrahydro-pyrrolo[1,2-a]pyrazin-7-yl)-1-cyclopropyl-6-fluoro-8-methoxy-4-oxo-1,4-dihydro-quinoline-3-carboxylic acid; 1-Cyclopropyl -6-fluoro-7-(1,2,3,4,6,8a-hexahydro-pyrrolo[1,2-a]pyrazin-7-yl)-8-methoxy-4-oxo-1,4-dihydro-quinoline-3-carboxylic acid; 8-Fluoro-9-(1,2,3,4,6,8a-hexahydro-pyrrolo[1,2-a]pyrazin-7-yl)-3S-methyl-6-oxo-2,3-dihydro-6H-1-oxa-3a-aza-phenalene-5-carboxylic acid; 1-Cyclopropyl-6-fluoro-8-methoxy-4-oxo-7-(1,2,3,4-tetrahydro-pyrrolo[1,2-a]pyrazin-7-yl)-1,4-dihydro-quinoline-3-carboxylic acid; 8-Fluoro-3S-methyl-6-oxo-9-(1,2,3,4-tetrahydro-pyrrolo[1,2-a]pyrazin-7-yl)-2,3-dihydro-6H-1-oxa-3a-aza-phenalene-5-carboxylic acid; and optical isomers, diastereomers, enantiomers, pharmaceutically acceptable salts, hydrates, and prodrugs thereof.

In another embodiment of the present invention, the compound of formula (I) is selected from the group consisting of 1-Cyclopropyl-8-difluoromethoxy-4-oxo-7-(1,2,3,4-tetrahydro-pyrrolo[1,2-a]pyrazin-7-yl)-1,4-dihydro-quinoline-3-carboxylic acid; 1-Cyclopropyl-6-fluoro-8-methoxy-7-(3S-methyl-1,2,3,4-tetrahydro-pyrrolo[1,2-a]pyrazin-7-yl)-4-oxo-1,4-dihydro-quinoline-3-carboxylic acid; 1-Cyclopropyl-6-fluoro-8-methoxy-7-(3R-methyl-1,2,3,4-tetrahydro-pyrrolo[1,2-a]pyrazin-7-yl)-4-oxo-1,4-dihydro-quinoline-3-carboxylic acid; 1-Cyclopropyl-7-(2,3S-dimethyl-1,2,3,4-tetrahydro-pyrrolo[1,2-a]pyrazin-7-yl)-6-fluoro-8-methoxy-4-oxo-1,4-dihydro-quinoline-3-carboxylic acid; 1-Cyclopropyl-6-fluoro-8-methoxy-7-(1S-methyl-1,2,3,4-tetrahydro-pyrrolo[1,2-a]pyrazin-7-yl)-4-oxo-1,4-dihydro-quinoline-3-carboxylic acid; 1-Cyclopropyl-6-fluoro-7-(3S-methyl-1,2,3,4-tetrahydro-pyrrolo[1,2-a]pyrazin-7-yl)-4-oxo-1,4-dihydro-[1,8]naphthyridine-3-carboxylic acid; 1-Cyclopropyl-6-fluoro-8-methoxy-4-oxo-7-(1,2,3,4-tetrahydro-pyrrolo[1,2-a]pyrazin-7-yl)-1,4-dihydro-quinoline-3-carboxylic acid; and optical isomers, diastereomers, enantiomers, pharmaceutically acceptable salts, hydrates, and prodrugs thereof.

Additional embodiments of the present invention, include those wherein the substituents selected for one or more of the variables defined herein (i.e. A, R⁰, R¹, R², R³, R⁴, etc.) are independently selected to be any individual substituent or any subset of substituents selected from the complete list as defined herein. In another embodiment of the present invention is any single compound or subset of compounds selected from the representative compounds listed in Tables 1 and 2 below.

Representative compounds of the present invention are as listed in Tables 1 and 2, below.

TABLE 1
Representative Compounds of Formula (I)

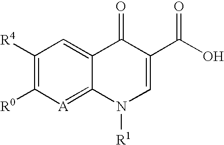

| ID No. | A | R¹ | R⁴ | R⁰ |
|---|---|---|---|---|
| 1 | C—OCHF₂ | cyclopropyl | H | 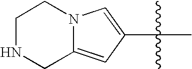 |
| 4 | C—OCHF₂ | cyclopropyl | H | 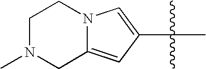 |
| 7 | C—OCHF₂ | cyclopropyl | H | 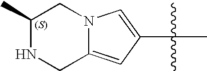 |
| 8 | C—OCHF₂ | cyclopropyl | H | 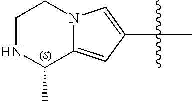 |
| 9 | C—OCHF₂ | cyclopropyl | H | 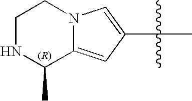 |
| 10 | C—OCHF₂ | cyclopropyl | H | 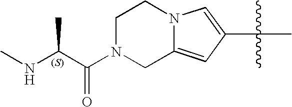 |
| 29 | C—OCHF₂ | cyclopropyl | F | 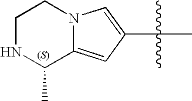 |
| 11 | C—OCH₃ | cyclopropyl | F | 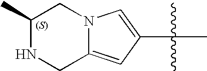 |
| 12 | C—OCH₃ | cyclopropyl | F | 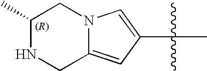 |
| 13 | C—OCH₃ | cyclopropyl | F | 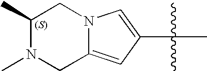 |
| 14 | C—OCH₃ | cyclopropyl | F | 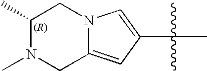 |
| 15 | C—OCH₃ | cyclopropyl | F | 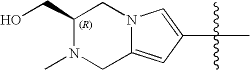 |
| 16 | C—OCH₃ | cyclopropyl | F | 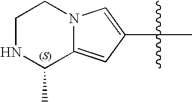 |
| 17 | C—OCH₃ | cyclopropyl | F | 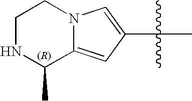 |
| 18 | O—OCH₃ | cyclopropyl | F | 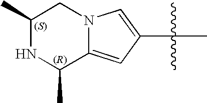 |
| 31 | C—OCH₃ | cyclopropyl | F | 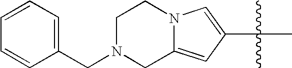 |
| 34 | C—OCH₃ | cyclopropyl | F | 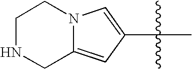 |
| 19 | CH | cyclopropyl | F | 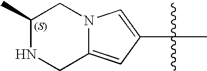 |
| 20 | CH | cyclopropyl | F | 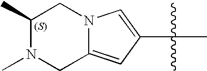 |
| 21 | CH | cyclopropyl | F | 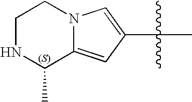 |
| 22 | CH | cyclopropyl | F | 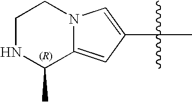 |
| 25 | N | cyclopropyl | F | 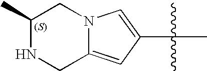 |
| 26 | N | cyclopropyl | F | 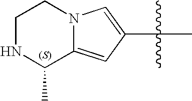 |
| 27 | N | 2,4-difluorophenyl | F | 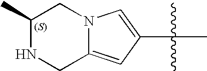 |
| 28 | N | 2,4-difluorophenyl | F | 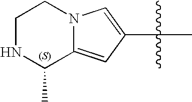 |
| 30 | N | 2,4-difluorophenyl | F | 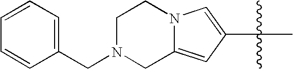 |
| 2 | C—OCHF₂ | cyclopropyl | H | 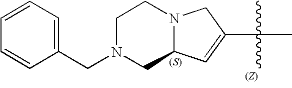 |
| 32 | C—OCH₃ | cyclopropyl | F | 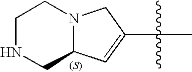 |
| 3 | C—OCHF₂ | cyclopropyl | H | 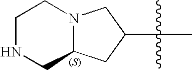 |
| 5 | C—OCHF₂ | cyclopropyl | H | 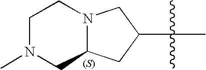 |
| 6 | C—OCHF₂ | cyclopropyl | H | 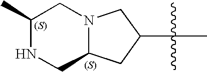 |

TABLE 2
Representative Compounds of Formula (Id)

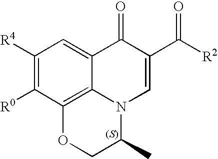

| ID No. | R² | R⁴ | R⁰ |
|---|---|---|---|
| 23 | OH | F | 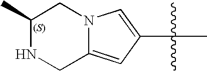 |
| 24 | OH | F | 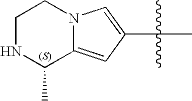 |
| 35 | OH | F | 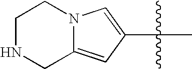 |
| 33 | OH | F | 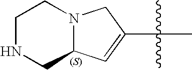 |

In an embodiment, the present invention is directed to compounds of formula (I) whose MIC (minimal inhibitory concentration) against strain A as measured according to the procedure described in Example 36 is less than or equal to about 0.25 μg/mL, preferably less than or equal to about 0.12 μg/mL, more preferably less than or equal to about 0.06 μg/mL. In an embodiment, the present invention is directed to compounds of formula (I) whose MIC (minimal inhibitory concentration) against strain B as measured according to the procedure described in Example 36 is less than or equal to about 0.06 μg/mL, preferably less than or equal to about 8 μg/mL, more preferably less than or equal to about 4 μg/mL. In an embodiment, the present invention is directed to compounds of formula (I) whose MIC (minimal inhibitory concentration) against strain C as measured according to the procedure described in Example 36 is less than or equal to about 16 μg/mL, preferably less than or equal to about 8 μg/mL, more preferably less than or equal to about 4 μg/mL. In an embodiment, the present invention is directed to compounds of formula (I) whose MIC (minimal inhibitory concentration) against strain D as measured according to the procedure described in Example 36 is less than or equal to about 16 μg/mL, preferably less than or equal to about 8 μg/mL, more preferably less than or equal to about 4 μg/mL. In an embodiment, the present invention is directed to compounds of formula (I) whose MIC (minimal inhibitory concentration) against strain E as measured according to the procedure described in Example 36 is less than or equal to about 4 μg/mL, preferably less than or equal to about 2 μg/mL, more preferably less than or equal to about 1 μg/mL.

As used herein, the terms “halo” or “halogen” shall mean fluoro, chloro, bromo or iodo.

As used herein, the prefix “Cₓ-C_{y}” wherein x and y are numbers shall denote the number of carbon atoms present in a particular functional group. For example, the term “C₁-C₄alkyl” denotes any straight or branched chain alkyl as herein define of between 1 and 4 carbon atoms, inclusive. Similarly, the term “C₂-C₄alkenyl” shall denote an alkenyl group of between 2 and 4 carbon atoms inclusive.

As used herein, the term “alkyl” shall mean a saturated, straight or branched hydrocarbon chain having 1 to 15 carbons. For example, alkyl radicals include methyl, ethyl, propyl, isopropyl, butyl, isobutyl, sec-butyl, t-butyl, pentyl and the like. The term “halogenated alkyl” shall mean any alkyl group as defined above substituted with one to five halogen atoms, preferably with at least one halogen atom, preferably substituted with a least one fluoro atom. Suitable examples include but are not limited to —CF₃, —CH₂—CF₃, —CF₂—CF₂—CF₂—CF₃, and the like. Similarly, the term “fluorinated alkyl” shall mean any alkyl group as defined above substituted with one to five fluoro atoms, preferably with at least one fluoro atom. Suitable examples include but are not limited to —CF₃, —CH₂—CF₃, —CF₂—CF₂—CF₂—CF₃, and the like. Similarly, the term “hydroxy substituted C₁₋₄alkyl” shall mean any C₁₋₄alkyl as defined herein substituted with one or more, preferably one hydroxy group. Preferably, the C₁₋₄alkyl is substituted with one hydroxy group at the terminal carbon, for example —CH₂—OH, —CH₂CH₂—OH, and the like.

The term “alkenyl” shall mean a straight or branched hydrocarbon chain having at least one carbon-carbon double bond and having 2 to 15 carbon atoms. Similarly, the term “alkynyl” shall mean a straight or branched hydrocarbon chain having at least one carbon-carbon triple bond and having 2 to 15 carbon atoms.

The term “alkoxy” shall denote an oxygen ether radical of the above described straight or branched chain alkyl groups (i.e. a group of the formula —O-alkyl). For example, methoxy, ethoxy, n-propoxy, sec-butoxy, t-butoxy, n-hexyloxy and the like. The term “halogenated alkoxy” shall mean any alkoxy group as defined above substituted with one to five halogen atoms, preferably with at least one halogen atom, preferably substituted with a least one fluoro atom. Suitable examples include but are not limited to —OCF₃, —OCHF₂, —OCH₂—CF₃, —OCF₂—CF₂—CF₂—CF₃, and the like. Similarly, the term “fluorinated alkoxy” shall mean any alkoxy group as defined above substituted with one to five fluoro atoms, preferably with at least one halogen atom, preferably substituted with a least one fluoro atom. Suitable examples include but are not limited to —OCF₃, —OCHF₂, —OCH₂—CF₃, —OCF₂—CF₂—CF₂—CF₃, and the like.

The term “alkylthio” shall mean —S-alkyl. Suitable examples include but are not limited to methylthio, (—S—CH₃), ethyl-thio, isopropyl-thio, and the like.

The term “C₃-C₈cycloalkyl” shall mean any stable 3 to 8 membered (preferably 5 to 6 membered) monocyclic, saturated ring system. Suitable examples include cyclopropyl, cyclobutyl, cyclopentyl, cyclohexyl, cycloheptyl and cyclooctyl. Further, wherein the cycloalkyl is optionally substituted, said substitution may be at any of the carbon atoms of the cycloalkyl group.

The term “aryl” shall refer to unsubstituted carbocylic aromatic groups such as phenyl, naphthyl, and the like. The term “aralkyl” shall mean an -(alkyl)-(aryl), such as benzyl, phenethyl, and the like; preferably the aralkyl group is of the formula —(C₁₋₄alkyl)-(aryl).

The term “heteroaryl” shall denote any five or six membered monocyclic aromatic ring structure containing at least one heteroatom selected from the group consisting of O, N and S, optionally containing one to three additional heteroatoms independently selected from the group consisting of O, N and S; or a nine or ten membered bicyclic aromatic ring structure containing at least one heteroatom selected from the group consisting of O, N and S, optionally containing one to four additional heteroatoms independently selected from the group consisting of O, N and S. The heteroaryl group may be attached at any heteroatom or carbon atom of the ring such that the result is a stable structure. Unless otherwise noted, the heteroaryl group may be optionally substituted with one or more substituents as herein defined. Examples of suitable heteroaryl groups include, but are not limited to, pyrrolyl, furyl, thienyl, oxazolyl, imidazolyl, pyrazolyl, isoxazolyl, isothiazolyl, triazolyl, thiadiazolyl, pyridyl, pyridazinyl, pyrimidinyl, pyrazinyl, pyranyl, furazanyl, indolizinyl, indolyl, isoindolinyl, indazolyl, benzofuryl, benzothienyl, benzimidazolyl, benzothiazolyl, purinyl, quinolinyl, isoquinolinyl, isothiazolyl, cinnolinyl, phthalazinyl, quinazolinyl, quinoxalinyl, naphthyridinyl, pteridinyl, and the like. Preferred heteroaryl groups include, but are not limited to pyridyl, quinolinyl and isoquinolinyl.

The term “heterocycloalkyl” shall denote any five to seven membered monocyclic, saturated or partially unsaturated ring structure containing at least one heteroatom selected from the group consisting of O, N and S, optionally containing one to three additional heteroatoms independently selected from the group consisting of O, N and S; or a nine to ten membered saturated, partially unsaturated or partially aromatic bicyclic ring system containing at least one heteroatom selected from the group consisting of O, N and S, optionally containing one to four additional heteroatoms independently selected from the group consisting of O, N and S. The heterocycloalkyl group may be attached at any heteroatom or carbon atom of the ring such that the result is a stable structure. Unless otherwise noted, the heterocycloalkyl group may be optionally substituted with one or more substituents as herein defined. Examples of substituted and unsubstituted heterocycloalkyl groups include, but are not limited to, pyrrolinyl, pyrrolidinyl, dioxolanyl, imidazolinyl, imidazolidinyl, pyrazolinyl, pyrazolidinyl, piperidinyl, dioxanyl, morpholinyl, dithianyl, thiomorpholinyl, piperazinyl, trithianyl, indolinyl, chromenyl, 3,4-methylenedioxyphenyl, 2,3-dihydrobenzofuryl, phthalimido (also known as isoindole-1,3-dione), isoindolinyl, and the like. Preferred heterocycloalkyl groups include, but are not limited to phthalimido.

As used herein, unless otherwise noted, the term “5 to 6 membered nitrogen containing saturated ring structure” shall mean a ring structure consisting of 5 to 6 ring atoms, where at least one of the ring atoms is nitrogen, and wherein one to two additional ring atoms are selected from the group consisting of N, O and S. Suitably examples include, but are not limited to pyrrolidinyl, piperidinyl, morpholinyl, thiomorpholinyl, piperazinyl, and the like. Preferred 5 to 6 membered nitrogen containing saturated ring structures include, pyrrolidinyl, piperidinyl, piperazinyl and morpholinyl. In an embodiment of the present invention, the 5 to 6 membered nitrogen containing saturated ring structure is a 6 membered nitrogen containing saturated ring structure. In another embodiment of the present invention, the 5 to 6 membered nitrogen containing saturated ring structure is selected from the group consisting of pyrrolidinyl, piperidinyl, piperazinyl and morpholinyl.

The term “aryloxy” shall mean a group of the formula —O-aryl, wherein the aryl group is as defined above. Similarly, the term “heteroaryloxy” shall mean a group of the formula —O-heteroaryl, wherein the heteroaryl group is as defined above. Similarly, the term “heterocycloalkyloxy” shall mean a group of the formula —O-heterocycloalkyl, wherein the heterocycloalkyl group is as defined above.

The term “acyl” shall mean an organic radical having 2 to 6 carbon atoms (straight chain or branched) derived from an organic acid by removal of the hydroxyl. Suitable examples include but are not limited to acetyl, propionyl and the like. The term “acyloxy” shall mean a group of the formula —O-acyl, wherein the acyl group is as defined above. Suitable examples include but are not limited to acetoxy, propionoxy, and the like.

Unless specified otherwise, it is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein. One skilled in the art will further recognize that substituents may be bound to any of the atoms of a particular group (including, but not limited to C, N or S atoms), provided that the substitution results in a stable structure and does not violate valence rules.

Some of the compounds of the present invention may have trans and cis isomers. In addition, where the processes for the preparation of the compounds according to the invention give rise to mixture of stereoisomers, these isomers may be separated by conventional techniques such as preparative chromatography. The compounds may be prepared as a single stereoisomer or in racemic form as a mixture of some possible stereoisomers. The non-racemic forms may be obtained by either synthesis or resolution. The compounds may, for example, be resolved into their component enantiomers by standard techniques, such as the formation of diastereomeric pairs by salt formation. The compounds may also be resolved by covalent linkage to a chiral auxiliary, followed by chromatographic separation and/or crystallographic separation, and removal of the chiral auxiliary. Alternatively, the compounds may be resolved using chiral chromatography.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Preferably, wherein the compound is present as an enantiomer, the enantiomer is present at an enantiomeric excess of greater than or equal to about 80%, more preferably, at an enantiomeric excess of greater than or equal to about 90%, more preferably still, at an enantiomeric excess of greater than or equal to about 95%, more preferably still, at an enantiomeric excess of greater than or equal to about 98%, most preferably, at an enantiomeric excess of greater than or equal to about 99%. Similarly, wherein the compound is present as a diastereomer, the diastereomer is present at a diastereomeric excess of greater than or equal to about 80%, more preferably, at a diastereomeric excess of greater than or equal to about 90%, more preferably still, at a diastereomeric excess of greater than or equal to about 95%, more preferably still, at a diastereomeric excess of greater than or equal to about 98%, most preferably, at a diastereomeric excess of greater than or equal to about 99%.

Furthermore, some of the crystalline forms for the compounds of the present invention may exist as polymorphs and as such are intended to be included in the present invention. In addition, some of the compounds of the present invention may form solvates with water (i.e., hydrates) or common organic solvents, and such solvates are also intended to be encompassed within the scope of this invention.

Under standard nomenclature used throughout this disclosure, the terminal portion of the designated side chain is described first, followed by the adjacent functionality toward the point of attachment. Thus, for example, a “phenylC₁-C₆alkyl-aminocarbonyl-C₁-C₆alkyl” substituent refers to a group of the formula

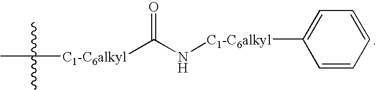

One skilled in the art will recognize that in the ring structures of R⁰ the “nitrogen at the ring fusion” is the nitrogen as indicated by the arrow in the below representations:

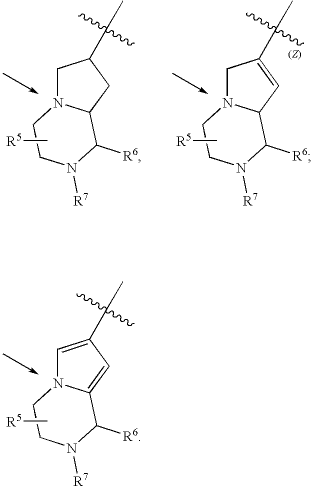

One skilled in the art will further recognize that wherein the compounds of formula (I) A is CR²⁰ and R¹ and R²⁰ are taken together to form

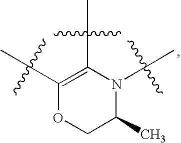

(when unbound to the rest of the compound of formula (I), known as 3S-methyl-3,4-dihydro-2H-[1,4]oxazine, and which is fused to the compound of formula (I) through the 4-, 5- and 6-positions of the oxazine) then the corresponding compounds, hereinafter collectively referred to as compounds of formula (Id), are of the following structure:

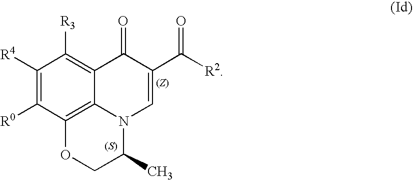

Abbreviations used in the specification, particularly the Schemes and Examples, are as follows:

BOC or Boc=t-Butoxycarbonyl (i.e. —C(O)O—C(CH₃)₃)

BOC₂O=Boc-anhydride

Cbz=Benzyloxycarbonyl

DBU=1,8-Diazabicyclo[5.4.0]undec-7-ene

DCC=dicyclohexylcarbodiimide

DCE=Dichloroethane

DCM=Dichloromethane

DDQ=2,3-Dichloro-5,6-dicyanobenzoquinone

DIBAL-H Diisobutyl Aluminum Hydride

DIPEA Diisopropylethylamine

DME=1,2-Dimethoxyethane

DMAP=4-(Dimethylamino)pyridine

DMF=N,N-Dimethylformamide

DMSO=Dimethylsulfoxide

EDCI=1-Ethyl-3-(3-dimethylaminopropyl)carbodiimide

EtOAc=Ethyl Acetate

EtOH=Ethanol

HPLC=High Performance Liquid Chromatography

LAH=Lithium Aluminum Hydride

MeOH=Methanol

MIC=Minimal Inhibitory Concentration

PD/C=Palladium on Carbon Catalyst

Pd₂(dba)₃=Tris(dibenzylideneacetone)dipalladium (0)

Pd(PPh₃)₄=Tetrakis(triphenylphosphine)palladium (0)

Pd(PPh₃)₂Cl₂=Bis(triphenylphosphine)palladium (11) Chloride

PPh₃=Triphenylphosphine

TBAF=Tetrabutylammonium Fluoride

TBS=t-Butyldimethylsilyl

TMSCI=t-Butyldimethylsilyl Chloride

TEA or Et₃N=Triethylamine

TFA=Trifluoromethylsulfonyl- (i.e. —SO₂—CF₃)

TFA=Trifluoroacetic Acid

THF=Tetrahydrofuran

The term “subject” as used herein, refers to an animal, preferably a mammal, most preferably a human, who has been the object of treatment, observation or experiment. Preferably, the subject has experienced and/or exhibited at least one symptom of the disease or disorder to be treated and/or prevented.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

The term “prophylactically effective amount” as used herein, means that amount of active compound or pharmaceutical agent that prevents the development of a condition, symptoms or manifestations thereof associated with bacterial infection. Thus it elicits the biological or medicinal response in a tissue system, animal or human that is being sought by a researcher, veterinarian, medical doctor or other clinician, which includes alleviation of the symptoms of the disease or disorder being treated.

The term “drug-resistant” or “drug-resistance” refers to the characteristics of a microbe to survive in the presence of a currently available antimicrobial agent such as an antibiotic at its routine, effective concentration.

As used herein, the term “composition” is intended to encompass a product comprising the specified ingredients in the specified amounts, as well as any product which results, directly or indirectly, from combinations of the specified ingredients in the specified amounts.

One skilled in the art will recognize that, where not otherwise specified, the reaction step(s) is performed under suitable conditions, according to known methods, to provide the desired product.

One skilled in the art will recognize that, in the specification and claims as presented herein, wherein a reagent or reagent class/type (e.g. base, solvent, etc.) is recited in more than one step of a process, the individual reagents are independently selected for each reaction step and may be the same or different from each other. For example wherein two steps of a process recite an organic or inorganic base as a reagent, the organic or inorganic base selected for the first step may be the same or different than the organic or inorganic base of the second step.

One skilled in the art will further recognize that wherein a reaction step of the present invention may be carried out in a variety of solvents or solvent systems, said reaction step may also be carried out in a mixture of the suitable solvents or solvent systems.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any range therein.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

As used herein, unless otherwise noted, the term “nitrogen protecting group” shall mean a group which may be attached to a nitrogen atom to protect said nitrogen atom from participating in a reaction and which may be readily removed following the reaction. Suitable nitrogen protecting groups include, but are not limited to carbamates—groups of the formula —C(O)O—R wherein R is for example methyl, ethyl, t-butyl, benzyl, phenethyl, CH₂═CH—CH₂—, and the like; amides—groups of the formula —C(O)—R′ wherein R′ is for example methyl, phenyl, trifluoromethyl, and the like; N-sulfonyl derivatives—groups of the formula —SO₂—R″ wherein R″ is for example tolyl, phenyl, trifluoromethyl, 2,2,5,7,8-pentamethylchroman-6-yl-, 2,3,6-trimethyl-4-methoxybenzene, and the like. Other suitable nitrogen protecting groups may be found in texts such as T. W. Greene & P. G. M. Wuts, Protective Groups in Organic Synthesis, John Wiley & Sons, 1991.

As used herein, unless otherwise noted, the term “oxygen protecting group” shall mean a group which may be attached to a oxygen atom to protect said oxygen atom from participating in a reaction and which may be readily removed following the reaction. Suitable oxygen protecting groups include, but are not limited to, acetyl, benzoyl, t-butyl-dimethyl silyl, trimethylsilyl (TMS), MOM, THP, and the like. Other suitable oxygen protecting groups may be found in texts such as T. W. Greene & P. G. M. Wuts, Protective Groups in Organic Synthesis, John Wiley & Sons, 1991.

As used herein, unless otherwise noted, the term “leaving group” shall mean a charged or uncharged atom or group that departs during a substitution or displacement reaction. Suitable examples include, but are not limited to, Br, Cl, I, triflate, tosylate, and the like.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts.

Thus, representative pharmaceutically acceptable salts include, but are not limited to, the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laureate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids that may be used in the preparation of pharmaceutically acceptable salts include, but are not limited to, the following: acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid and undecylenic acid.

Representative bases that may be used in the preparation of pharmaceutically acceptable salts include, but are not limited to, the following: ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, sodium hydroxide, triethanolamine, tromethamine and zinc hydroxide.

The present invention includes within its scope prodrugs of the compounds of this invention. In general, such prodrugs will be functional derivatives of the compounds that are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

### General Reaction Scheme for Compound Preparation

In making the compounds of the invention, the order of synthetic steps may be varied to increase the yield of desired product. In addition, the skilled artisan will also recognize the judicious choice of reactions, solvents, and temperatures are an important component in successful synthesis. While the determination of optimal conditions, etc. is routine, it will be understood that a variety of compounds can be generated in a similar fashion, using the guidance of the schemes below.

It is recognized that the skilled artisan in the art of organic chemistry can readily carry out standard manipulations of the organic compounds without further direction; that is, it is well within the scope and practice of the skilled artisan to carry out such manipulations. These include, but are not limited to, reductions of carbonyl compounds to their corresponding alcohols, oxidations, acylations, aromatic substitutions, both electrophilic and nucleophilic, etherifications, esterification and saponification and the like. Examples of these manipulations are discussed in standard texts such as March, Advanced Organic Chemistry (Wiley), Carey and Sundberg, Advanced Organic Chemistry (Vol. 2), Feiser & Feiser, Reagents for Organic Synthesis (16 volumes), L. Paquette, Encyclopedia of Reagents for Organic Synthesis (8 volumes), Frost & Fleming, Comprehensive Organic Synthesis (9 volumes) and the like.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. Examples of these manipulations can be found for example in T. Greene, Protecting Groups in Organic Synthesis.

The starting materials used in preparing the compounds of the invention are known, made by published synthetic methods or available from commercial vendors.

The compounds of formula (I) wherein R² is selected from the group consisting of C₁₋₄alkoxy and benzyloxy and wherein R⁷ is other than hydrogen, may be prepared according to the general process as outlined in Scheme 1, below.

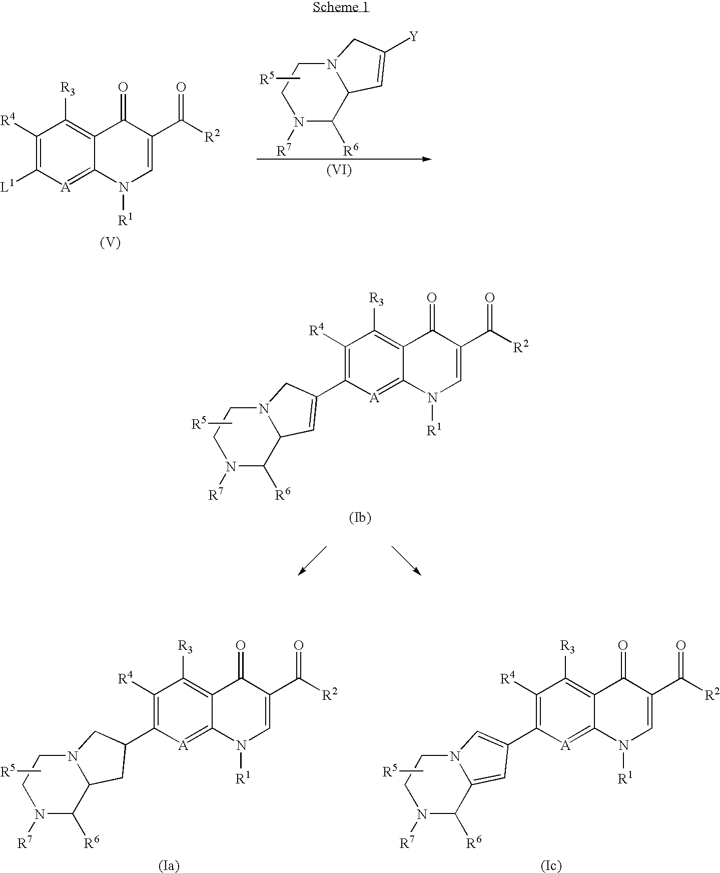

Accordingly, a suitably substituted compound of formula (V), wherein R² is selected from the group consisting of C₁₋₄alkoxy and benzyloxy; and wherein L¹ is a suitably selected leaving group such as Br, I, Cl, —O—SO₂CF₃ (i.e. triflate), and the like, preferably Br, Cl or triflate, a known compound or compound prepared by known methods, is reacted with a suitably substituted compound of formula (VI), wherein Y is suitably selected boronic acid (i.e. —B(OH)₂), boronic ester (such as

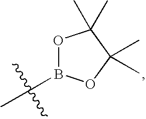

and the like), or trialkyltin (such as trimethyltin, and the like), a known compound or compound prepared by known methods; according to known methods; to yield the corresponding compound of formula (Ib).

For example, wherein the compound of formula (V), L¹ is a leaving group such as Br, Cl, —O—SO₂—CF₃, and the like; and wherein the compound of formula (VI), Y is

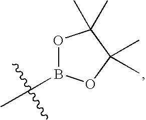

the compound of formula (V) is reacted with the compound of formula (VI); in the presence of a suitably selected catalyst, such as Pd(PPh₃)₄, Pd₂(dba)₃, and the like; in the presence of a suitably selected inorganic base such as CsF, Cs₂CO₃, K₂CO₃, and the like, preferably CsF; in an organic solvent, such as THF, 1,4-dioxane, DMF, and the like; at a temperature in the range of from about 40° C. to about 100° C., preferably at a temperature of about 65° C.; preferably for a time period in the range of from about 0.5 to 48 hours; to yield the corresponding compound of formula (Ib).

The compound of formula (Ib) is reacted with a suitably selected reducing agent such as hydrogen gas, and the like; in the presence of a catalyst such as Pd/C, and the like; in an organic solvent such as methanol, ethanol, ethyl acetate, and the like; preferably at about room temperature; preferably for a time period in the range of from about 1 to 24 hours; to yield the corresponding compound of formula (Ia), wherein R² is selected from the group consisting of C₁₋₄alkoxy and benzyloxy. Preferably, the compound of formula (Ib) is reduced with H₂ gas in the presence of a catalyst when R² is C₁₋₄alkoxy.

Alternatively, the compound of formula (Ib) is oxidized to the corresponding compound of formula (Ic). In an example, the compound of formula (Ib) is reacted with a suitably selected oxidizing agent such as air in the presence of a suitably selected catalyst such as Pd/C, and the like; in an organic solvent such as methanol, ethanol, and the like; preferably at a temperature in the range of from about room temperature to about 70° C.; preferably for a time period in the range of from about 1 to about 72 hours; to yield the corresponding compound of formula (Ic), wherein R² is selected from the group consisting of C₁₋₄alkoxy and benzyloxy. In another example, the compound of formula (Ib) is reacted with a suitably selected oxidizing agent such as DDQ, and the like; in an organic solvent such as DCM, DCE, and the like; preferably at about room temperature; preferably for a time period in the range of from about 1 hour to about 72 hours; to yield the corresponding compound of formula (Ic), wherein R² is selected from the group consisting of C₁₋₄alkoxy and benzyloxy.

Compounds of formula (I) wherein R² is hydroxy, may be prepared from the corresponding compound of formula (I) wherein R² is selected from the group consisting of C₁₋₄alkoxy and benzyloxy, by de-protecting according to known methods. For example, the compound of formula (I) wherein R² is selected from the group consisting of C₁₋₄alkoxy and benzyloxy may be de-protected by reacting with a suitably selected base such as NaOH, KOH, and the like; in an aqueous solvent mixture, such as THF/MeOH/H₂O, THF/H₂O, MeOH/H₂O, and the like.

Compounds of formula (I) wherein R⁷ is hydrogen may be similarly prepared according to the process as outlined in Scheme 1 above, by substituting a suitable substituted compound of formula (VII)

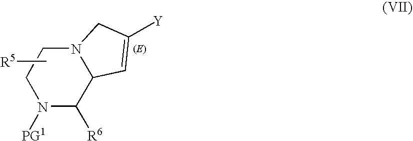

wherein PG¹ is a suitably selected nitrogen protecting group such as Boc, CBz, and the like, preferably Boc; for the compound of formula (VI); to yield the corresponding compound of formula (Id).

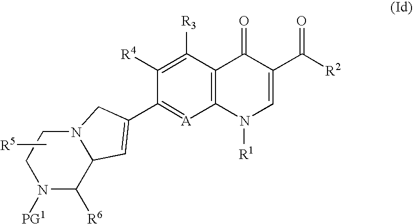

The compound of formula (Id) is further optionally reduced as described above, to yield the corresponding compound of formula (Ie)

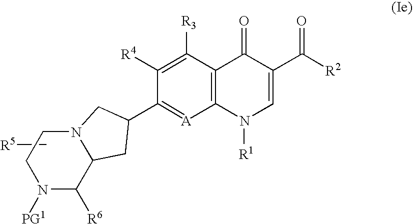

or the compound of formula (Id) is further optionally oxidized as described above, to yield the corresponding compound of formula (If),

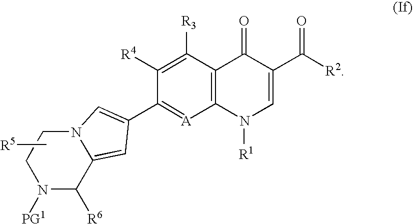

The compound of formula (Id), the compound of formula (Ie) or the compound of formula (If) is then de-protected according to known methods. For example, wherein PG¹ is Boc, the compound is de-protected by reacting with a suitably selected acid such as TFA, HCl, H₂SO₄, and the like; according to known methods; to yield the corresponding compound of formula (I), wherein R⁷ is hydrogen.

Compounds of formula (I) wherein R⁰ is

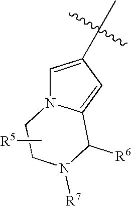

and R⁷ is selected from the group consisting of —C(O)—R⁸ and —C(O)—(C₁₋₄alkyl)-NR⁹R¹⁰ may alternatively be prepared according to the process outlined in Scheme 2 below.

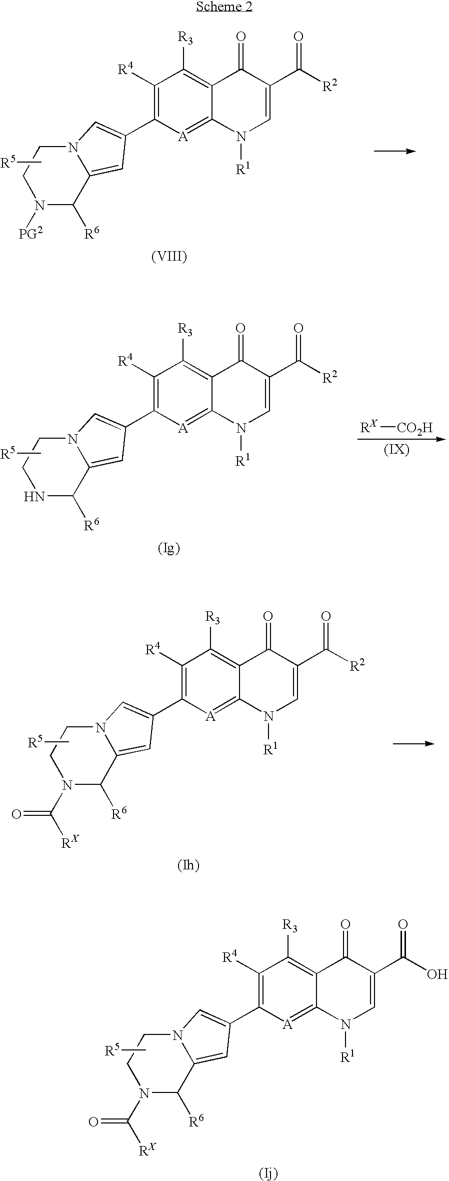

Accordingly, a suitably substituted compound of formula (VIII), wherein R² is selected from the group consisting of C₁₋₄alkoxy and benzyloxy; and wherein PG² is a suitably selected nitrogen protecting group such as Boc, CBz, and the like, preferably Boc; is de-protected according to known methods (for example, by reacting with a suitably selected acid such as TFA, HCl, and the like; in an organic solvent such as DCM, DCE, and the like; preferably at a temperature in the range of from about 0° C. to about 37° C.; preferably for a period of time in the range of from about 1 to about 24 hours), to yield the corresponding compound of formula (Ig).

The compound of formula (Ig) is reacted with a suitably substituted compound of formula (IX), wherein R^{X} is selected from the group consisting of —R⁸, —(C₁₋₄alkyl)-NR⁹R¹⁰, wherein R⁹ and R¹⁰ are each other than hydrogen, and —(C₁₋₄alkyl)-NR⁹(PG^{X}), wherein PG^{X} is a suitably selected nitrogen protecting group, preferably Boc or CBz, a known compound or compound prepared by known methods; in the presence of a suitably selected coupling agent or coupling agent system, such as dicyclohexylcarbodiimide (DCC), 1-ethyl-3-(3-dimethylaminopropyl)carbodiimide (EDCI), and the like; an optional in the presence of a suitably selected acylation catalyst, such as 4-(dimethylamino)pyridine (DMAP), and the like; in an organic solvent such as methylene chloride, DMF, and the like; preferably at a temperature in the range of from about 0° C. to about 37° C.; preferably for a period of time in the range of from about 1 hour to about 48 hours; to yield the corresponding compound of formula (Ih).

The compound of formula (Ih) is further optionally reacted with a suitably selected base such as NaOH, KOH, and the like; in an aqueous solvent mixture, such as THF/MeOH/H₂O, THF/H₂O, MeOH/H₂O, and the like; preferably at a temperature in the range of from about 0° C. to about 70° C.; preferably for a period of time in the range of from about 5 min to about 12 hours; to yield the corresponding compound of formula (Ij).

Wherein the compound of formula (Ij), R^{X} is —(C₁₋₄alkyl)-NR⁹(PG^{X}); the compound of formula (Ij) may be further de-protected according to known methods to yield the corresponding compound where R^{X} is —(C₁₋₄alkyl)-NHR⁹; and then further, optionally reacted according to known methods, to yield the corresponding compound wherein R^{X} is —(C₁₋₄alkyl)-NR⁹R¹⁰.

One skilled in the art will recognize that compounds of formula (I) wherein R⁰ is

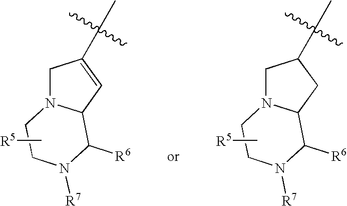

and wherein R⁷ is selected from the group consisting of —C(O)—R⁸ and —C(O)—(C₁₋₄alkyl)—NR⁹R¹⁰ may similarly be prepared according to the process as outlined in Scheme 2 above, by substituting a suitably substituted compound of formula (X)

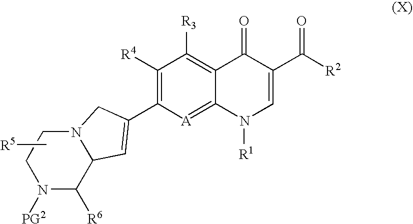

wherein R² is selected from the group consisting of C₁₋₄alkoxy and benzyloxy; or a suitably substituted compound of formula (XI)

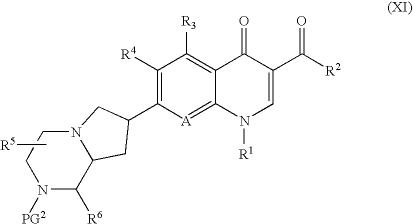

wherein R² is selected from the group consisting of C₁₋₄alkoxy and benzyloxy; respectively, for the compound of formula (VIII).

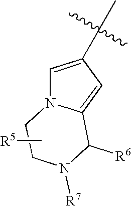

Compounds of formula (I) wherein R⁰ is and R⁷ is selected from the group consisting of C₁₋₄alkyl, C₃₋₈cycloalkyl, —(C₁₋₄alkyl)-C₃₋₈cycloalkyl, aralkyl, —(C₁₋₄alkyl)-heteroaryl, heterocycloalkyl and —(C₁₋₄alkyl)-heterocycloalkyl, may alternatively be prepared according to the process outlined in Scheme 3 below.

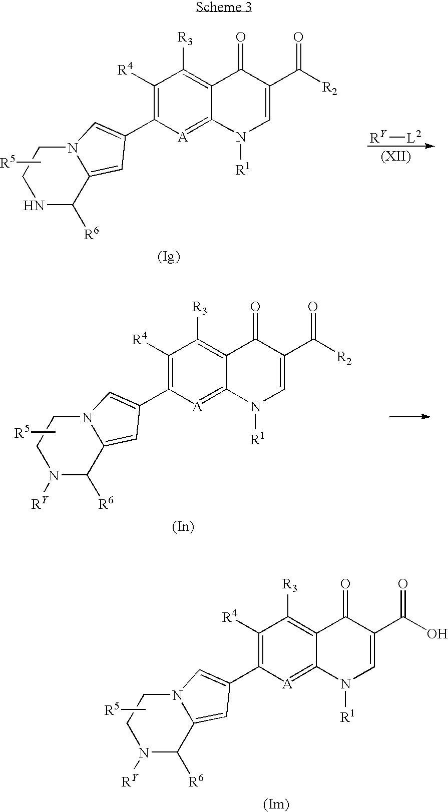

Accordingly, a suitably substituted compound of formula (Ig), wherein R² is selected from the group consisting of C₁₋₄alkoxy and benzyloxy is reacted with a suitably substituted compound of formula (XII), wherein R^{Y} is selected from the group consisting of C₁₋₄alkyl, C₃₋₈cycloalkyl, —(C₁₋₄alkyl)-C₃₋₈cycloalkyl, aralkyl, —(C₁₋₄alkyl)-heteroaryl, heterocycloalkyl and —(C₁₋₄alkyl)-heterocycloalkyl, and wherein L² is a suitably selected leaving group such as Br, I, tosylate, and the like; in the presence of a base such as TEA, DIPEA, DBU, K₂CO₃, Na₂CO₃, NaHCO₃, and the like; in an organic solvent such as THF, DMF, DCM, and the like; preferably at a temperature in the range of from about 0° C. to about 80° C.; preferably for a period of time in the range of from about 0.5 hours to about 24 hours; to yield the corresponding compound of formula (In).

The compound of formula (In) is further, optionally reacted to yield the corresponding compound of formula (Im). In an example, the compound of formula (In) is reacted with a suitably selected base such as NaOH, KOH, and the like; in an aqueous solvent mixture, such as THF/MeOH/H₂O, THF/H₂O, MeOH/H₂O, and the like; preferably at a temperature in the range of from about 0° C. to about 70° C.; preferably for a period of time in the range of from about 5 min to about 12 hours; to yield the corresponding compound of formula (Im). In another example, the compound of formula (In) is reacted with a suitably selected acid such as HCl, H₂SO₄, and the like; in H₂O; preferably at a temperature in the range of from about 50° C. to about 100° C.; preferably for a period of time in the range of from about 1 hour to about 12 hours; to yield the corresponding compound of formula (Im).

One skilled in the art will recognize that compounds of formula (I) wherein R₀ is

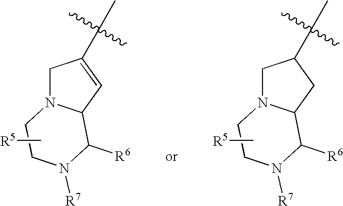

and wherein R⁷ is selected from the group consisting of C₁₋₄alkyl, C₃₋₈cycloalkyl, —(C₁₋₄alkyl)-C₃₋₈cycloalkyl, aralkyl, —(C₁₋₄alkyl)-heteroaryl, heterocycloalkyl and —(C₁₋₄alkyl)-heterocycloalkyl, may similarly be prepared according to the process as outlined in Scheme 3 above, by substituting a suitably substituted compound of formula (Ip)

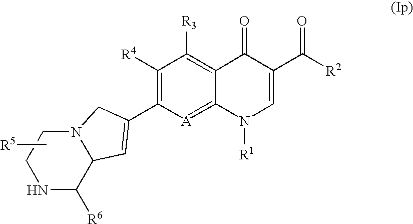

wherein R² is selected from the group consisting of C₁₋₄alkoxy and benzyloxy; or a suitably substituted compound of formula (Iq)

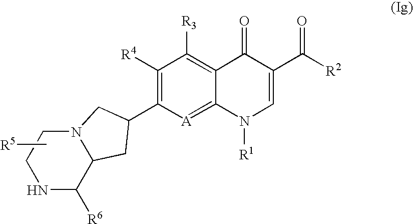

wherein R² is selected from the group consisting of C₁₋₄alkoxy and benzyloxy; respectively, for the compound of formula (Ig).

Compounds of formula (VII) may be prepared according to, for example, the processes outlined in Schemes 4-6, below. Compounds of formula (VII) wherein Y is a boronic ester such as

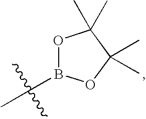

R⁶ is hydrogen, and R⁵ is bound at the alpha position to the N—R⁷ portion of the R⁰ substituent group may be prepared according to the process outlined in Scheme 4 below.

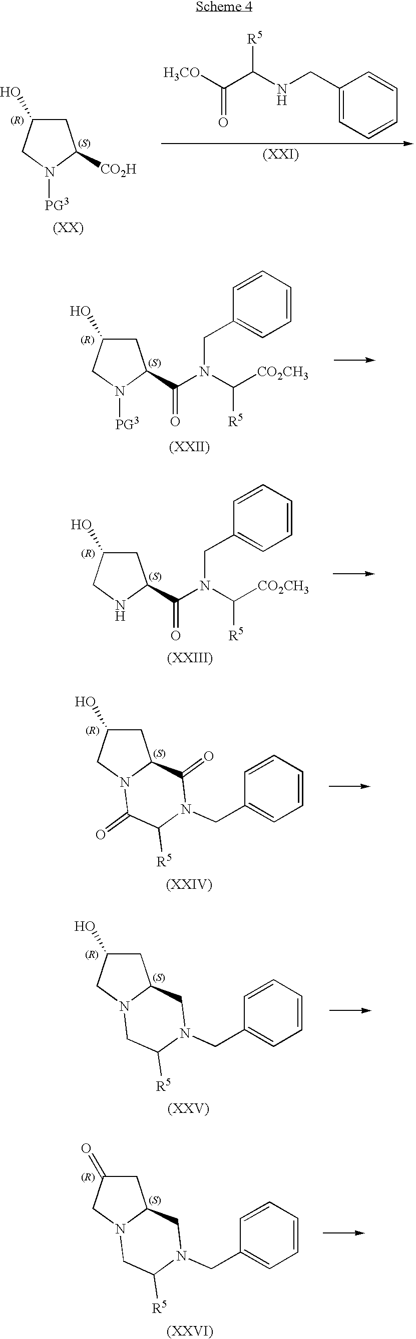

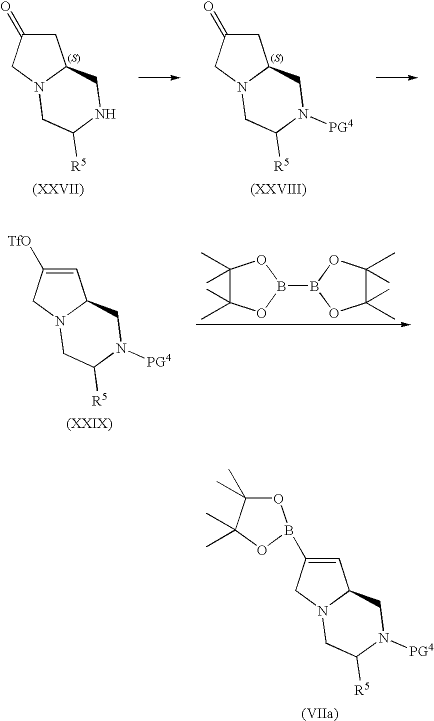

Accordingly, a suitably substituted compound of formula (XX) wherein PG³ is a suitably selected nitrogen protecting group such as Boc, Cbz, and the like, preferably Boc (i.e. N-Boc-4-hydroxy-L-proline), a known compound or compound prepared by known methods, is reacted with a suitably substituted compound of formula (XXI), a known compound or compound prepared by known methods; in the presence of a suitably selected coupling agent, such as dicyclohexylcarbodiimide (DCC), 1-ethyl-3-(3-dimethylaminopropyl)carbodiimide (EDCI), and the like; in an organic solvent such as methylene chloride, chloroform, DMF, and the like; preferably at a temperature in the range of from about 0° C. to about 40° C.; preferably for a period of time in the range of from about 1 hour to about 48 hours; to yield the corresponding compound of formula (XXII).

The compound of formula (XXII) is de-protected according to known methods, to yield the corresponding compound of formula (XXIII). For example, wherein PG³ is Boc, the compound of formula (XXII) is de-protected by reacting with a suitably selected acid such as TFA, HCl, and the like; in an organic solvent such as DCM, THF, and the like; preferably at a temperature in the range of from about 0° C. to about 37° C. Alternatively, wherein PG³ is Boc, the compound of formula (XXII) is de-protected by heating to a temperature in the range of from about 150° C. to about 250° C.; for a period of time in the range of from about 0.5 hours to about 12 hours.

The compound of formula (XXIII) is reacted with a suitably selected base such as sodium bicarbonate, potassium carbonate, and the like; in a suitably selected solvent or mixture thereof such as THF, THF/H₂O, and the like; preferably at a temperature in the range of from about room temperature to about 65° C.; preferably for a period of time in the range of from about 12 hours to about 96 hours; to yield the corresponding compound of formula (XXIV).

The compound of formula (XXIV) is reacted with a suitably selected reducing agent, such as lithium aluminum hydride (LAH), borane, and the like; in an organic solvent, such as THF, ethyl ether, and the like; preferably at a temperature in the range of from about 0° C. to about 65° C.; preferably for a period of time in the range of from about 1 hour to about 24 hours; to yield the corresponding compound of formula (XXV).

The compound of formula (XXV) is reacted with dimethylsulfoxide (DMSO) and a carbodiimide such as 1-ethyl-3-(3-dimethylaminopropyl)carbodiimide (EDCI), dicyclohexylcarbodiimide, and the like; in the presence of pyridinium trifluoroacetate; in an organic solvent, such as methylene chloride, chloroform, and the like; preferably at a temperature in the range of from about 0° C. to about 37° C.; preferably for a period of time in the range of from about 4 hours to about 96 hours; to yield the corresponding compound of formula (XXVI). Alternatively, the compound of formula (XXV) is reacted with a suitably selected oxidizing agent such as Dess-Martin periodinane, oxalyl chloride/DMSO, and the like; and wherein the oxidizing agent is oxalyl chloride/DMSO, in the presence of a tertiary amine base such as TEA, DIPEA, pyridine and the like; in an organic solvent such as methylene chloride, DCM, chloroform, and the like; preferably at a temperature in the range of from about −78° C. to about 25° C.; preferably for a period of time in the range of from about 0.5 hours to about 24 hours; to yield the corresponding compound of formula (XXVI).

The compound of formula (XXVI) is de-benzylated by reacting with 1-chloroethyl chloroformate; in an organic solvent such as acetonitrile, 1,2-dichloroethane, and the like; preferably at a temperature in the range of from about −20° C. to about 80° C.; preferably for a period of time in the range of from about 1 hour to about 48 hours; followed by treatment with a suitably selected aqueous base solution, such as aqueous sodium bicarbonate, aqueous potassium carbonate, aqueous sodium hydroxide, and the like; in an organic solvent such as THF, MeOH, and the like; preferably at a temperature in the range of from about 0° C. to about 60° C.; preferably for a period of time in the range of from about 1 hour to about 24 hours; to yield the corresponding compound of formula (XXVII).

The compound of formula (XXVII) is protected with a suitably selected nitrogen protecting group such as Boc, CBz, and the like, according to known methods, to yield the corresponding compound of formula (XXVIII). For example, the compound of formula (XXVII) is reacted with Boc-anhydride, in THF or a mixture of THF:water; preferably at a temperature in the range of from about 0° C. to about 65° C., to yield the corresponding compound of formula (XXVIII), wherein PG⁴ is Boc.

The compound of formula (XXVIII) is reacted with a suitably selected base such as potassium bis(trimethylsilyl)amide, sodium bis(trimethylsilyl)amide, lithium diisopropylamide, sodium hydride, and the like; in an inert organic solvent, such as THF, ethyl ether, 1,2-dimethoxyethane (DME), and the like; preferably at a temperature in the range of from about −78° C. to about room temperature; preferably for a period of time in the range of from about 0.5 to about 12 hours; and then reacted with N-phenyl-bis(trifluoromethane-sulfonimide); preferably at a temperature in the range of from about −78° C. to about 70° C.; preferably for a period of time in the range of from about 0.5 to about 12 hours; to yield the corresponding compound of formula (XXIX).

The compound of formula (XXIX) is reacted with bis(pinacolato)diboron; in the presence of potassium phenoxide; in the presence of Pd(PPh₃)₂Cl₂; in the presence of PPh₃ (as the phosphine ligand); in an organic solvent such as toluene, benzene, and the like; preferably at a temperature in the range of from about room temperature to about 100° C.; preferably for a period of time in the range of from about 0.5 hours to about 12 hours; to yield the corresponding compound of formula (VIIa).

Compounds of formula (VII) wherein Y is a boronic ester such as

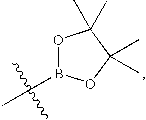

R⁶ is other than hydrogen, and R⁵ is bound at the beta position to the N—R⁷ portion of the R⁰ substituent group may be prepared according to the process outlined in Scheme 5 below.

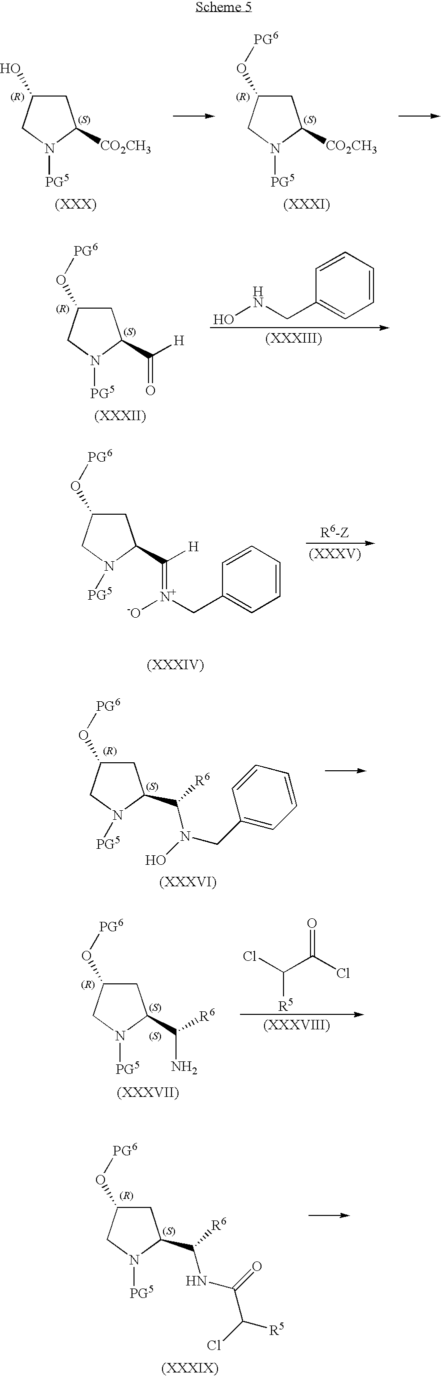

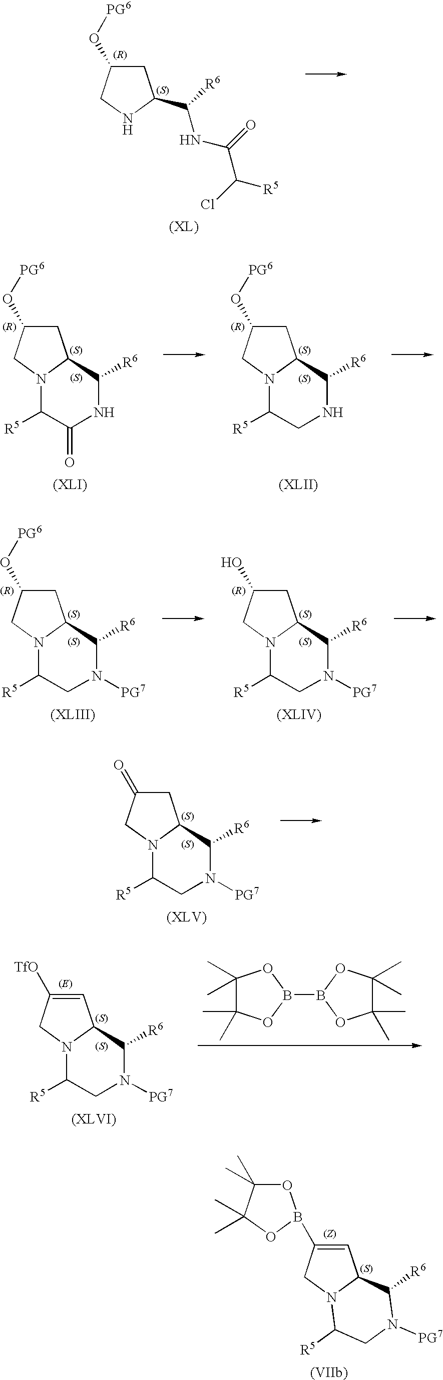

Accordingly, a suitably substituted compound of formula (XXX) wherein PG⁵ is a suitably selected nitrogen protecting group such as Boc, Cbz, and the like, preferably Boc (e.g. N-Boc-4-Hydroxy-L-proline methyl ester) is protected according to known methods, to yield the corresponding compound of formula (XXXI), wherein PG⁶ is the corresponding oxygen protecting group, such as tert-butyldimethylsilyl, triisopropylsilyl, tert-butyldiphenylsilyl, and the like. For example by reacting with tert-butyldimethylsilyl chloride (TBSCl) in the presence of an amine base, such as imidazole, in an organic solvent such as DMF, preferably at a temperature in the range of from about 0° C. to about 60° C.; to yield the corresponding compound of formula (XXXI), wherein PG⁶ is TBS.

The compound of formula (XXXI) is reacted with a suitably selected reducing agent, such as diisobutylaluminum hydride (DIBAL-H), and the like; in an organic solvent such as DCM, toluene, and the like; preferably at a temperature in the range of from about −78° C. to about 0° C.; preferably for a period of time in the range of from about 5 min to about 4 hours; to yield the corresponding compound of formula (XXXII). Alternatively, the compound of formula (XXXI) may be reacted to yield the corresponding compound of formula (XXXII) by first reducing the methyl ester of the compound of formula (XXXI) to the corresponding alcohol according to known methods; followed by oxidation of the alcohol, according to known methods, to yield the corresponding aldehyde, the compound of formula (XXXII).

The compound of formula (XXXII) is reacted with benzylhydroxylamine (a compound of formula (XXXIII); in the presence of a suitably selected drying agent, such as anhydrous MgSO₄, Na₂SO₄, and the like; in an organic solvent such as CH₂Cl₂, CHCl₃, and the like; preferably at a temperature in the range of from about 0° C. to about 37° C.; preferably for a period of time in the range of from about 1 hour to about 24 hours; to yield the corresponding compound of formula (XXXIV).

The compound of formula (XXXIV) is reacted with a suitably substituted compound of formula (XXXV), wherein Z is MgBr, MgCl or Li, a known compound or compound prepared by known methods, in an organic solvent, such as THF, ethyl ether, dimethoxyethane (DME), and the like; preferably at a temperature in the range of from about −78° C. to about room temperature; preferably for a period of time in the range of from about 0.5 hours to about 24 hours; to yield the corresponding compound of formula (XXXVI).

The compound of formula (XXXVI) is reacted with a suitably selected reducing agent such as HCO₂H, H₂ (gas), and the like; in the presence of a suitable catalyst such as palladium on carbon (Pd/C), and the like; in an organic solvent such as methanol, ethanol, and the like; preferably at a temperature in the range of from about 0° C. to about 60° C.; preferably for a period of time in the range of from about 1 hour to about 48 hours; to yield the corresponding compound of formula (XXXVII).

The compound of formula (XXXVII) is reacted with a suitably substituted compound of formula (XXXVIII), a known compound or compound prepared by known methods, in the presence of a base, such as NaHCO₃, K₂CO₃, TEA, and the like; in an organic solvent or mixture thereof such as THF, THF/H₂O, CH₂Cl₂, and the like; preferably at a temperature in the range of from about 0° C. to about 37° C.; preferably for a period of time in the range of from about 0.5 hours to about 15 hours; to yield the corresponding compound of formula (XXXIX).

The compound of formula (XXXIX) is de-protected at the nitrogen, according to known methods, to yield the corresponding compound of formula (XL). For example, wherein PG⁵ is Boc, the compound of formula (XXXIX) is reacted with an acid such as TFA, HCl, H₂SO₄, and the like; in an organic solvent such as such as CH₂Cl₂, THF, and the like; preferably at a temperature in the range of from about 0° C. to about 37° C. In another example, wherein PG⁵ is Boc, the compound of formula (XXXIX) is de-protected by heating to a temperature in the range of from about 150° C. to about 250° C.; for a period of time in the range of from about 0.5 hours to about 12 hours.

The compound of formula (XL) is reacted with a suitably selected base such as NaHCO₃, K₂CO₃, Et₃N, and the like; in and organic solvent or mixture thereof, such as THF, THF/H₂O, CH₂Cl₂, and the like; preferably at a temperature in the range of from about 0° C. to about 37° C.; preferably for a period of time in the range of from about 1 hour to about 48 hours; to yield the corresponding compound of formula (XLI).

The compound of formula (XLI) is reacted with a suitably selected reducing agent such as lithium aluminum hydride (LAH), borane, and the like; in an organic solvent, such as THF, ethyl ether, and the like; preferably at a temperature in the range of from about 0° C. to about 65° C.; preferably for a period of time in the range of from about 1 hour to about 24 hours; to yield the corresponding compound of formula (XLII).

The compound of formula (XLII) is protected at the nitrogen according to known methods, to yield the corresponding compound of formula (XLIII), wherein PG⁷ is a suitably selected nitrogen protecting group, such as Boc, Cbz, and the like. For example, the compound of formula (XLII) may be reacted with Boc-anhydride, in an organic solvent, such as THF or THF/water, preferably at a temperature in the range of from about 0° C. to about 65° C., to yield the corresponding compound of formula (XLIII), wherein PG⁷ is Boc.

The compound of formula (XLIII) is de-protected at the oxygen, according to known methods, to yield the corresponding compound of formula (XLIV). For example, wherein PG⁶ is tert-butyldimethylsilyl (TBS), the compound of formula (XLIII) may be de-protected by reacting with tetrabutylammonium fluoride (TBAF) in an organic solvent, such as THF or 1,4-dioxane; preferably at a temperature in the range of from about 0° C. to about 65° C. Alternatively, wherein PG⁶ is TBS, the compound of formula (XLIII) may be de-protected by reacting with aqueous HF; in an organic solvent, such as acetonitrile or THF; preferably at a temperature in the range of from about 0° C. to about 80° C.

The compound of formula (XLIV) is oxidized by reacting with dimethylsulfoxide (DMSO) and a carbodiimide, such as 1-ethyl-3-(3-dimethylaminopropyl)carbodiimide (EDCI), dicyclohexylcarbodiimide, and the like; in the presence pyridinium trifluoroacetate; in an organic solvent, such as methylene chloride, DCE, chloroform, and the like; preferably at a temperature in the range of from about 0° C. to about 37° C.; preferably for a period of time in the range of from about 4 hours to about 96 hours; to yield the corresponding compound of formula (XLV). Alternatively, the compound of formula (XLIV) is reacted with a suitably selected oxidizing agent such as Dess-Martin periodinane, oxalyl chloride/DMSO, and the like; and wherein the oxidizing agent is oxalyl chloride/DMSO, in the presence of a tertiary amine base such as TEA, DIPEA, pyridine and the like; in an organic solvent such as methylene chloride, DCM, chloroform, and the like; preferably at a temperature in the range of from about −78° C. to about 25° C.; preferably for a period of time in the range of from about 0.5 hours to about 24 hours; to yield the corresponding compound of formula (XLV).

The compound of formula (XLV) is reacted with a suitably selected base such as potassium bis(trimethylsilyl)amide, sodium bis(trimethylsilyl)amide, lithium diisopropylamide, sodium hydride, and the like; in an inert organic solvent, such as THF, ethyl ether, 1,2-dimethoxyethane (DME), and the like; preferably at a temperature in the range of from about −78° C. to about room temperature; preferably for a period of time in the range of from about 0.5 to about 12 hours; and then reacted with N-phenyl-bis(trifluoromethane-sulfonimide); preferably at a temperature in the range of from about −78° C. to about 70° C.; preferably for a period of time in the range of from about 0.5 to about 12 hours; to yield the corresponding compound of formula (XLVI).

The compound of formula (XLVI) is reacted with bis(pinacolato)diboron; in the presence of potassium phenoxide; in the presence of Pd(PPh₃)₂Cl₂; in the presence of PPh₃ (as the phosphine ligand); in an organic solvent such as toluene, benzene, and the like; preferably at a temperature in the range of from about room temperature to about 100° C.; preferably for a period of time in the range of from about 0.5 hours to about 12 hours; to yield the corresponding compound of formula (VIIb).

Compounds of formula (VII) wherein Y is a boronic ester such as

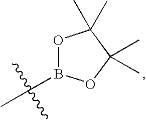

R⁶ is other than hydrogen, and R⁵ is bound at the alpha position to the N—R⁷ portion of the R⁰ substituent group may be prepared according to the process outlined in Scheme 6 below.

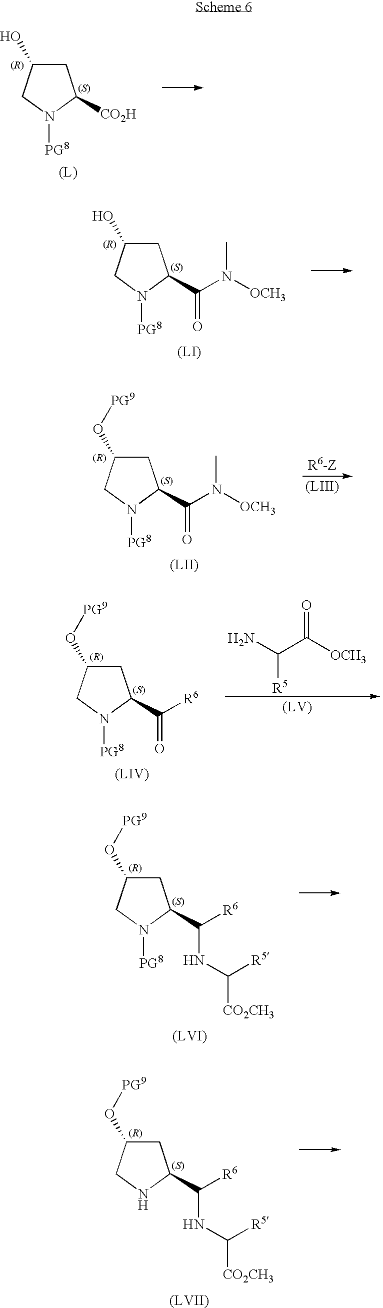

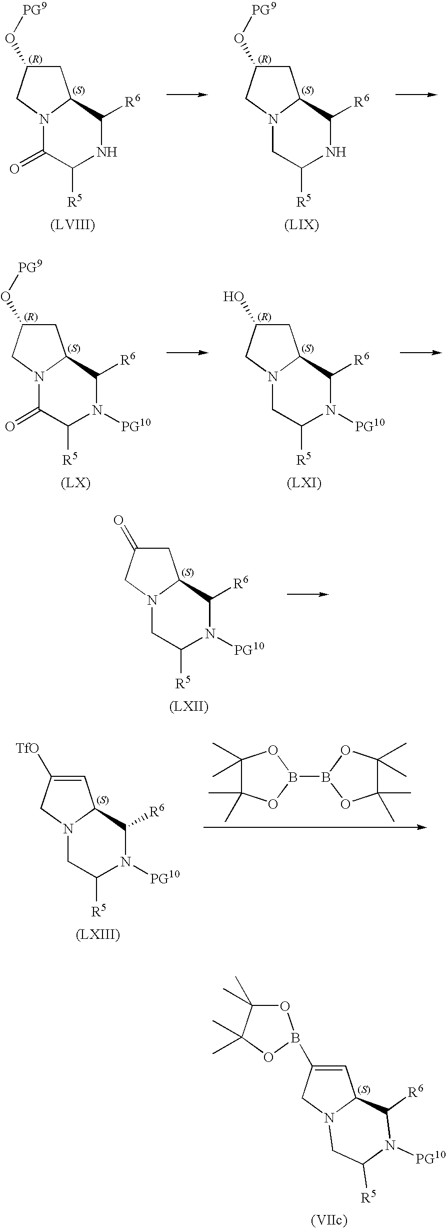

Accordingly, a suitably substituted compound of formula (L), wherein PG⁸ is a suitably selected nitrogen protecting group such as Boc, Cbz, and the like, for example Boc (e.g. N-Boc-4-Hydroxy-L-proline) is reacted with N,O-dimethylhydroxylamine; in the presence of a carbodiimide, such as dicyclohexylcarbodiimide (DCC), 1-ethyl-3-(3-dimethylaminopropyl)carbodiimide (EDCI), and the like; in an organic solvent, such as methylene chloride, DMF, and the like; preferably at a temperature in the range of from about 0° C. to about 37° C.; preferably for a period of time in the range of from about 1 hour to about 48 hours; to yield the corresponding compound of formula (LI).

The compound of formula (LI) is protected at the hydroxy group, according to known methods, to yield the corresponding compound of formula (LI), wherein PG⁹ is a suitably selected oxygen protecting group such as tert-butyldimethylsilyl, triisopropylsilyl, tert-butyldiphenylsilyl, and the like. For example, by reacting with tert-butyldimethylsilyl chloride (TBSCl) in the presence of an amine base, such as imidazole, in an organic solvent such as DCM, DMF, preferably at a temperature in the range of from about 0° C. to about 80° C.; to yield the corresponding compound of formula (LII), wherein PG⁹ is TBS.

The compound of formula (LII) is reacted with a suitably selected compound of formula (LIII), wherein Z is MgBr, MgCl or Li, a known compound or compound prepared by known methods; in an organic solvent, such as ethyl ether, THF, and the like; preferably at a temperature in the range of from about −78° C. to about 40° C.; to yield the corresponding compound of formula (LIV).

The compound of formula (LIV) is reacted with a suitably substituted compound of formula (LV), a known compound or compound prepared by known methods; in the presence of a reducing agent such as NaCNBH₃, sodium triacetoxyborohydride, and the like; in the presence of a suitably selected acid, such as acetic acid, propionic acid, and the like; in an organic solvent, such as MeOH, EtOH, and the like; preferably at a temperature in the range of from about −20° C. to about 60° C.; preferably for a period of time in the range of from about 1 hour to about 48 hours; to yield the corresponding compound of formula (LVI).

The compound of formula (LVI) is nitrogen de-protected according to known methods, to yield the corresponding compound of formula (LVII). For example, wherein PG⁸ is Boc, the compound of formula (LVI) is de-protected by reacting with a suitably selected acid, such as TFA, HCl, and the like; in an organic solvent, such as methylene chloride, DCE, chloroform, and the like; preferably at a temperature in the range of from about 0° C. to about 37° C. Alternatively, wherein PG⁸ is Boc, the compound of formula (LVI) is de-protected by heating to a temperature in the range of from about 150° C. to about 250° C.; preferably for a period of time in the range of from about 0.5 hours to about 12 hours.

The compound of formula (LVII) is reacted with a suitably selected base, such as sodium bicarbonate, potassium carbonate, and the like; in an organic solvent or mixture thereof, such as THF, THF/H₂O, and the like; preferably at a temperature in the range of form about room temperature to about 65° C.; preferably for a period of time in the range of from about 12 hours to about 96 hours; to yield the corresponding compound of formula (LVIII).

The compound of formula (LVIII) is reacted with a suitably selected reducing agent such as lithium aluminum hydride (LAH), borane, and the like; in an organic solvent, such as THF, ethyl ether, 1,4-dioxane, and the like; preferably at a temperature in the range of from about 0° C. to about 65° C.; preferably for a period of time in the range of from about 1 hour to about 24 hours; to yield the corresponding compound of formula (LIX).

The compound of formula (LIX) is protected at the nitrogen, according to known methods, to yield the corresponding compound of formula (LX), wherein PG¹⁰ is the corresponding nitrogen protecting groups. For example, the compound of formula (LIX) is reacted with Boc-anhydride, in an organic solvent or mixture thereof, such as THF, THF/water, at a temperature in the range of from about 0° C. to about 65° C., to yield the corresponding compound of formula (LX), wherein PG¹⁰ is Boc.

The compound of formula (LX) is oxygen de-protected according to known methods, to yield the corresponding compound of formula (LXI). For example, wherein PG⁹ is TBS, the compound of formula (LX) is de-protected by reacting with aqueous HF in an organic solvent, such as acetonitrile, THF, and the like at a temperature in the range of from about 0° C. to about 80° C. Alternatively, wherein PG⁹ is TBS, the compound of formula (LX) is de-protected by reacting with tetrabutylammonium fluoride (TBAF), in an organic solvent, such as THF, ethyl ether, 1,4-dioxane, and the like, at a temperature in the range of from about 0° C. to about 65° C.

The compound of formula (LXI) is oxidized by reacting with dimethylsulfoxide (DMSO) and a carbodiimide, such as 1-ethyl-3-(3-dimethylaminopropyl)carbodiimide (EDCI), dicyclohexylcarbodiimide, and the like; in the presence of pyridinium trifluoroacetate; in an organic solvent, such as methylene chloride, DCE, chloroform, and the like; preferably at a temperature in the range of from about 0° C. to about 37° C.; preferably for a period of time in the range of from about 4 hours to about 96 hours; to yield the corresponding compound of formula (LXII). Alternatively, the compound of formula (LXI) is reacted with a suitably selected oxidizing agent such as Dess-Martin periodinane, oxalyl chloride/DMSO, and the like; and wherein the oxidizing agent is oxalyl chloride/DMSO, in the presence of a tertiary amine base such as TEA, DIPEA, pyridine and the like; in an organic solvent such as methylene chloride, DCE, chloroform, and the like; preferably at a temperature in the range of from about −78° C. to about 25° C.; preferably for a period of time in the range of from about 0.5 hours to about 24 hours; to yield the corresponding compound of formula (LXII).

The compound of formula (LXII) is reacted with a suitably selected base such as potassium bis(trimethylsilyl)amide, sodium bis(trimethylsilyl)amide, lithium diisopropylamide, sodium hydride, and the like; in an inert organic solvent, such as THF, ethyl ether, 1,2-dimethoxyethane (DME), and the like; preferably at a temperature in the range of from about −78° C. to about room temperature; preferably for a period of time in the range of from about 0.5 to about 12 hours; and then reacted with N-phenyl-bis(trifluoromethane-sulfonimide); preferably at a temperature in the range of from about −78° C. to about 70° C.; preferably for a period of time in the range of from about 0.5 to about 12 hours; to yield the corresponding compound of formula (LXIII).

The compound of formula (LXIII) is reacted with bis(pinacolato)diboron; in the presence of potassium phenoxide; in the presence of Pd(PPh₃)₂Cl₂; in the presence of PPh₃ (as the phosphine ligand); in an organic solvent such as toluene, benzene, and the like; preferably at a temperature in the range of from about room temperature to about 100° C.; preferably for a period of time in the range of from about 0.5 hours to about 12 hours; to yield the corresponding compound of formula (VIIc).

One skilled in the art will further recognize that substituting N-(protected)-hydroxy-D-proline for N-(protected)-hydroxy-L-proline in Schemes 4 through 6 above will yield the corresponding compounds

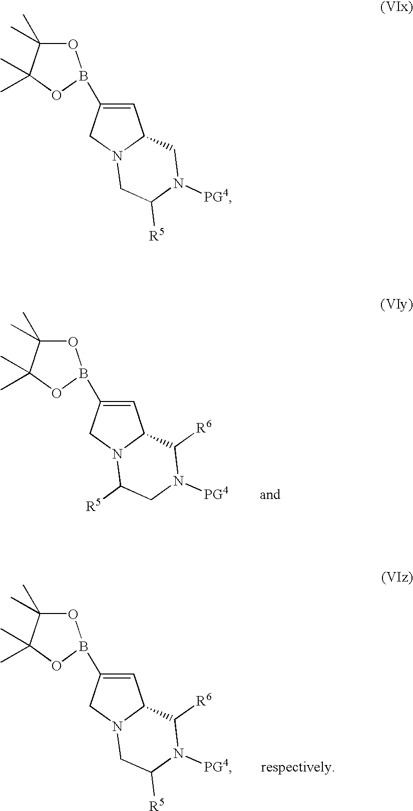

The present invention further comprises pharmaceutical compositions containing one or more compounds of formula (I) with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gel caps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, though other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.01-5000 mg or any range therein, and may be given at a dosage of from about 0.01-100 mg/kg/day, or any range therein, preferably from about 1 to about 50 mg/kg/day, or any range therein. The dosages, however, may be varied depending upon the requirement of the patients, the severity of the condition being treated and the compound being employed. The use of either daily administration or post-periodic dosing may be employed.

Preferably these compositions are in unit dosage forms such as tablets, pills, capsules, powders, granules, sterile parenteral solutions or suspensions, metered aerosol or liquid sprays, drops, ampoules, autoinjector devices or suppositories; for oral, parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.01 to about 1000 mg of the active ingredient of the present invention. The tablets or pills of the novel composition can be coated or otherwise compounded to provide a dosage form yielding the advantage of prolonged action. For example, the tablet or pill can comprise an inner dosage and an outer dosage component, the latter being in the form of an envelope over the former. The two components can be separated by an enteric layer, which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The methods described in the present invention may also be carried out using a pharmaceutical composition comprising any of the compounds as defined herein and a pharmaceutically acceptable carrier. The pharmaceutical composition may contain between about 0.01 mg and 1000 mg of the compound, or any range therein; preferably about 10 to 500 mg of the compound, or any range therein, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms may include suitably flavored suspending or dispersing agents such as the synthetic and natural gums, for example, tragacanth, acacia, methyl-cellulose and the like. For parenteral administration, sterile suspensions and solutions are desired. Isotonic preparations, which generally contain suitable preservatives, are employed when intravenous administration is desired.

The compound of the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine or phosphatidylcholine.

Compounds of the present invention may also be delivered by the use of monoclonal antibodies as individual carriers to which the compound molecules are coupled. The compounds of the present invention may also be coupled with soluble polymers as target able drug carriers. Such polymers can include polyvinylpyrrolidone, pyran copolymer, polyhydroxypropylmethacrylamidephenol, polyhydroxyethylaspartamidephenol, or polyethyleneoxidepolylysine substituted with palmitoyl residue. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

To prepare a pharmaceutical composition of the present invention, a compound of formula (I) as the active ingredient is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

Compounds of this invention may be administered in any of the foregoing compositions and according to dosage regimens established in the art whenever treatment with antimicrobial agents is required.

The daily dosage of the products may be varied over a wide range from 0.01 to 10,000 mg per adult human per day, or any range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250, 500 and 1000 milligrams of the active ingredient for the symptomatic adjustment of the dosage to the patient to be treated. An effective amount of the drug is ordinarily supplied at a dosage level of from about 0.01 mg/kg to about 100 mg/kg of body weight per day, or any range therein. Preferably, the range is from about 0.1 to about 50 mg/kg of body weight per day, or any range therein. More preferably, from about 0.5 to about 25 mg/kg of body weight per day, or any range therein. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trails including first-in-human, dose in the range of and efficacy trials, in healthy patients and/or those suffering from a given disorder, may be completed according to methods well known in the clinical and medical arts.

The following Examples are set forth to aid in the understanding of the invention, and are not intended and should not be construed to limit in any way the invention set forth in the claims which follow thereafter.

In the Examples that follow, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like.

### Precursor Preparation—Heterocyclic Nuclei

All heterocyclic nuclei such as 1-cyclopropyl-1,4-dihydro-6,7-difluoro-8-methoxy-4-oxo-quinoline-3-carboxylic acid, 7-chloro-1-cyclopropyl-6-fluoro-4-oxo-1,4-dihydro-naphthpyridine-3-carboxylic acid, 9,10-difluoro-2,3-dihydro-3-methyl-7-oxo-7H-pyrido[1,2,3-de]-1,4-benzoxazine-6-carboxylic acid, 1-cyclopropyl-1,4-dihydro-6,7-difluoro-4-oxo-quinoline-3-carboxylic acid, 7-chloro-1-(2,4-difluorophenyl)-6-fluoro-4-oxo-1,4-dihydro-naphthyridine-3-carboxylic acid, 1-cyclopropyl-1,4-dihydro-8-difluoromethoxy-6,7-fluoro-4-oxo-quinoline-3-carboxylic acid and 7-bromo-1-cyclopropyl-8-difluoromethoxy-1,4-dihydro-4-oxo-quinoline-3-carboxylic acid were prepared according to literature methods or were purchased from commercial sources.

### Precursor A—Preparation of 7-Trifluoromethylsulfonates

The 7-trifluoromethylsulfonates were prepared as described in Kiely et al, J. Heterocyclic. Chem., 1991, 28,1581.

### Intermediate Compound 1: Ethyl 1-cyclopropyl-1,4-dihydro-6-fluoro-4-oxo-7-[(trifluoromethylsulfonyl)oxy]-quinoline-3-carboxylate

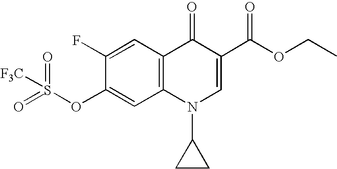

A mixture of ethyl 1-cyclopropyl-1,4-dihydro-6,7-difluoro-4-oxo-quinoline-3-carboxylate (8.0 g, 27.3 mmol) in 1N aqueous NaOH solution (270 mL) was heated to reflux for 8 hours. The resulting solution was cooled to room temperature and the pH of the solution was adjusted to pH˜4 with 6N aqueous HCl solution. The resulting mixture was filtered and the solid was washed with H₂O and diethyl ether, then dried in vacuo to yield 1-cyclopropyl-1,4-dihydro-6-fluoro-7-hydroxy-4-oxo-quinoline-3-carboxylic acid as a light yellow solid.

To a solution of 1-cyclopropyl-1,4-dihydro-6-fluoro-7-hydroxy-4-oxo-quinoline-3-carboxylic acid (7.2 g, 27.4 mmol) in pyridine (100 mL) at 0° C. was slowly added trifluoromethylsulfonic anhydride (11.5 mL, 68.4 mmol). The resulting mixture was allowed to warm to room temperature and then stirred for 2.5 hours. EtOH (60 mL) was added and the resulting solution was stirred for another 45 min, then poured into H₂O. The precipitated solid was collected by filtration, washed with H₂O, dried in vacuo, and purified by silica gel chromatography (2% MeOH in CH₂Cl₂) to yield the title compound as a yellow solid.

MS 424 (M+H).

### Intermediate Compound 2: Ethyl 1-cyclopropyl-1,4-dihydro-6-fluoro-8-methoxy-4-oxo-7-[(trifluoromethyl-sulfonyl)oxy]-quinoline-3-carboxylate

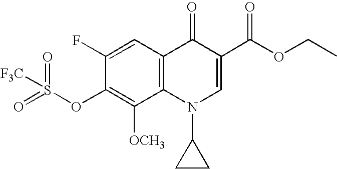

The title compound was prepared in a manner analogous to ethyl 1-cyclopropyl-1,4-dihydro-6-fluoro-4-oxo-7-[(trifluoromethylsulfonyl)oxy]-quinoline-3-carboxylate (Intermediate Compound 1), reacting 1-cyclopropyl-1,4-dihydro-6,7-difluoro-8-methoxy-4-oxo-quinoline carboxylic acid to yield the title compound as a yellow solid.

MS 454 (M+H).

### Intermediate Compound 3: Ethyl 1-cyclopropyl-8-difluoromethoxy-1,4-dihydro-6-fluoro-4-oxo-7-[(trifluoromethylsulfonyl)oxy]-quinoline-3-carboxylate

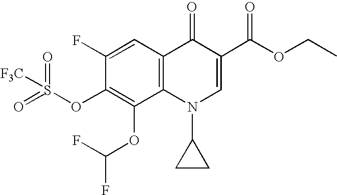

The title compound was prepared in a manner analogous to ethyl 1-cyclopropyl-1,4-dihydro-6-fluoro-4-oxo-7-[(trifluoromethylsulfonyl)oxy]-quinoline-3-carboxylate (Intermediate Compound 1) reacting 1-cyclopropyl-1,4-dihydro-6,7-difluoro-8-difluoromethoxy-4-oxo-quinoline carboxylic acid to yield the title compound as a yellow solid.

MS 454 (M+H).

### Intermediate Compound 4: Ethyl 2,3-dihydro-9-fluoro-3S-methyl-7-oxo-7H-pyrido[1,2,3-de]-10-[(trifluoromethylsulfonyl)oxy]-1,4-benzoxazine-6-carboxylate

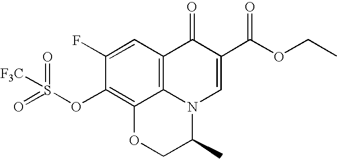

The title compound was prepared in a manner analogous to ethyl 1-cyclopropyl-1,4-dihydro-6-fluoro-4-oxo-7-[(trifluoromethylsulfonyl)oxy]-quinoline-3-carboxylate (Intermediate Example 1) but starting with 9,10-difluoro-2,3-dihydro-3-methyl-7-oxo-7H-pyrido[1,2,3-de]-1,4-benzoxazine-6-carboxylic acid to yield the title compound as a yellow solid.

MS 440 (M+H).

### Intermediate Compound 5: 3S-Methyl-7-(4,4,5,5-tetramethyl-[1,3,2]dioxaborolan-2-yl)-3,4,6,8a-tetrahydro-1H-pyrrolo[1,2S-a]pyrazine-2-carboxylic acid tert-butyl ester

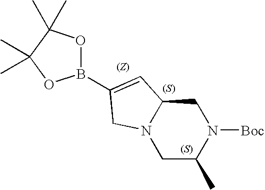

### Step A: 2S-[Benzyl-(1S-methoxycarbonyl-ethyl)-carbamoyl]-4R-hydroxy-pyrrolidine-1-carboxylic acid tert-butyl ester

To a solution of N-Boc-4R-hydroxy-L-proline (10.5 g, 45.5 mmol) in CH₂Cl₂ (250 mL) at room temperature was added N,N′-dicyclohexylcarbodiimide (DCC) (9.7 g, 47.1 mmol). The resulting mixture was stirred for 45 min before a solution of N-benzyl-L-alanine methyl ester (6.5 g, 33.7 mmol) in CH₂Cl₂ (30 mL) was added. The resulting mixture was stirred at room temperature for 24 hours, then filtered and the solution was concentrated. The resulting residue was purified by silica gel chromatography (4% MeOH in CH₂Cl₂) to yield a white foam.

### Step B: 2-Benzyl-7R-hydroxy-3S-methyl-hexahydro-pyrrolo[1,2S-a]pyrazine-1,4-dione

A solution of the product prepared as in Step A above (10.5 g, 25.9 mmol) in CH₂Cl₂ (80 mL) was treated with trifluoroacetic acid (20 mL). The resulting mixture was stirred at room temperature for 5 hours, then concentrated. The resulting residue was diluted with THF (50 mL), basified with sat. aq. NaHCO₃ (50 mL), and stirred at room temperature for 16 hours. The resulting mixture was extracted with CH₂Cl₂ (4×100 mL). The organic layers were combined, washed with brine, dried over MgSO₄, and concentrated. The resulting residue was purified by silica gel chromatography (4% MeOH in CH₂Cl₂) to yield a white foam.

MS 275 (M+H).

### Step C: 2-Benzyl-3S-methyl-octahydro-pyrrolo[1,2S-a]pyrazin-7R-ol

A solution of the product prepared as in Step B above (5.2 g, 19.0 mmol) in THF (250 mL) was treated with LiAlH₄ (3.6 g, 94.7 mmol). The resulting mixture was heated to reflux for 16 hours. The mixture was then cooled to room temperature. To the mixture was added slowly H₂O (3.6 mL), followed by aq. 15% NaOH (3.6 mL), and again H₂O (11 mL). The resulting mixture was filtered and the solution was concentrated. The residue was purified by silica gel chromatography (6% MeOH in CH₂Cl₂ with 0.3% NH₄OH) to yield a brown oil.

### Step D: 2-Benzyl-3S-methyl-hexahydro-pyrrolo[1,2S-a]pyrazin-7-one

A solution of the product prepared as in Step C (4.0 g, 16.3 mmol) in CH₂Cl₂ (150 mL) and DMSO (25 mL) was treated with N-(dimethylaminopropyl)-N′-ethylcarbodiimide hydrochloride (EDCI) (22 g, 114.6 mmol), followed by a solution of pyridinium trifluoroacetate (22 g, 114.6 mmol) in CH₂Cl₂ (50 mL). The resulting mixture was stirred at room temperature for 4 hours. The reaction was quenched with sat. aq. NaHCO₃. The organic layer and the aqueous layer were separated, and the aqueous layer was extracted with CH₂Cl₂ (100 mL). The organic layers were combined, washed with H₂O and brine, dried over MgSO₄, and concentrated. The resulting residue was purified by silica gel chromatography (3% MeOH in CH₂Cl₂) to yield a light yellow oil.

MS 245 (M+H).

### Step E: 3S-Methyl-7-oxo-hexahydro-pyrrolo[1,2S-a]pyrazine-2-carboxylic acid tert-butyl ester

To a solution of the product prepared as in Step D above (1.02 g, 4.2 mmol) in CH₃CN (21 mL) was added 1-chloroethyl chloroformate (1.4 mL, 13 mmol). The resulting mixture was stirred at room temperature for 24 hours, then concentrated. The residue was dissolved in THF (20 mL) and treated with sat. aq. NaHCO₃ (20 mL). The resulting mixture was stirred for 6 hours. The solvent THF was removed in vacuo. The remaining aqueous mixture was washed with ethyl ether, diluted with THF (20 mL), and treated with Boc₂O (1.4 g, 6.4 mmol). The resulting mixture was stirred for 1.5 hours before another portion of Boc₂O (0.7 g, 3.2 mmol) was added. The resulting mixture was stirred for another 1 hour. The mixture was then extracted with CH₂Cl₂ (2×70 mL). The organic layers were combined, washed with H₂O and brine, dried over MgSO₄, and concentrated. The resulting residue was purified by silica gel chromatography (1.5% MeOH in CH₂Cl₂) to yield a light yellow oil.

MS 255 (M+H).

### Step F: 3S-Methyl-7-trifluoromethanesulfonyloxy-3,4,6,8a-tetrahydro-1H-pyrrolo[1,2S-a]pyrazine-2-carboxylic acid tert-butyl ester

To a solution of the product prepared as in Step E above (0.66 g, 2.6 mmol) in THF (20 mL) at −78° C. was added KN(Si(CH₃)₃)₂ (0.5 M solution in toluene, 7 mL, 3.5 mmol). The resulting mixture was stirred at −78° C. for 1 hour before a solution of N-phenyl-bis(trifluoromethane-sulfonimide) (1.37 g, 3.8 mmol) in THF (4 mL) was added. The resulting mixture was kept at −78° C. for 1 hour, then warmed up to 0° C. and stirred for 2 hours. The reaction was quenched with H₂O, and the resulting mixture was extracted with ethyl ether (100 mL). The organic solution was washed with H₂O and brine, dried over MgSO₄, and concentrated. The resulting residue was purified by silica gel chromatography (1% MeOH in CH₂Cl₂) to yield a brown oil.

### Step G: 3S-Methyl-7-(4,4,5,5-tetramethyl-[1,3,2]dioxaborolan-2-yl)-3,4,6,8a-tetrahydro-1H-pyrrolo[1,2S-a]pyrazine-2-carboxylic acid tert-butyl ester

A mixture of the product prepared as in Step F above (0.74 g, 1.9 mmol), potassium phenoxide (0.38 g, 2.9 mmol), bis(pinacolato)diboron (0.536 g, 2.1 mmol), PPh₃ (30 mg, 0.11 mmol), and Pd(PPh₃)₂Cl₂ (40 mg, 0.057 mmol) in toluene (20 mL) was heated at 55° C. for 1 hour. The resulting mixture was allowed to cool to room temperature, then diluted with hexane (60 mL) and filtered. The resulting solution was concentrated to yield the title compound, which was used in the next step without further purification.

MS 365 (M+H).

### Intermediate Compound 6: 3R-Methyl-7-(4,4,5,5-tetramethyl-[1,3,2]dioxaborolan-2-yl)-3,4,6,8a-tetrahydro-1H-pyrrolo[1,2S-a]pyrazine-2-carboxylic acid tert-butyl ester

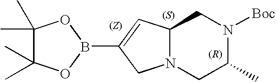

The title compound was prepared in a manner analogous to Intermediate Compound 5 above, substituting N-benzyl-D-alanine methyl ester in place of N-benzyl-L-alanine methyl ester.

MS 365 (M+H).

### Intermediate Compound 7: 7-(4,4,5,5-Tetramethyl-[1,3,2]dioxaborolan-2-yl)-3,4,6,8a-tetrahydro-1H-pyrrolo[1,2S-a]pyrazine-2-carboxylic acid tert-butyl ester

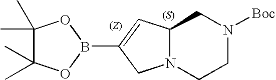

The title compound was prepared in a manner analogous to Intermediate Compound 5 above, substituting N-benzyl-glycine ethyl ester in place of N-benzyl-L-alanine methyl ester.

MS 351 (M+H).

### Intermediate Compound 8: 2-Benzyl-3S-methyl-7-(4,4,5,5-tetramethyl-[1,3,2]dioxaborolan-2-yl)-1,2,3,4,6,8a-hexahydro-pyrrolo[1,2S-a]pyrazine

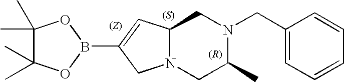

### Step A: Trifluoro-methanesulfonic acid 2-benzyl-3-methyl-1,2,3,4,6,8a-hexahydro-pyrrolo[1,2S-a]pyrazin-7-yl ester

To a solution of the compound prepared as in Intermediate 5, Step D (1.05 g, 4.3 mmol) in THF (40 mL) at −78° C. was added KN(Si(CH₃)₃)₂ (0.5 M solution in toluene, 11 mL, 5.5 mmol). The resulting mixture was stirred at −78° C. for 1 hour before a solution of N-phenyl-bis(trifluoromethane-sulfonimide) (2.3 g, 6.4 mmol) in THF (10 mL) was added. The resulting mixture was then kept at −78° C. for 1 hour, then warmed up to 0° C. and then stirred for 3 hours. The reaction was quenched with H₂O, and the resulting mixture was extracted with ethyl ether (100 mL). The organic solution was washed with H₂O and brine, dried over MgSO₄, and concentrated. The resulting residue was purified by silica gel chromatography (20% EtOAc in hexane) yielded a yellow oil.

### Step B: 2-Benzyl-3S-methyl-7-(4,4,5,5-tetramethyl-[1,3,2]dioxaborolan-2-yl)-1,2,3,4,6,8a-hexahydro-pyrrolo[1,2S-a]pyrazine

A mixture of the product prepared as in Step A above (0.34 g, 0.9 mmol), potassium phenoxide (0.18 g, 1.36 mmol), bis(pinacolato)diboron (0.25 g, 0.98 mmol), PPh₃ (19 mg, 0.07 mmol), and Pd(PPh₃)₂Cl₂ (19 mg, 0.027 mmol) in toluene (9 mL) was heated at 55° C. for 2 hours. The resulting mixture was allowed to cool to room temperature, then diluted with hexane (50 mL) and filtered. The solution was concentrated to yield the title compound, which was used in the next step without further purification.

MS 355 (M+H).

### Intermediate Compound 9: 2-Benzyl-3R-methyl-7-(4,4,5,5-tetramethyl-[1,3,2]dioxaborolan-2-yl)-1,2,3,4,6,8a-hexahydro-pyrrolo[1,2S-a]pyrazine

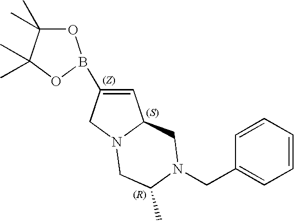

The title compound was prepared in a manner analogous to Intermediate Compound 8 above, substituting N-benzyl-D-alanine methyl ester in place of N-benzyl-L-alanine methyl ester.

MS 355 (M+H).

### Intermediate Compound 10: 2-Benzyl-7-(4,4,5,5-tetramethyl-[1,3,2]dioxaborolan-2-yl)-1,2,3,4,6,8a-hexahydro-pyrrolo[1,2S-a]pyrazine

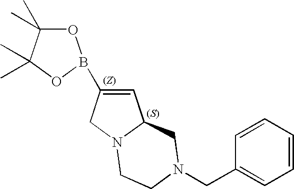

The title compound was prepared in a manner analogous to Intermediate Compound 8, above substituting N-benzyl-glycine ethyl ester in place of N-benzyl-L-alanine methyl ester.

MS 341 (M+H).

### Intermediate Compound 11: 2-Benzyl-3R-tert-butoxymethyl-7-(4,4,5,5-tetramethyl-[1,3,2]dioxaborolan-2-yl)-1,2,3,4,6,8a-hexahydro-pyrrolo[1,2S-a]pyrazine

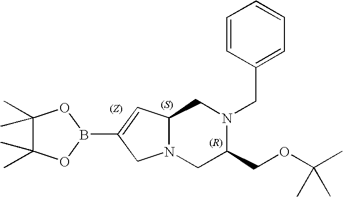

The title compound was prepared in a manner analogous to Intermediate Compound 8, above substituting N-benzyl-O-tert-butyl-L-serine methyl ester in place of N-benzyl-L-alanine methyl ester.

MS 427 (M+H).

### Intermediate Compound 12: 1S-Methyl-7-(4,4,5,5-tetramethyl-[1,3,2]dioxaborolan-2-yl)-3,4,6,8a-tetrahydro-1H-pyrrolo[1,2S-a]pyrazine-2-carboxylic acid tert-butyl ester

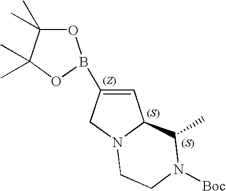

### Step A: 4-(tert-Butyl-dimethyl-silanyloxy)-pyrrolidine-1,2-dicarboxylic acid 1-tert-butyl ester 2S-methyl ester

A solution of N-Boc-4R-hydroxy-L-proline methyl ester (10 g, 40.8 mmol), imidazole (5.6 g, 82.4 mmol), and TBSCl (8.0 g, 53.2 mmol) in DMF (80 mL) was stirred at room temperature for 16 hours. The reaction was quenched with sat. aq. NaHCO₃, and the resulting mixture was extracted with CH₂Cl₂ (2×200 mL). The organic layers were combined, washed with H₂O and brine, dried over MgSO₄, and concentrated. The resulting residue was purified by silica gel chromatography (2% MeOH in CH₂Cl₂) to yield a colorless oil.

MS 360 (M+H).

### Step B: 4R-(tert-Butyl-dimethylsilanyloxy)-2S-[(Z)-[oxido(phenylmethyl)imino]methyl]-pyrrolidine-1-carboxylic acid tert-butyl ester

To a solution of the product prepared as in Step A above (7.2 g, 20.0 mmol) in toluene (150 mL) at −78° C. was added DIBAL-H (1.0 M in hexane, 50 mL, 50 mmol). The resulting mixture was stirred at −78° C. for 30 min, then quenched with MeOH (10 mL) and 1N NaOH aqueous solution. The resulting mixture was warmed up to room temperature and extracted with EtOAc (2×150 mL). The organic layers were combined, washed with H₂O and brine, dried over MgSO₄, and concentrated to yield 4R-(tert-butyl-dimethyl-silanyloxy)-2S-formyl-pyrrolidine-1-carboxylic acid tert-butyl ester as a residue.

The residue (20.0 mmol) was dissolved in CH₂Cl₂ (200 mL) and treated with N-benzylhydroxylamine (2.46g, 20 mmol) and MgSO₄ (4.8 g, 40.0 mmol). The resulting mixture was stirred at room temperature for 6 hours, then filtered and the solution concentrated. The resulting residue was purified by silica gel chromatography (20% EtOAc in hexane) to yield a white solid.

MS 435 (M+H).

### Step C: 2S-[1S-(Benzyl-hydroxy-amino)-ethyl]-4-(tert-butyl-dimethyl-silanyloxy)-pyrrolidine-1-carboxylic acid tert-butyl ester

A solution of the product prepared as in Step B above (7.5 g, 17.3 mmol) in THF (300 mL) was treated with CH₃MgBr (3.0 M in ether, 11.5 mL, 34.5 mmol) at −50° C. The resulting mixture was stirred at −50° C. for 4 hours, then quenched with H₂O. The resulting mixture was then extracted with EtOAc (2×200 mL). The organic layers were combined, washed with H₂O and brine, dried over MgSO₄, and concentrated. The resulting residue was purified by silica gel chromatography (15% EtOAc in hexane) to yield a colorless oil.

MS 451 (M+H).

### Step D: 7R-(tert-Butyl-dimethyl-silanyloxy)-1S-methyl-hexahydro-pyrrolo[1,2S-a]pyrazin-3-one

To a solution of the product prepared as in Step C above (4.6 g, 10.2 mmol) in MeOH (100 mL) was added 10% Pd/C (50% by weight in water) (2.3 g), followed by HCO₂H (5 mL). The resulting mixture was stirred at room temperature under nitrogen for 16 hours. The resulting mixture was then filtered, and concentrated. The resulting residue was dissolved in CH₂Cl₂ (200 mL), washed with aq. 1N NaOH and brine, dried over MgSO₄, and concentrated to yield 2S-(1S-amino-ethyl)-4R-(tert-butyl-dimethyl-silanyloxy)-pyrrolidine-1-carboxylic acid tert-butyl ester as a residue.

A mixture of residue (10.2 mmol) in THF (60 mL) and sat. aq. NaHCO₃ (45 mL) was treated with chloroacetyl chloride (1.22 mL, 15.3 mmol) at room temperature and the resulting mixture stirred for 1 hour. The organic layer and the aqueous layer were separated, and the aqueous layer was extracted with CH₂Cl₂ (150 mL). The organic layers were combined, washed with H₂O and brine, dried over MgSO₄, and concentrated to yield 4-(tert-butyl-dimethyl-silanyloxy)-2S-[1S-(2-chloro-acetylamino)-ethyl]-pyrrolidine-1-carboxylic acid tert-butyl ester as a residue.

To a solution of the residue (10.2 mmol) in CH₂Cl₂ (72 mL) was added trifluoroacetic acid (8 mL). The resulting mixture was stirred at room temperature for 6 hours, then concentrated. The resulting residue was dissolved in CH₂Cl₂, and the solution was washed with sat. aq. NaHCO₃, dried over MgSO₄, and concentrated to yield N-{1S-[4R-(tert-butyl-dimethyl-silanyloxy)-pyrrolidin-2S-yl]-ethyl}-2-chloro-acetamide a residue.

A mixture of the residue (10.2 mmol) in THF (100 mL) and sat. aq. NaHCO₃ (50 mL) was stirred at room temperature for 16 hours. The organic layer and aqueous layer were separated, and the aqueous layer was extracted with CH₂Cl₂ (2×100 mL). The organic layers were combined, washed with H₂O and brine, dried over MgSO₄, and concentrated. The resulting residue was purified by silica gel chromatography (4% MeOH in CH₂Cl₂ with 0.3% NH₄OH) to yield a yellow solid.

MS 285 (M+H).

### Step E: 7R-(tert-Butyl-dimethyl-silanyloxy)-1S-methyl-hexahydro-pyrrolo[1,2S-a]pyrazine-2-carboxylic acid tert-butyl ester

The product prepared as in Step D above (2.6 g, 9.2 mmol) in THF (100 mL) was treated with LiAlH₄ (1.4 g, 36.8 mmol) at 0° C. The resulting mixture was then allowed to warm to room temperature and stirred for 7 hours. To the resulting mixture was slowly added H₂O (1.5 mL), followed by aq. 15% NaOH (1.5 mL) and H₂O (4.5 mL). The resulting mixture was filtered, and the solution was concentrated to yield 7R-(tert-butyl-dimethyl-silanyloxy)-1S-methyl-octahydro-pyrrolo[1,2S-a]pyrazine as a residue.

The residue (9.2 mmol) was dissolved in THF (100 mL) and treated with Boc₂O (2.3 g, 10.6 mmol) at room temperature. The resulting mixture was stirred for 16 hours, then quenched with sat. aq. NaHCO₃. The organic layer and aqueous layer were separated, and the aqueous layer was extracted with CH₂Cl₂ (2×100 mL). The organic layers were combined, washed with brine, dried over MgSO₄, and concentrated. The resulting residue was purified by silica gel chromatography (3% MeOH in CH₂Cl₂ with 0.3% NH₄OH) yielded a colorless oil.

MS 371 (M+H).

### Step F: 1S-Methyl-7-oxo-hexahydro-pyrrolo[1,2S-a]pyrazine-2-carboxylic acid tert-butyl ester

To a solution of the product prepared as in Step E above (2.6 g, 7.0 mmol) in THF (50 mL) was added tetrabutylammonium fluoride (TBAF) (1.0 M solution in THF, 7 mL, 7 mmol). The resulting solution was stirred at room temperature for 5 hours, then concentrated. The residue was diluted with EtOAc (100 mL) and washed with H₂O (20 mL). The aqueous layer was extracted with EtOAc (2×100 mL). The organic layers were combined, washed with brine, dried over Na₂SO₄, and concentrated to yield 7R-hydroxy-1S-methyl-hexahydro-pyrrolo[1,2S-a]pyrazine-2-carboxylic acid tert-butyl ester as a residue.

A solution of the residue (7.0 mmol) in CH₂C₂ (100 mL) and DMSO (10 mL) was treated with EDCI (6.7 g, 34.9 mmol) and pyridinium trifluoroacetate (6.7 g, 34.9 mmol). The resulting mixture was stirred at room temperature for 8 hours, then additional EDCI (3.5 g, 18.2 mmol) and pyridinium trifluoroacetate (3.5 g, 18.2 mmol) were added. The resulting mixture was stirred for 16 hours, then additional EDCI (3.0 g, 15.6 mmol) and pyridinium trifluoroacetate (3.0 g, 15.6 mmol) were added. The resulting mixture was stirred for another 7 hours. The reaction was then quenched with sat. aq. NaHCO₃. The aqueous and organic layers were separated. The aqueous layer was extracted with CH₂Cl₂ (100 mL). The organic layers were combined, washed with H₂O and brine, dried over MgSO₄, and concentrated. The resulting residue was purified by silica gel chromatography (3% MeOH in CH₂Cl₂) to yield a brown oil.

MS 255 (M+H).

### Step G: 1S-Methyl-7-trifluoromethanesulfonyloxy-hexahydro-pyrrolo[1,2S-a]pyrazine-2-carboxylic acid tert-butyl ester

PRD-2968 US and PCT

To a solution of the product prepared as in Step F above (1.57 g, 6.2 mmol) in THF (60 mL) at −78° C. was added KN(Si(CH₃)₃)₂ (0.5 M solution in toluene, 16 mL, 8.0 mmol). The resulting mixture was stirred at −78° C. for 1 hour before a solution of N-phenyl-bis(trifluoromethane-sulfonimide) (3.1 g, 8.7 mmol) in THF (8 mL) was added. The resulting mixture was kept at −78° C. for 1 hour, then warmed up to 0° C. and stirred for 2.5 hours. The reaction was quenched with sat. aq. NaHCO₃, and the resulting mixture was extracted with ethyl ether (2×100 mL). The organic solution was washed with H₂O and brine, dried over MgSO₄, and concentrated. The resulting residue was purified by silica gel chromatography (40% EtOAc in hexane) to yield a red oil.

### Step H: 1S-Methyl-7-(4,4,5,5-tetramethyl-[1,3,2]dioxaborolan-2-yl)-3,4,6,8a-tetrahydro-1H-pyrrolo[1,2S-a]pyrazine-2-carboxylic acid tert-butyl ester

A mixture of the product prepared as in Step G above (0.35 g, 0.91 mmol) with potassium phenoxide (0.18 g, 1.36 mmol), bis(pinacolato)diboron (0.25 g, 0.98 mmol), PPh₃ (19 mg, 0.07 mmol), and Pd(PPh₃)₂Cl₂ (19 mg, 0.027 mmol) in toluene (10 mL) was heated at 55° C. for 3 hours. The resulting mixture was allowed to cool to room temperature, and was then diluted with CH₂Cl₂ (50 mL) and filtered. The resulting solution was concentrated to yield the title compound as a residue, which was used in the next step without further purification.

MS 365 (M+H).

### Intermediate Compound 13: 1R-Methyl-7-(4,4,5,5-tetramethyl-[1,3,2]dioxaborolan-2-yl)-3,4,6,8a-tetrahydro-1H-pyrrolo[1,2S-a]pyrazine-2-carboxylic acid tert-butyl ester

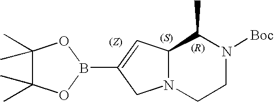

### Step A: 4R-Hydroxy-2S-(methoxy-methyl-carbamoyl)-pyrrolidine-1-carboxylic acid tert-butyl ester

A solution of N-Boc-4R-hydroxy-L-proline (2.3 g, 10.0 mmol) with N,O-dimethylhydroxylamine hydrochloride (1.17g, 12.0 mmol) and Et₃N (1.7 mL, 12.2 mmol) was treated with DCC (2.7 g, 13.1 mmol). The resulting mixture was stirred at room temperature for 16 hours. The resulting mixture was then filtered and the solution was concentrated. The residue was purified by silica gel chromatography (3% MeOH in CH₂Cl₂) to yield a colorless oil.

MS 275 (M+H).

### Step B: 4R-(tert-Butyl-dimethyl-silanyloxy)-2S-(methoxy-methyl-carbamoyl)-pyrrolidine-1-carboxylic acid tert-butyl ester

To a solution of the product prepared as in Step A above (2.2 g, 8.0 mmol) in DMF (20 mL) was added imidazole (1.1 g, 16.2 mmol) followed by TBSCl (1.3 g, 8.6 mmol). The resulting mixture was stirred at room temperature for 18 hours. The resulting mixture was then diluted with CH₂Cl₂ (100 mL), washed with sat. aq. NaHCO₃, H₂O and brine, dried over MgSO₄, and concentrated. The resulting residue was purified by silica gel chromatography (30% EtOAc in hexane) to yield a colorless oil.

MS 389 (M+H).

### Step C: 2S-Acetyl-4R-(tert-butyl-dimethyl-silanyloxy)-pyrrolidine-1-carboxylic acid tert-butyl ester

A solution of the product prepared as in Step B above (1.7 g, 4.4 mmol) in THF (45 mL) was treated with CH₃MgBr (3.0 M solution in ethyl ether, 3 mL, 9.0 mmol) at 0° C. The resulting mixture was allowed to warm to room temperature and stirred for 2 hours before another portion of CH₃MgBr (3 mL, 9.0 mmol) was added. The resulting mixture was kept at room temperature for another 2 hours, then quenched with sat. aq. NH₄Cl. The resulting mixture was extracted with ethyl ether (100 mL). The organic solution was washed with H₂O and brine, dried over MgSO₄, and concentrated. The resulting residue was purified by silica gel chromatography (20% EtOAc/hexane) to yield a colorless oil.

### Step D: Mixture of 4R-(tert-butyl-dimethyl-silanyloxy)-2S-[1R-(methoxycarbonylmethyl-amino)-ethyl]-pyrrolidine-1-carboxylic acid tert-butyl ester

### and 4R-(tert-butyl-dimethyl-silanyloxy)-2S-[1S-(methoxycarbonylmethyl-amino)-ethyl]-pyrrolidine-1-carboxylic acid tert-butyl ester.

To a solution of the product prepared as in Step C above (1.66 g, 4.8 mmol) with glycine methyl ester hydrochloride (0.91 g, 7.3 mmol) and HOAc (1 mL) in MeOH (25 mL) was added NaCNBH₃ (0.60 g, 9.5 mmol). The resulting mixture was stirred at room temperature for 24 hours, then quenched with sat. aq. NaHCO₃. The resulting mixture was extracted with CH₂Cl₂ (2×100 mL). The organic layers were combined, washed with H₂O and brine, dried over MgSO₄, and concentrated to yield a mixture of 4R-(tert-butyl-dimethyl-silanyloxy)-2S-[1r-(methoxycarbonylmethyl-amino)-ethyl]-pyrrolidine-1-carboxylic acid tert-butyl ester and 4R-(tert-butyl-dimethyl-silanyloxy)-2S-[1S-(methoxycarbonylmethyl-amino)-ethyl]-pyrrolidine-1-carboxylic acid tert-butyl ester.

The mixture was dissolved in CH₂Cl₂ (20 mL) and treated with CF₃CO₂H (5 mL). The resulting mixture was stirred at room temperature for 7 hours, then concentrated. The resulting residue was stirred in a mixture of sat. aq. NaHCO₃ (20 mL) and THF (20 mL) for 18 hours. The organic layer and aqueous layer were separated, and the aqueous layer was extracted with CH₂Cl₂ (100 mL). The organic layers were combined, washed with H₂O and brine, dried over MgSO₄, and concentrated. The resulting residue was purified by silica gel chromatography (4% MeOH in CH₂Cl₂ with 0.3% NH₄OH) to yield a residue (the product as a 1:1 mixture of two diastereomers).

MS 285 (M+H).

### Step E: 7R-(tert-Butyl-dimethyl-silanyloxy)-1R-methyl-hexahydro-pyrrolo[1,2S-a]pyrazine-2-carboxylic acid tert-butyl ester and 7R-(tert-Butyl-dimethyl-silanyloxy)-1S-methyl-hexahydro-pyrrolo[1,2S-a]pyrazine-2-carboxylic acid tert-butyl ester

To a solution of the product prepared as in Step D above (0.75 g, 2.6 mmol) in THF (30 mL) at 0° C. was added LiAlH₄ (0.4 g, 10.5 mmol). The resulting mixture was allowed to warm up to room temperature and then stirred for 24 hours. The reaction was then quenched by slow addition of H₂O (0.4 mL) followed by aq. 15% NaOH (0.4 mL) and H₂O (1.2 mL). The resulting mixture was filtered, and the solid was washed with THF (10 mL). The solution was combined and concentrated to yield a residue.

The residue was dissolved in THF (30 mL) and treated with Boc₂O (0.63 g, 2.9 mmol). The resulting mixture was stirred for 6 hours, then diluted with ethyl ether and washed with sat. aq. NaHCO₃. The organic layer was dried over MgSO₄ and concentrated to yield a residue (the product as a 1:1 mixture of two diastereomers). The two diastereomers were separated by silica gel chromatography (3% MeOH in CH₂Cl₂) to yield 7R-(tert-Butyl-dimethyl-silanyloxy)-1R-methyl-hexahydro-pyrrolo[1,2S-a]pyrazine-2-carboxylic acid tert-butyl ester and 7R-(tert-Butyl-dimethyl-silanyloxy)-1S-methyl-hexahydro-pyrrolo[1,2S-a]pyrazine-2-carboxylic acid tert-butyl ester (the same product prepared as in Intermediate Compound 12, step E).

MS 371 (M+H).

### Step F: 1R-Methyl-7-oxo-hexahydro-pyrrolo[1,2S-a]pyrazine-2-carboxylic acid tert-butyl ester

To a solution of the 7R-(tert-Butyl-dimethyl-silanyloxy)-1R-methyl-hexahydro-pyrrolo[1,2S-a]pyrazine-2-caboxylic acid tert-butyl ester prepared as in SETP E above (0.8 g, 2.2 mmol) in CH₃CN (16 mL) was added HF (48% aqueous solution, 1.2 mL). The resulting mixture was stirred at room temperature for 16 hours, then quenched with sat. aq. NaHCO₃. The resulting mixture was extracted with CH₂Cl₂ (2×50 mL). The organic layers were combined, washed with brine, dried over NaSO₄, and concentrated to yield 7R-hydroxy-1R-methyl-hexahydro-pyrrolo[1,2S-a]pyrazine-2-carboxylic acid tert-butyl ester as a residue.

The residue was dissolved in a mixture of CH₂Cl₂ (20 mL) and DMSO (4 mL). To the resulting solution was added EDCI (2.2 g, 11.5 mmol) and pyridinium trifluoroacetate (2.2 g, 11.5 mmol). The resulting mixture was stirred for 4 hours before another portion of EDCI (1.0 g, 5.2 mmol) and pyridinium trifluoroacetate (1.0 g, 5.2 mmol) were added. The resulting mixture was stirred for 16 hours before another portion of EDCI (1.0 g, 5.2 mmol), pyridinium trifluoroacetate (1.0 g, 5.2 mmol) and DMSO (2 mL) were added. The resulting mixture was stirred for another 5 hours and then quenched with sat. aq. NaHCO₃. The resulting mixture was extracted with CH₂Cl₂ (80 mL). The organic layer was washed with H₂O and brine, dried over MgSO₄, and concentrated. The resulting residue was purified by silica gel chromatography (3% MeOH in CH₂Cl₂) to yield a yellow solid.

MS 255 (M+H).

### Step G: 1R-Methyl-7-trifluoromethanesulfonyloxy-3,4,6,8a-tetrahydro-1H-pyrrolo[1,2S-a]pyrazine-2-carboxylic acid tert-butyl ester

A solution of the product prepared as in Step F above (0.4 g, 1.6 mmol) in THF (16 mL) was treated with KN(Si(CH₃)₃)₂ (0.5 M solution in toluene, 4.1 mL, 2.1 mmol) at −78° C. The resulting mixture was kept at −78° C. for 1 hour before a solution of N-phenyl-bis(trifluoromethane-sulfonimide) (0.79 g, 2.2 mmol) in THF (2 mL) was added. The resulting mixture was kept at −78° C. for 1 hour, then warmed up to 0° C. and stirred for 2 hours. The reaction was quenched with sat. aq. NaHCO₃, and the resulting mixture was extracted with ethyl ether (2×50 mL). The organic solution was washed with H₂O and brine, dried over MgSO₄, and concentrated. The resulting residue was purified by silica gel chromatography (20% EtOAc in hexane) to yield a red oil.

### Step H: 1R-Methyl-7-(4,4,5,5-tetramethyl-[1,3,2]dioxaborolan-2-yl)-3,4,6,8a-tetrahydro-1H-pyrrolo[1,2S-a]pyrazine-2-carboxylic acid tert-butyl ester

A mixture of the product prepared as in Step G above (0.29 g, 0.75 mmol) with potassium phenoxide (0.15 g, 1.14 mmol), bis(pinacolato)diboron (0.21 g, 0.83 mmol), PPh₃ (16 mg, 0.06 mmol), and Pd(PPh₃)₂Cl₂ (16 mg, 0.023 mmol) in toluene (8 mL) was heated at 55° C. for 1 hour. The resulting mixture was allowed to cool to room temperature, then diluted with hexane (50 mL) and filtered. The resulting solution was concentrated to yield the title compound as a residue, which was used in the next step without further purification.

MS 365 (M+H).

### Intermediate Compound 14: 1R,3S-Dimethyl-7-(4,4,5,5-tetramethyl-[1,3,2]dioxaborolan-2-yl)-3,4,6,8a-tetrahydro-1H-pyrrolo[1,2S-a]pyrazine-2-carboxylic acid tert-butyl ester

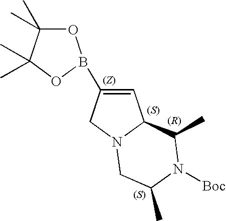

The title compound was prepared in a manner analogous to Intermediate Compound 13, above substituting using L-alanine methyl ester hydrochloride in place of glycine methyl ester hydrochloride.

MS 379 (M+H).

### EXAMPLE 1

### 1-Cyclopropyl-8-difluoromethoxy-4-oxo-7-(1,2,3,4-tetrahydro-pyrrolo[1,2-a]pyrazin-7-yl)-1,4-dihydro-quinoline-3-carboxylic acid (Compound #1)

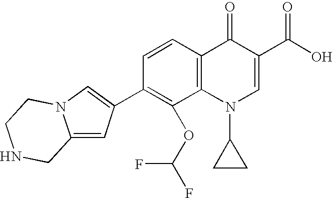

### Step A: 7-(2-tert-Butoxycarbonyl-1,2,3,4,6,8a-hexahydro-pyrrolo[1,2-a]pyrazin-7-yl)-1-cyclopropyl-8-difluoromethoxy-4-oxo-1,4-dihydro-quinoline-3-carboxylic acid ethyl ester

A mixture of Intermediate Compound 7 (1.96 mmol), ethyl 7-bromo-1-cyclopropyl-8-difluoromethoxy-1,4-dihydro-4-oxo-quinoline-3-carboxylate (0.79 g, 1.97 mmol), CsF (1.2 g, 7.89 mmol), and Pd(PPh₃)₄ (136 mg, 0.12 mmol) in THF (40 mL) was heated to reflux for 3 hours. The resulting mixture was allowed to cool to room temperature, diluted with CH₂Cl₂ (100 mL), and filtered. The resulting solution was concentrated and the residue was purified by silica gel chromatography (4% MeOH in CH₂Cl₂ with 0.3% NH₄OH) to yield a yellow solid.

MS 546 (M+H).

### Step B: 7-(2-tert-Butoxycarbonyl-1,2,3,4-tetrahydro-pyrrolo[1,2-a]pyrazin-7-yl)-1-cyclopropyl-8-difluoromethoxy-4-oxo-1,4-dihydro-quinoline-3-carboxylic acid ethyl ester

A mixture of the product from Step A (0.54 g, 0.99 mmol) with Pd/C (10 wt. %, 0.54 g) in MeOH (20 mL) was stirred at room temperature under air for 48 hours. The resulting mixture was filtered and the solution was concentrated. The resulting residue was purified by silica gel chromatography (2% MeOH in CH₂Cl₂ with 0.3% NH₄OH) to yield a yellow solid.

MS 544 (M+H).

### Step C: 1-Cyclopropyl-8-difluoromethoxy-4-oxo-7-(1,2,3,4-tetrahydro-pyrrolo[1,2-a]pyrazin-7-yl)-1,4-dihydro-quinoline-3-carboxylic acid

A solution of the product from Step B (26 mg, 0.048 mmol) in a mixture of THF (1 mL) and MeOH (1 mL) was treated with aq. 1N NaOH (1 mL). The resulting mixture was stirred for 15 min at room temperature, then acidified with aq. 1N HCl to pH=5. The resulting mixture was extracted with EtOAc (2×20 mL). The organic layers were combined, washed with brine, dried over MgSO₄, and concentrated. The resulting residue was dissolved in CH₂Cl₂ (2.5 mL) and treated with 4N HCl in dioxane (0.5 mL). The resulting mixture was stirred for 4 hours, then diluted with ethyl ether (10 mL) and filtered. The resulting solid was washed with ethyl ether and dried in vacuo to yield the title compound as a yellow solid.

MS 416 (M+H).

### EXAMPLE 2

### 7-(2-Benzyl-1,2,3,4,6,8a-hexahydro-pyrrolo[1,2S-a]pyrazin-7-yl)-1-cyclopropyl-8-difluoromethoxy-4-oxo-1,4-dihydro-quinoline-3-carboxylic acid (Compound #2)

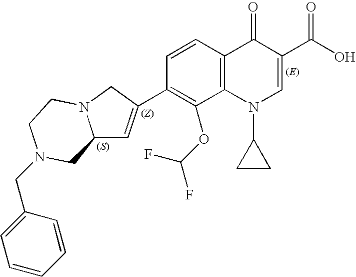

### Step A: 7-(2-Benzyl-1,2,3,4,6,8a-hexahydro-pyrrolo[1,2S-a]pyrazin-7-yl)-1-cyclopropyl-8-difluoromethoxy-4-oxo-1,4-dihydro-quinoline-3-carboxylic acid ethyl ester

A mixture of Intermediate Compound 10 (1.2 mmol), ethyl 7-bromo-1-cyclopropyl-8-difluoromethoxy-1,4-dihydro-4-oxo-quinoline-3-carboxylate (0.5 g, 1.24 mmol), CsF(0.55 g, 3.62 mmol), and Pd(PPh₃)₄ (69 mg, 0.06 mmol) in THF (30 mL) was heated to reflux for 4 hours. The resulting was allowed to cool to room temperature and then diluted with CH₂Cl₂ (100 mL). The resulting mixture was filtered and the solution was concentrated. The resulting residue was purified by silica gel chromatography (5% MeOH in CH₂Cl₂ with 0.3% NH₄OH) to yield a yellow solid.

MS 536 (M+H).

### Step B: 7-(2-Benzyl-1,2,3,4,6,8a-hexahydro-pyrrolo[1,2S-a]pyrazin-7-yl)-1-cyclopropyl-8-difluoromethoxy-4-oxo-1,4-dihydro-quinoline-3-carboxylic acid

A mixture of the product prepared as in Step A above (46 mg, 0.086 mmol) in conc. HCl (1 mL) was heated to reflux for 1 hour. The resulting mixture was allowed to cool to room temperature and concentrated. The resulting residue was suspended in H₂O (2 mL) and filtered. The resulting solid was washed with diethyl ether and dried in vacuo to yield the title compound as a brown solid.

MS 508 (M+H).

### EXAMPLE 3

### 1-Cyclopropyl-8-difluoromethoxy-7-(octahydro-pyrrolo[1,2S-a]pyrazin-7-yl)-4-oxo-1,4-dihydro-quinoline-3-carboxylic acid (Compound #3)

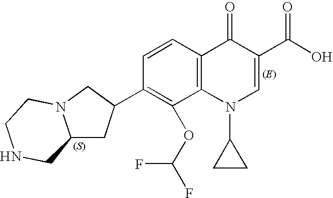

### Step A: 7-(2-Benzyl-1,2,3,4-tetrahydro-pyrrolo[1,2S-a]pyrazin-7-yl)-1-cyclopropyl-8-difluoromethoxy-4-oxo-1,4-dihydro-quinoline-3-carboxylic acid ethyl ester

### and 7-(2-Benzyl-octahydro-pyrrolo[1,2S-a]pyrazin-7-yl)-1-cyclopropyl-8-difluoromethoxy-4-oxo-1,4-dihydro-quinoline-3-carboxylic acid ethyl ester

A mixture of the product prepared as in Step A, Example 2 (180 mg, 0.34 mmol) with Pd/C (10 wt. %, 60 mg) in MeOH (25 mL) was hydrogenated (H₂, 40 psi) for 16 hours. The resulting mixture was filtered and the solution was concentrated. Separation by silica gel chromatography yield 7-(2-Benzyl-1,2,3,4-tetrahydro-pyrrolo[1,2S-a]pyrazin-7-yl)-1-cyclopropyl-8-difluoromethoxy-4-oxo-1,4-dihydro-quinoline-3-carboxylic acid ethyl ester (2% MeOH in CH₂Cl₂ with 0.3% NH₄OH) as a yellow oil;

MS 534 (M+H)

and 7-(2-Benzyl-octahydro-pyrrolo[1,2S-a]pyrazin-7-yl)-1-cyclopropyl-8-difluoromethoxy-4-oxo-1,4-dihydro-quinoline-3-carboxylic acid ethyl ester (4% MeOH in CH₂Cl₂ with 0.3% NH₄OH) as a yellow solid.

MS 538 (M+H)

### Step B: 1-Cyclopropyl-8-difluoromethoxy-7-(octahydro-pyrrolo[1,2S-a]pyrazin-7-yl)-4-oxo-1,4-dihydro-quinoline-3-carboxylic acid ethyl ester

A mixture of 7-(2-Benzyl-octahydro-pyrrolo[1,2S-a]pyrazin-7-yl)-1-cyclopropyl-8-difluoromethoxy-4-oxo-1,4-dihydro-quinoline-3-carboxylic acid ethyl ester prepared as in Step A above, (85 mg, 0.16 mmol) with Pd/C (10 wt. %, 85 mg) and HCO₂H (0.5 mL) in MeOH (9.5 mL) was stirred under nitrogen for 1 hour. The resulting mixture was filtered and the solution was concentrated. The resulting residue was purified by silica gel chromatography (10% MeOH in CH₂Cl₂ with 0.3% NH₄OH) to yield a yellow solid.

MS 448 (M+H).

### Step C: 1-Cyclopropyl-8-difluoromethoxy-7-(octahydro-pyrrolo[1,2S-a]pyrazin-7-yl)-4-oxo-1,4-dihydro-quinoline-3-carboxylic acid

The product prepared as in Step B above (60 mg, 0.13 mmol) was treated with conc. HCl (1 mL). The resulting mixture was heated to reflux for 2 hours, then allowed to cool to room temperature and concentrated. The resulting residue was suspended in dioxane (3 mL) and filtered. The resulting solid was washed with ethyl ether and dried in vacuo to yield the title compound.

MS 420 (M+H).

### EXAMPLE 4

### 1-Cyclopropyl-8-difluoromethoxy-7-(2-methyl-1,2,3,4-tetrahydro-pyrrolo[1,2-a]pyrazin-7-yl)-4-oxo-1,4-dihydro-quinoline-3-carboxylic acid (Compound #4)

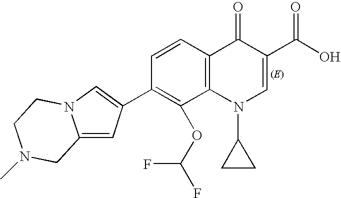

### Step A: 1-Cyclopropyl-8-difluoromethoxy-7-(2-methyl-1,2,3,4-tetrahydro-pyrrolo[1,2-a]pyrazin-7-yl)-4-oxo-1,4-dihydro-quinoline-3-carboxylic acid ethyl ester

A mixture of 7-(2-Benzyl-1,2,3,4-tetrahydro-pyrrolo[1,2-a]pyrazin-7-yl)-1-cyclopropyl-8-difluoromethoxy-4-oxo-1,4-dihydro-quinoline-3-carboxylic acid ethyl ester prepared as in Step A, Example 3 (36 mg, 0.068 mmol) with Pd/C (10 wt. %, 20 mg) and HCO₂H (0.25 mL) in MeOH (4.75 mL) was stirred under nitrogen for 18 hours. The resulting mixture was filtered and the solution was concentrated. The resulting residue was purified by silica gel chromatography (3% MeOH in CH₂Cl₂ with 0.3% NH₄OH) to yield the product.

MS 458 (M+H).

### Step B: 1-Cyclopropyl-8-difluoromethoxy-7-(2-methyl-1,2,3,4-tetrahydro-pyrrolo[1,2-a]pyrazin-7-yl)-4-oxo-1,4-dihydro-quinoline-3-carboxylic acid

The product from Step A above (15 mg, 0.033 mmol) was treated with conc. HCl (1 mL). The resulting mixture was heated to reflux for 2 hours, then cooled to room temperature and concentrated. The resulting residue was suspended in ethyl ether and filtered. The solid was washed with diethyl ether and dried in vacuo to yield the title compound as a brown solid.

MS 430 (M+H).

### EXAMPLE 5

### 1-Cyclopropyl-8-difluoromethoxy-7-(2-methyl-octahydro-pyrrolo[1,2s-a]pyrazin-7-yl)-4-oxo-1,4-dihydro-quinoline-3-carboxylic acid (Compound #5)

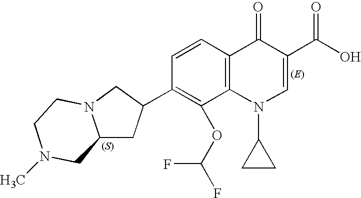

### Step A: 1-Cyclopropyl-8-difluoromethoxy-7-(2-methyl-octahydro-pyrrolo[1,2s-a]pyrazin-7-yl)-4-oxo-1,4-dihydro-quinoline-3-carboxylic acid ethyl ester

A mixture of 7-(2-Benzyl-octahydro-pyrrolo[1,2S-a]pyrazin-7-yl)-1-cyclopropyl-8-difluoromethoxy-4-oxo-1,4-dihydro-quinoline-3-carboxylic acid ethyl ester prepared as in Step A, Example 3 (50 mg, 0.093 mmol) with Pd/C (10 wt. %, 20 mg) and HCO₂H (0.5 mL) in MeOH (9.5 mL) was stirred under nitrogen for 78 hours. The resulting mixture was filtered and the solution was concentrated. The resulting residue was purified by silica gel chromatography (8% MeOH in CH₂Cl₂ with 0.3% NH₄OH) to yield the product.

MS 462 (M+H).

### Step B: 1-Cyclopropyl-8-difluoromethoxy-7-(2-methyl-octahydro-pyrrolo[1,2S-a]pyrazin-7-yl)-4-oxo-1,4-dihydro-quinoline-3-carboxylic acid

The product from Step A above (21 mg, 0.046 mmol) was treated with conc. HCl (1 mL). The resulting mixture was heated to reflux for 2 hours, then allowed to cool to room temperature and concentrated. The resulting residue was suspended in ethyl ether and filtered. The resulting solid was dried in vacuo to yield the title compound as a light brown solid.

MS 434 (M+H).

### EXAMPLE 6

### 1-Cyclopropyl-8-difluoromethoxy-7-(3S-methyl-octahydro-pyrrolo[1,2S-a]pyrazin-7-yl)-4-oxo-1,4-dihydro-quinoline-3-carboxylic acid (Compound #6)

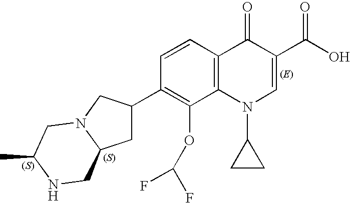

### Step A: 7-(2-tert-Butoxycarbonyl-3S-methyl-1,2,3,4,6,8a-hexahydro-pyrrolo[1,2S-a]pyrazin-7-yl)-1-cyclopropyl-8-difluoromethoxy-4-oxo-1,4-dihydro-quinoline-3-carboxylic acid ethyl ester

A mixture of Intermediate Compound 5 (0.82 mmol), ethyl 7-bromo-1-cyclopropyl-8-difluoromethoxy-1,4-dihydro-4-oxo-quinoline-3-carboxylate (0.33 g, 0.82 mmol), CsF (0.38 g, 2.5 mmol), and Pd(PPh₃)₄ (57 mg, 0.049 mmol) in THF (20 mL) was heated to reflux for 21 hours. The resulting mixture was allowed to cool to room temperature and then diluted with CH₂Cl₂ (100 mL). The resulting mixture was filtered and the solution was concentrated. The resulting residue was purified by silica gel chromatography (3% MeOH in CH₂Cl₂ with 0.3% NH₄OH) to yield the product as a brown oil.

MS 560 (M+H).

### Step B: 7-(2-tert-Butoxycarbonyl-3S-methyl-octahydro-pyrrolo[1,2S-a]pyrazin-7-yl)-1-cyclopropyl-8-difluoromethoxy-4-oxo-1,4-dihydro-quinoline-3-carboxylic acid ethyl ester

A mixture of the product from Step A above (175 mg, 0.31 mmol) with Pd/C (10 wt. %, 45 mg) was treated with H₂ (40 psi) for 15 hours. The resulting mixture was filtered and the solution was concentrated. The resulting residue was purified by silica gel chromatography (2% MeOH in CH₂Cl₂ with 0.3% NH₄OH) to yield the product as a yellow solid.

### Step C: 1-Cyclopropyl-8-difluoromethoxy-7-(3S-methyl-octahydro-pyrrolo[1,2S-a]pyrazin-7-yl)-4-oxo-1,4-dihydro-quinoline-3-carboxylic acid

The product from Step B above (55 mg, 0.098 mmol) was treated with conc. HCl (1 mL). The resulting mixture was heated to reflux for 2 hours, then allowed to cool to room temperature and concentrated. The resulting residue was suspended in ethyl ether and filtered. The resulting solid was dried in vacuo to yield the title compound as a light brown solid.

MS 434 (M+H).

### EXAMPLE 7

### 1-Cyclopropyl-8-difluoromethoxy-7-(3S-methyl-1,2,3,4-tetrahydro-pyrrolo[1,2-a]pyrazin-7-yl)-4-oxo-1,4-dihydro-quinoline-3-carboxylic acid (Compound #7)

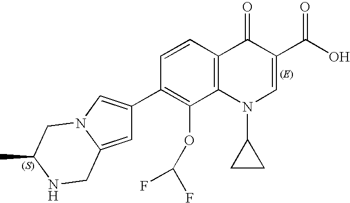

Compound 7 was prepared in a manner analogous to Compound 1, reacting Intermediate Compound 5 instead of Intermediate Compound 7.

MS 430 (M+H).

### EXAMPLE 8

### 1-Cyclopropyl-8-difluoromethoxy-7-(1S-methyl-1,2,3,4-tetrahydro-pyrrolo[1,2-a]pyrazin-7-yl)-4-oxo-1,4-dihydro-quinoline-3-carboxylic acid (Compound #8)

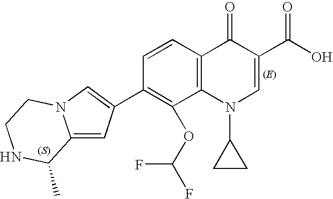

Compound 8 was prepared in a manner analogous to Compound 1, reacting Intermediate Compound 12 instead of Intermediate Compound 7.

MS 430 (M+H).

### EXAMPLE 9

### 1-Cyclopropyl-8-difluoromethoxy-7-(1R-methyl-1,2,3,4-tetrahydro-pyrrolo[1,2-a]pyrazin-7-yl)-4-oxo-1,4-dihydro-quinoline-3-carboxylic acid (Compound #9)

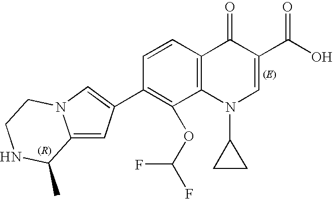

Compound 9 was prepared in a manner analogous to Compound 1 reacting Intermediate Compound 13 instead of Intermediate Compound 7.

MS 430 (M+H).

### EXAMPLE 10

### 1-Cyclopropyl-8-difluoromethoxy-7-[2S-(2-methylamino-propionyl)-1,2,3,4-tetrahydro-pyrrolo[1,2-a]pyrazin-7-yl]-4-oxo-1,4-dihydro-quinoline-3-carboxylic acid (Compound #10)

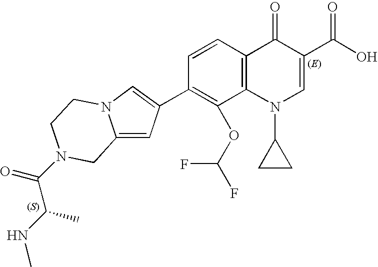

### Step A: 1-Cyclopropyl-8-difluoromethoxy-4-oxo-7-(1,2,3,4-tetrahydro-pyrrolo[1,2-a]pyrazin-7-yl)-1,4-dihydro-quinoline-3-carboxylic acid ethyl ester

A solution of the product prepared as in Step B, Example 1 (100 mg, 0.18 mmol) in CH₂Cl₂ (5 mL) was treated with TFA (1 mL). The resulting mixture was stirred for 3 hours, then concentrated. The residue was diluted with CH₂Cl₂ (50 mL), washed with sat. aq. NaHCO₃, dried over anhydrous Na₂SO₄, and concentrated to yield a residue.

### Step B: 7-{2S-[2-(tert-Butoxycarbonyl-methyl-amino)-propionyl]-1,2,3,4-tetrahydro-pyrrolo[1,2-a]pyrazin-7-yl}-1-cyclopropyl-8-difluoromethoxy-4-oxo-1,4-dihydro-quinoline-3-carboxylic acid ethyl ester

The product from Step A above (0.18 mmol) was dissolved in CH₂Cl₂ (5 mL) and treated with N-Boc-N-methyl-L-alanine (75 mg, 0.37 mmol) followed by DCC (76 mg, 0.37 mmol). The resulting mixture was stirred for 16 hours. The resulting mixture was then filtered and the solution was concentrated. The resulting residue was purified by silica gel chromatography (3% MeOH in CH₂Cl₂) to yield a residue.

### Step C: 1-Cyclopropyl-8-difluoromethoxy-7-[2-(2-methylamino-propionyl)-1,2,3,4-tetrahydro-pyrrolo[1,2-a]pyrazin-7-yl]-4-oxo-1,4-dihydro-quinoline-3-carboxylic acid

A solution of the product prepared in Step B above (50 mg, 0.08 mmol) in MeOH (1 mL) and THF (1 mL) was treated with aq. 1N NaOH (1 mL). The resulting mixture was stirred at room temperature for 15 min, then acidified to pH 4 with aq. 1N HCl. The resulting mixture was extracted with EtOAc (2×20 mL). The organic layers were combined, dried over anhydrous Na₂SO₄, and concentrated. The resulting residue was dissolved in CH₂Cl₂ (2 mL) and treated with 4N HCl in dioxane (0.5 mL). The resulting mixture was stirred for 3 hours, then diluted with diethyl ether (5 mL) and filtered. The resulting solid was washed with diethyl ether and dried in vacuo to yield the title compound as a purple-red solid.

MS 501 (M+H).

### EXAMPLE 11

### 1-Cyclopropyl-6-fluoro-8-methoxy-7-(3S-methyl-1,2,3,4-tetrahydro-pyrrolo[1,2-a]pyrazin-7-yl)-4-oxo-1,4-dihydro-quinoline-3-carboxylic acid (Compound #11)

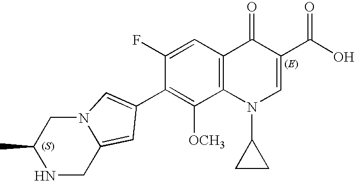

Compound 11 was prepared in a manner analogous to Compound 1, reacting Intermediate Compound 5 in place of Intermediate Compound 7 and using ethyl 1-cyclopropyl-1,4-dihydro-6-fluoro-8-methoxy-4-oxo-7-[(trifluoromethyl-sulfonyl)oxy]-quinoline-3-carboxylate (Intermediate Compound 2) in place of ethyl 7-bromo-1-cyclopropyl-8-difluoromethoxy-1,4-dihydro-4-oxo-quinoline-3-carboxylate.

MS 412 (M+H).

### EXAMPLE 12

### 1-Cyclopropyl-6-fluoro-8-methoxy-7-(3R-methyl-1,2,3,4-tetrahydro-pyrrolo[1,2-a]pyrazin-7-yl)-4-oxo-1,4-dihydro-quinoline-3-carboxylic acid (Compound #12)

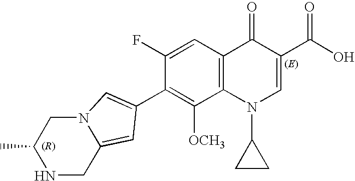

Compound 12 was prepared in a manner analogous to Compound 1, reacting Intermediate Compound 6 in place of Intermediate Compound 7 and using ethyl 1-cyclopropyl-1,4-dihydro-6-fluoro-8-methoxy-4-oxo-7-[(trifluoromethyl-sulfonyl)oxy]-quinoline-3-carboxylate (Intermediate Compound 2) in place of ethyl 7-bromo-1-cyclopropyl-8-difluoromethoxy-1,4-dihydro-4-oxo-quinoline-3-carboxylate.

MS 412 (M+H).

### EXAMPLE 13

### 1-Cyclopropyl-7-(2,3S-dimethyl-1,2,3,4-tetrahydro-pyrrolo[1,2-a]pyrazin-7-yl)-6-fluoro-8-methoxy-4-oxo-1,4-dihydro-quinoline-3-carboxylic acid (Compound #13)

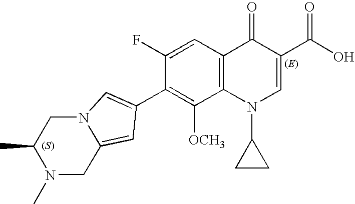

### Step A: 7-(2-Benzyl-3S-methyl-1,2,3,4,6,8a-hexahydro-pyrrolo[1,2s-a]pyrazin-7-yl)-1-cyclopropyl-6-fluoro-8-methoxy-4-oxo-1,4-dihydro-quinoline-3-carboxylic acid ethyl ester

A mixture of Intermediate Compound 8 (0.9 mmol), ethyl 1-cyclopropyl-1,4-dihydro-6-fluoro-8-methoxy-4-oxo-7-[(trifluoromethyl-sulfonyl)oxy]-quinoline-3-carboxylate (Intermediate Compound 2, 0.33 g, 0.73 mmol), CsF (0.41 g, 2.70 mmol), and Pd(PPh₃)₄ (42 mg, 0.036 mmol) in THF (20 mL) was heated to reflux for 7.5 hours. The resulting mixture was allowed to cool to room temperature and then diluted with CH₂Cl₂. The resulting mixture was filtered and the solution was concentrated. The resulting residue was purified by silica gel chromatography (4% MeOH in CH₂Cl₂ with 0.3% NH₄OH) to yield the product as a brown oil.

MS 532 (M+H).

### Step B: 1-Cyclopropyl-7-(2,3S-dimethyl-1,2,3,4-tetrahydro-pyrrolo[1,2-a]pyrazin-7-yl)-6-fluoro-8-methoxy-4-oxo-1,4-dihydro-quinoline-3-carboxylic acid ethyl ester

The product from Step A above (60 mg, 0.11 mmol) was dissolved in MeOH (10 mL) and treated with Pd/C (10 wt. %, 60 mg). The resulting mixture was stirred at room temperature under air for 16 hours. To the resulting mixture was then added HCO₂H (0.5 mL) and Pd/C (10 wt. %, 60 mg) and the resulting mixture stirred under nitrogen for another 2 hours. The resulting mixture was then filtered and the solution was concentrated. The resulting residue was purified by silica gel chromatography (4% MeOH in CH₂Cl₂) to yield the product.

MS 454 (M+H).

### Step C: 1-Cyclopropyl-7-(2,3S-dimethyl-1,2,3,4-tetrahydro-pyrrolo[1,2-a]pyrazin-7-yl)-6-fluoro-8-methoxy-4-oxo-1,4-dihydro-quinoline-3-carboxylic acid

To a solution of the product from Step B above (15 mg) in MeOH (1 mL) and THF (1 mL) was added aq. 1N NaOH (1 mL). The resulting mixture was heated at 55° C. for 2 hours, then cooled to room temperature and acidified with aq. 1N HCl to pH 4. The resulting mixture was extracted with EtOAc. The organic solution was washed with brine, dried over anhydrous NaSO₄, and concentrated. The resulting residue was dissolved in MeOH (0.5 mL) and diluted with diethyl ether. The resulting solid was collected by filtration and dried in vacuo to yield the title compound as a light brown solid.

MS 426 (M+H).

### EXAMPLE 14

### 1-Cyclopropyl-7-(2,3R-dimethyl-1,2,3,4-tetrahydro-pyrrolo[1,2-a]pyrazin-7-yl)-6-fluoro-8-methoxy-4-oxo-1,4-dihydro-quinoline-3-carboxylic acid (Compound #14)

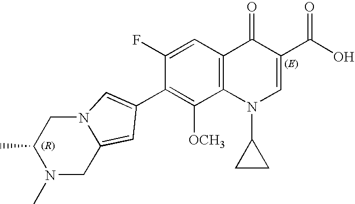

Compound 14 was prepared in a manner analogous to Compound 13, reacting Intermediate Compound 9 in place of Intermediate Compound 8.

MS 426 (M+H).

### EXAMPLE 15

### 1-Cyclopropyl-6-fluoro-7-(3R-hydroxymethyl-2-methyl-1,2,3,4-tetrahydro-pyrrolo[1,2-a]pyrazin-7-yl)-8-methoxy-4-oxo-1,4-dihydro-quinoline-3-carboxylic acid (Compound #15)

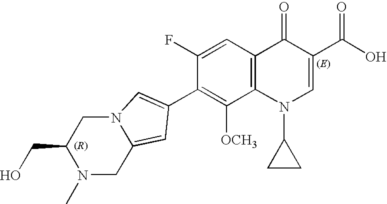

### Step A: 7-(3R-tert-Butoxymethyl-2-methyl-1,2,3,4-tetrahydro-pyrrolo[1,2-a]pyrazin-7-yl)-1-cyclopropyl-6-fluoro-8-methoxy-4-oxo-1,4-dihydro-quinoline-3-carboxylic acid ethyl ester

The product was prepared in a manner analogous to the process outlined in Step B, Example 13, reacting Intermediate Compound 11 in place of Intermediate Compound 8.

MS 526 (M+H).

### Step B: 1-Cyclopropyl-6-fluoro-7-(3R-hydroxymethyl-2-methyl-1,2,3,4-tetrahydro-pyrrolo[1,2-a]pyrazin-7-yl)-8-methoxy-4-oxo-1,4-dihydro-quinoline-3-carboxylic acid

To a solution of the product from Step A above (40 mg, 0.076 mmol) in MeOH (1 mL) and THF (1 mL) was added aq. 1N NaOH (1 mL). The resulting mixture was heated at 55° C. for 1.5 hours, then cooled to room temperature and acidified with aq. 1N HCl to pH 5. The resulting mixture was extracted with EtOAc. The organic solution was washed with brine, dried over NaSO₄, and concentrated. The resulting residue was stirred in TFA (1 mL) at room temperature for 6 hours, then concentrated. The resulting residue was dissolved in a small amount of CH₂Cl₂ and diluted with diethyl ether. The resulting solid was collected by filtration and dried in vacuo to yield the title compound.

MS 442 (M+H).

### EXAMPLE 16

### 1-Cyclopropyl-6-fluoro-8-methoxy-7-(1S-methyl-1,2,3,4-tetrahydro-pyrrolo[1,2-a]pyrazin-7-yl)-4-oxo-1,4-dihydro-quinoline-3-carboxylic acid (Compound #16)

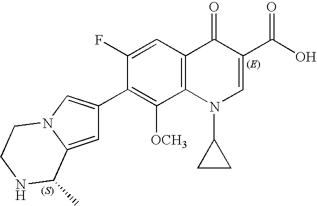

Compound 16 was prepared in a manner analogous to Compound 1, reacting Intermediate Compound 12 in place of Intermediate Compound 7 and using ethyl 1-cyclopropyl-1,4-dihydro-6-fluoro-8-methoxy-4-oxo-7-[(trifluoromethyl-sulfonyl)oxy]-quinoline-3-carboxylate (Intermediate Compound 2) in place of ethyl 7-bromo-1-cyclopropyl-8-difluoromethoxy-1,4-dihydro-4-oxo-quinoline-3-carboxylate.

MS 412 (M+H).

### EXAMPLE 17

### 1-Cyclopropyl-6-fluoro-8-methoxy-7-(1R-methyl-1,2,3,4-tetrahydro-pyrrolo[1,2-a]pyrazin-7-yl)-4-oxo-1,4-dihydro-quinoline-3-carboxylic acid (Compound #17)

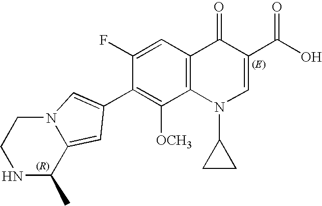

Compound 17 was prepared in a manner analogous to Compound 1, reacting Intermediate Compound 13 in place of Intermediate Compound 7 and using ethyl 1-cyclopropyl-1,4-dihydro-6-fluoro-8-methoxy-4-oxo-7-[(trifluoromethyl-sulfonyl)oxy]-quinoline-3-carboxylate (Intermediate Compound 2) in place of ethyl 7-bromo-1-cyclopropyl-8-difluoromethoxy-1,4-dihydro-4-oxo-quinoline-3-carboxylate.

MS 412 (M+H).

### EXAMPLE 18

### 1-Cyclopropyl-7-(1R,3S-dimethyl-1,2,3,4-tetrahydro-pyrrolo[1,2-a]pyrazin-7-yl)-6-fluoro-8-methoxy-4-oxo-1,4-dihydro-quinoline-3-carboxylic acid (Compound #18)

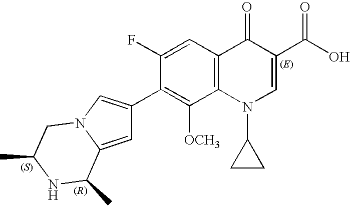

Compound 18 was prepared in a manner analogous to Compound 1, reacting with Intermediate Compound 14 in place of Intermediate Compound 7 and ethyl 1-cyclopropyl-1,4-dihydro-6-fluoro-8-methoxy-4-oxo-7-[(trifluoromethyl-sulfonyl)oxy]-quinoline-3-carboxylate (Intermediate Compound 2) in place of ethyl 7-bromo-1-cyclopropyl-8-difluoromethoxy-1,4-dihydro-4-oxo-quinoline-3-carboxylate.

MS 426 (M+H).

### EXAMPLE 19

### 1-Cyclopropyl-6-fluoro-7-(3S-methyl-1,2,3,4-tetrahydro-pyrrolo[1,2-a]pyrazin-7-yl)-4-oxo-1,4-dihydro-quinoline-3-carboxylic acid (Compound #19)

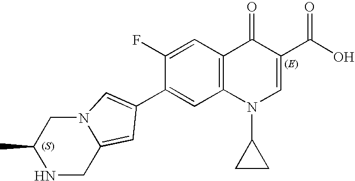

Compound 19 was prepared in a manner analogous to Compound 1, reacting Intermediate Compound 5 in place of Intermediate Compound 7 and using ethyl 1-cyclopropyl-1,4-dihydro-6-fluoro-4-oxo-7-[(trifluoromethyl-sulfonyl)oxy]-quinoline-3-carboxylate (Intermediate Compound 1) in place of ethyl 7-bromo-1-cyclopropyl-8-difluoromethoxy-1,4-dihydro-4-oxo-quinoline-3-carboxylate.

MS 382 (M+H).

### EXAMPLE 20

### 1-Cyclopropyl-7-(2,3S-dimethyl-1,2,3,4-tetrahydro-pyrrolo[1,2-a]pyrazin-7-yl)-6-fluoro-4-oxo-1,4-dihydro-quinoline-3-carboxylic acid (Compound #20)

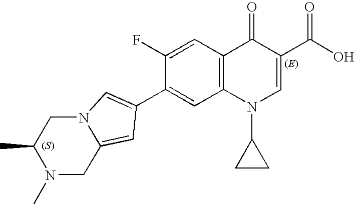

Compound 20 was prepared in a manner analogous to Compound 13, reacting ethyl 1-cyclopropyl-1,4-dihydro-6-fluoro-4-oxo-7-[(trifluoromethyl-sulfonyl)oxy]-quinoline-3-carboxylate (Intermediate Compound 1) in place of ethyl 1-cyclopropyl-1,4-dihydro-6-fluoro-8-methoxy-4-oxo-7-[(trifluoromethyl-sulfonyl)oxy]-quinoline-3-carboxylate (Intermediate Compound 2).

MS 396 (M+H).

### EXAMPLE 21

### 1-Cyclopropyl-6-fluoro-7-(1S-methyl-1,2,3,4-tetrahydro-pyrrolo[1,2-a]pyrazin-7-yl)-4-oxo-1,4-dihydro-quinoline-3-carboxylic acid (Compound #21)

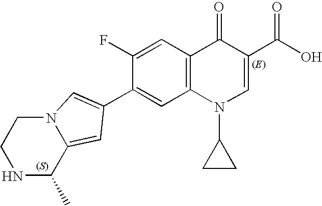

Compound 21 was prepared in a manner analogous to Compound 1, reacting Intermediate Compound 12 in place of Intermediate Compound 7 and using ethyl 1-cyclopropyl-1,4-dihydro-6-fluoro-4-oxo-7-[(trifluoromethyl-sulfonyl)oxy]-quinoline-3-carboxylate (Intermediate Compound 1) in place of ethyl 1-cyclopropyl-1,4-dihydro-6-fluoro-8-methoxy-4-oxo-7-[(trifluoromethyl-sulfonyl)oxy]-quinoline-3-carboxylate (Intermediate Compound 2).

MS 382 (M+H).

### EXAMPLE 22

### 1-Cyclopropyl-6-fluoro-7-(1R-methyl-1,2,3,4-tetrahydro-pyrrolo[1,2-a]pyrazin-7-yl)-4-oxo-1,4-dihydro-quinoline-3-carboxylic acid (Compound #22)

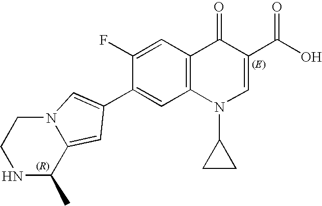

Compound 22 was prepared in a manner analogous to Compound 1, reacting Intermediate Compound 13 in place of Intermediate Compound 7 and using ethyl 1-cyclopropyl-1,4-dihydro-6-fluoro-4-oxo-7-[(trifluoromethyl-sulfonyl)oxy]-quinoline-3-carboxylate (Intermediate Compound 1) in place of ethyl 1-cyclopropyl-1,4-dihydro-6-fluoro-8-methoxy-4-oxo-7-[(trifluoromethyl-sulfonyl)oxy]-quinoline-3-carboxylate (Intermediate Compound 2).

MS 382 (M+H).

### EXAMPLE 23

### 8-Fluoro-3S-methyl-9-(3S-methyl-1,2,3,4-tetrahydro-pyrrolo[1,2-a]pyrazin-7-yl)-6-oxo-2,3-dihydro-6H-1-oxa-3a-aza-phenalene-5-carboxylic acid (Compound #23)

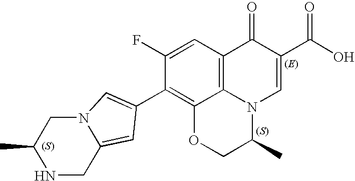

Compound 23 was prepared in a manner analogous to Compound 1, reacting Intermediate Compound 5 in place of Intermediate Compound 7 and using ethyl 2,3-dihydro-9-fluoro-3-methyl-7-oxo-7H-pyrido[1,2,3-de]-10-[(trifluoromethylsulfonyl)oxy]-1,4-benzoxazine-6-carboxylate (Intermediate Compound 4) in place of ethyl 7-bromo-1-cyclopropyl-8-difluoromethoxy-1,4-dihydro-4-oxo-quinoline-3-carboxylate.

MS 398 (M+H).

### EXAMPLE 24

### 8-Fluoro-3S-methyl-9-(1S-methyl-1,2,3,4-tetrahydro-pyrrolo[1,2-a]pyrazin-7-yl)-6-oxo-2,3-dihydro-6H-1-oxa-3a-aza-phenalene-5-carboxylic acid (Compound #24)

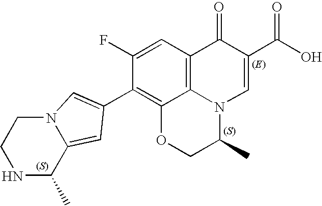

Compound 24 was prepared in a manner analogous to Compound 1, reacting Intermediate Compound 12 in place of Intermediate Compound 7 and using ethyl 2,3-dihydro-9-fluoro-3-methyl-7-oxo-7H-pyrido[1,2,3-de]-10-[(trifluoromethylsulfonyl)oxy]-1,4-benzoxazine-6-carboxylate (Intermediate Compound 4) in place of ethyl 7-bromo-1-cyclopropyl-8-difluoromethoxy-1,4-dihydro-4-oxo-quinoline-3-carboxylate.

MS 398 (M+H).

### EXAMPLE 25

### 1-Cyclopropyl-6-fluoro-7-(3S-methyl-1,2,3,4-tetrahydro-pyrrolo[1,2-a]pyrazin-7-yl)-4-oxo-1,4-dihydro-[1,8]naphthyridine-3-carboxylic acid (Compound #25)

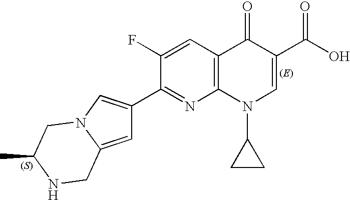

Compound 25 was prepared in a manner analogous to Compound 1, reacting Intermediate Compound 5 in place of Intermediate Compound 7 and using ethyl 7-chloro-1-cyclopropyl-6-fluoro-4-oxo-1,4-dihydro-naphthpyridine-3-carboxylate in place of ethyl 7-bromo-1-cyclopropyl-8-difluoromethoxy-1,4-dihydro-4-oxo-quinoline-3-carboxylate.

MS 383 (M+H).

### EXAMPLE 26

### 1-Cyclopropyl-6-fluoro-7-(1S-methyl-1,2,3,4-tetrahydro-pyrrolo[1,2-a]pyrazin-7-yl)-4-oxo-1,4-dihydro-[1,8]naphthyridine-3-carboxylic acid (Compound #26)

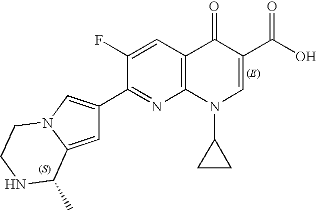

Compound 26 was prepared in a manner analogous to Compound 1, reacting Intermediate Compound 12 in place of Intermediate Compound 7 and using ethyl 7-chloro-1-cyclopropyl-6-fluoro-4-oxo-1,4-dihydro-naphthpyridine-3-carboxylate in place of ethyl 7-bromo-1-cyclopropyl-8-difluoromethoxy-1,4-dihydro-4-oxo-quinoline-3-carboxylate.

MS 383 (M+H).

### EXAMPLE 27

### 1-(2,4-Difluoro-phenyl)-6-fluoro-7-(3S-methyl-1,2,3,4-tetrahydro-pyrrolo[1,2-a]pyrazin-7-yl)-4-oxo-1,4-dihydro-[1,8]naphthyridine-3-carboxylic acid (Compound #27)

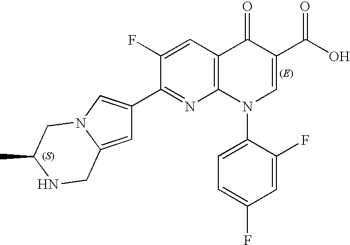

Compound 27 was prepared in a manner analogous to Compound 1, reacting Intermediate Compound 5 in place of Intermediate Compound 7 and using ethyl 7-chloro-1-(2,4-difluorophenyl)-6-fluoro-4-oxo-1,4-dihydro-naphthyridine-3-carboxylate in place of ethyl 7-bromo-1-cyclopropyl-8-difluoromethoxy-1,4-dihydro-4-oxo-quinoline-3-carboxylate.

MS 455 (M+H).

### EXAMPLE 28

### 1-(2,4-Difluoro-phenyl)-6-fluoro-7-(1S-methyl-1,2,3,4-tetrahydro-pyrrolo[1,2-a]pyrazin-7-yl)-4-oxo-1,4-dihydro-[1,8]naphthyridine-3-carboxylic acid (Compound #28)

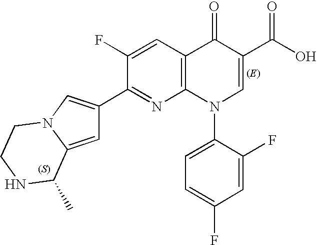

Compound 28 was prepared in a manner analogous to Compound 1, reacting Intermediate Compound 12 in place of Intermediate Compound 7 and using ethyl 7-chloro-1-(2,4-difluorophenyl)-6-fluoro-4-oxo-1,4-dihydro-naphthyridine-3-carboxylate in place of ethyl 7-bromo-1-cyclopropyl-8-difluoromethoxy-1,4-dihydro-4-oxo-quinoline-3-carboxylate.

MS 455 (M+H).

### EXAMPLE 29

### 1-Cyclopropyl-8-difluoromethoxy-6-fluoro-7-(1S-methyl-1,2,3,4-tetrahydro-pyrrolo[1,2-a]pyrazin-7-yl)-4-oxo-1,4-dihydro-quinoline-3-carboxylic acid (Compound #29)

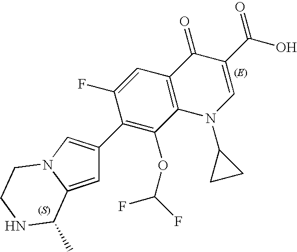

Compound 29 was prepared in a manner analogous to Compound 1, reacting Intermediate Compound 12 in place of Intermediate Compound 7 and ethyl 7-bromo-1-cyclopropyl-8-difluoromethoxy-6-fluoro-1,4-dihydro-4-oxo-quinoline-3-carboxylic acid (Intermediate Compound 3) in place of ethyl 7-bromo-1-cyclopropyl-8-difluoromethoxy-1,4-dihydro-4-oxo-quinoline-3-carboxylate.

MS 448 (M+H).

### EXAMPLE 30

### 7-(2-Benzyl-1,2,3,4-tetrahydro-pyrrolo[1,2-a]pyrazin-7-yl)-1-(2,4-difluoro-phenyl)-6-fluoro-4-oxo-1,4-dihydro-[1,8]naphthyridine-3-carboxylic acid (Compound #30)

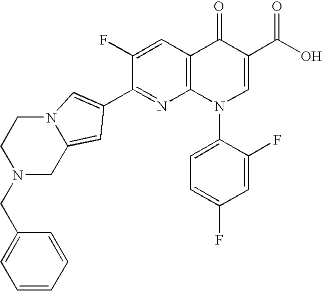

### Step A: 7-(2-Benzyl-1,2,3,4,6,8a-hexahydro-pyrrolo[1,2S-a]pyrazin-7-yl)-1-(2,4-difluoro-phenyl)-6-fluoro-4-oxo-1,4-dihydro-[1,8]naphthyridine-3-carboxylic acid ethyl ester

7-(2-Benzyl-1,2,3,4,6,8a-hexahydro-pyrrolo[1,2S-a]pyrazin-7-yl)-1-(2,4-difluoro-phenyl)-6-fluoro-4-oxo-1,4-dihydro-[1,8]naphthyridine-3-carboxylic acid ethyl ester was prepared as described in Example 2, Step A, using Intermediate Compound 10 and ethyl 7-chloro-1-(2,4-difluorophenyl)-6-fluoro-4-oxo-1,4-dihydronaphthyridine-3-carboxylate.

MS 505 (M+H).

### Step B: 7-(2-Benzyl-1,2,3,4-tetrahydro-pyrrolo[1,2-a]pyrazin-7-yl)-1-(2,4-difluoro-phenyl)-6-fluoro-4-oxo-1,4-dihydro-[1,8]naphthyridine-3-carboxylic acid ethyl ester

7-(2-Benzyl-1,2,3,4-tetrahydro-pyrrolo[1,2-a]pyrazin-7-yl)-1-(2,4-difluoro-phenyl)-6-fluoro-4-oxo-1,4-dihydro-[1,8]naphthyridine-3-carboxylic acid ethyl ester was prepared from 7-(2-benzyl-1,2,3,4,6,8a-hexahydro-pyrrolo[1,2-a]pyrazin-7-yl)-1-(2,4-difluoro-phenyl)-6-fluoro-4-oxo-1,4-dihydro-[1,8]naphthyridine-3-carboxylic acid ethyl ester as described in Example 1, Step B.

MS 503 (M+H).

### Step C: 7-(2-Benzyl-1,2,3,4-tetrahydro-pyrrolo[1,2-a]Pyrazin-7-yl)-1-(2,4-difluoro-phenyl)-6-fluoro-4-oxo-1,4-dihydro-[1,8]naphthyridine-3-carboxylic acid

7-(2-Benzyl-1,2,3,4-tetrahydro-pyrrolo[1,2-a]pyrazin-7-yl)-1-(2,4-difluoro-phenyl)-6-fluoro-4-oxo-1,4-dihydro-[1,8]naphthyridine-3-carboxylic acid was prepared from 7-(2-Benzyl-1,2,3,4-tetrahydro-pyrrolo[1,2-a]pyrazin-7-yl)-1-(2,4-difluoro-phenyl)-6-fluoro-4-oxo-1,4-dihydro-[1,8]naphthyridine-3-carboxylic acid ethyl ester as described in Example 2, Step B.

MS 531 (M+H).

### EXAMPLE 31

### 7-(2-Benzyl-1,2,3,4-tetrahydro-pyrrolo[1,2-a]pyrazin-7-yl)-1-cyclopropyl-6-fluoro-8-methoxy-4-oxo-1,4-dihydro-quinoline-3-carboxylic acid (Compound #31)

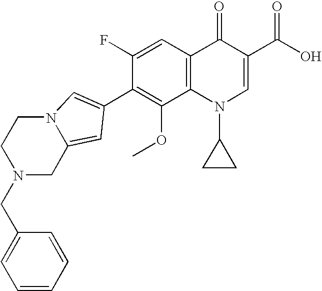

Compound 31 was prepared according to the process described in Example 30, reacting Intermediate Compound 2 and Intermediate Compound 10.

MS 488 (M+H).

### EXAMPLE 32

### 1-Cyclopropyl-6-fluoro-7-(1,2,3,4,6,8a-hexahydro-pyrrolo[1,2S-a]pyrazin-7-yl)-8-methoxy-4-oxo-1,4-dihydro-quinoline-3-carboxylic acid (Compound #32)

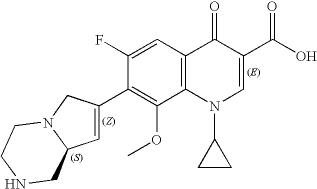

### Step A: 7-(2-tert-Butoxycarbonyl-1,2,3,4,6,8a-hexahydro-pyrrolo[1,2S-a]pyrazin-7-yl)-1-cyclopropyl-6-fluoro-8-methoxy-4-oxo-1,4-dihydro-quinoline-3-carboxylic acid ethyl ester

7-(2-tert-Butoxycarbonyl-1,2,3,4,6,8a-hexahydro-pyrrolo[1,2S-a]pyrazin-7-yl)-1-cyclopropyl-6-fluoro-8-methoxy-4-oxo-1,4-dihydro-quinoline-3-carboxylic acid ethyl ester was prepared from Intermediate Compounds 2 and 7 in a manner analogous to Example 1, Step A.

MS 528 (M+H).

### Step B: 1-Cyclopropyl-6-fluoro-7-(1,2,3,4,6,8a-hexahydro-pyrrolo[1,2-a]pyrazin-7-yl)-8-methoxy-4-oxo-1,4-dihydro-quinoline-3-carboxylic acid.

The title compound was prepared from 7-(2-tert-Butoxycarbonyl-1,2,3,4,6,8a-hexahydro-pyrrolo[1,2S-a]pyrazin-7-yl)-1-cyclopropyl-6-fluoro-8-methoxy-4-oxo-1,4-dihydro-quinoline-3-carboxylic acid ethyl ester in a manner analogous to Example 1, Step C.

MS 400 (M+H).

### EXAMPLE 33

### 8-Fluoro-9-(1,2,3,4,6,8a-hexahydro-pyrrolo[12-a]pyrazin-7-yl)-3S-methyl-6-oxo-2,3-dihydro-6H-1-oxa-3a-aza-phenalene-5-carboxylic acid (Compound #33)

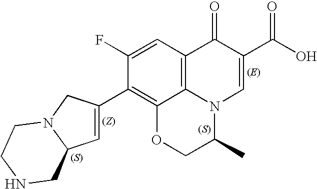

Compound 33 was prepared in a manner analogous to that of Compound 32 reacting Intermediate Compound 4 and Intermediate Compound 7.

### EXAMPLE 34

### 1-Cyclopropyl-6-fluoro-8-methoxy-4-oxo-7-(1,2,3,4-tetrahydro-pyrrolo[1,2-a]pyrazin-7-yl)-1,4-dihydro-quinoline-3-carboxylic acid (Compound #34)

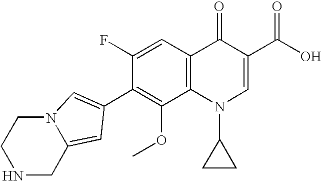

Compound 34 was prepared in a manner analogous to that of Compound 1 reacting Intermediate Compound 2 and Intermediate Compound 7.

MS 398 (M+H).

### EXAMPLE 35

### 8-Fluoro-3S-methyl-6-oxo-9-(1,2,3,4-tetrahydro-pyrrolo[1,2-a]pyrazin-7-yl)-2,3-dihydro-6H-1-oxa-3a-aza-phenalene-5-carboxylic acid (Compound #35)

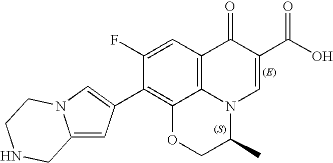

Compound 35 was prepared in a manner analogous to that of Compound 1 reacting from Intermediate Compound 4 and Intermediate Compound 7.

MS 384 (M+H).

### EXAMPLE 36

### Biological Activity

The compounds of the present invention possess antibacterial activity and are therefore useful as antibacterial agents for the treatment of bacterial infections in humans and animals.

Minimal inhibitory concentration (MIC) is an indicator of in vitro antibacterial activity. The MIC is the lowest concentration of test compound that completely inhibits growth of the test organism. The in vitro antimicrobial activity of representative compounds of the present invention was determined by the microdilution broth method following the test method from the National Committee for Clinical Laboratory Standards (NCCLS). This method is described in the NCCLS Document M7-A4, Vol. 17, No. 2, “Methods for Dilution Antimicrobial Susceptibility Test for Bacteria that Grow Aerobically—Fourth Edition”, which is incorporated herein by reference.

In this method two-fold serial dilutions of drug in cation adjusted Mueller-Hinton broth were added to wells in microdilution trays. The test organisms were prepared by adjusting the turbidity of actively growing broth cultures so that the final concentration of test organism after it is added to the wells was approximately 5×10⁴ CFU/well. Following inoculation of the microdilution trays, the trays were incubated at 35° C. for 16-20 hours and then read. The amount of growth in the wells containing the test compound was compared with the amount of growth in the growth-control wells (no test compound) used in each tray.

Representative compounds of the present invention were tested against a variety of pathogenic bacteria with results as listed in Table 3, below.

TABLE 3
In vitro Antibacterial Activity (MIC in μg/mL)

| | MIC (μg/mL) | | | | |
|---|---|---|---|---|---|
| ID NO. | A | B | C | D | E |
| 1 | 0.06 | 1 | 8 | 8 | 0.5 |
| 2 | 0.5 | >16 | >16 | >16 | 8 |
| 3 | 8.0 | >16 | >16 | >16 | >16 |
| 4 | 0.25 | 2 | >16 | >16 | 2 |
| 5 | 8.0 | >16 | >16 | >16 | >16 |
| 6 | 4.0 | >16 | >16 | >16 | >16 |
| 7 | 0.12 | 1 | 8 | 16 | 1 |
| 8 | 0.5 | 4 | 16 | >16 | 1 |
| 9 | 0.5 | 4 | 16 | >16 | 1 |
| 10 | 0.25 | 4 | 16 | >16 | 2 |
| 11 | 0.03 | 0.5 | 1 | 4 | 0.12 |
| 12 | 0.03 | 1 | 2 | 8 | 0.25 |
| 13 | 0.12 | 0.5 | 4 | 8 | 0.5 |
| 14 | 0.25 | 2 | 8 | >16 | 1 |
| 15 | 0.12 | 4 | 8 | 16 | 1 |
| 16 | 0.12 | 2 | 4 | 8 | 0.5 |
| 17 | 0.25 | 8 | 16 | >16 | 2 |
| 18 | 0.12 | 2 | 8 | 16 | 1 |
| 19 | 0.06 | 2 | 4 | >16 | 0.5 |
| 20 | 0.5 | 4 | 16 | >16 | 1 |
| 21 | 0.25 | 4 | 16 | 16 | 1 |
| 22 | 0.25 | 4 | 16 | >16 | 1 |
| 23 | 0.25 | 4 | 8 | 16 | 1 |
| 24 | 0.25 | 4 | 8 | 16 | 1 |
| 25 | 0.06 | 2 | 8 | 8 | 0.5 |
| 26 | 0.5 | 8 | 8 | 16 | 1 |
| 27 | 0.12 | 16 | 16 | >16 | 1 |
| 28 | 1.0 | 16 | >16 | >16 | 4 |
| 29 | 0.25 | 1 | 8 | 16 | 2 |
| 30 | 2.0 | 8 | 8 | 16 | 8 |
| 31 | 0.25 | 8 | >16 | >16 | 2 |
| 32 | 0.25 | 16 | >16 | 16 | 2 |
| 33 | 0.25 | >16 | >16 | >16 | 2 |
| 34 | 0.03 | 1 | 1 | 1 | 0.25 |
| 35 | 0.5 | >16 | >16 | >16 | 2 |

A: Staphylococcus aureus OC4172; strains B, C, and D are fluoroquinolone-resistant clinical isolates of Streptococcus pneumoniae that contain different constellations of amino acid substitutions in the QRDR region;
E: Streptococcus pneumoniae ATCC 49619.
The abbreviation “ND” indicates that the value was not determined.

### EXAMPLE 37

### Oral Formulation—Prophetic Example

As a specific embodiment of an oral composition, 100 mg of the compound prepared as in Example 11 or Example 16 is formulated with sufficient finely divided lactose to provide a total amount of 580 to 590 mg to fill a size O hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed:

1. A compound of formula (I) 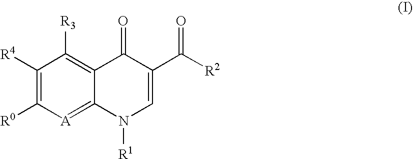
wherein
A is selected from the group consisting of N and CR²⁰; wherein R²⁰ is selected from the group consisting of hydrogen, fluoro, chloro, hydroxy, C₁₋₄alkyl, halogenated C₁₋₄alkyl, C₁₋₄alkoxy, halogenated C₁₋₄alkoxy, C₁-C₄alkylthio, amino, (C₁₋₄alkyl)amino, di(C₁₋₄alkyl)amino and cyano;
alternatively, A is CR²⁰, and R¹ and R²⁰ are taken together with the atoms to which they are bound to form 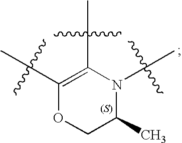
R¹ is selected from the group consisting of C₃-C₆cycloalkyl, C₄-C₆heterocycloalkyl, C₁-C₈alkyl, C₂-C₈alkenyl, phenyl and a 5 to 6 membered heteroaryl; wherein the C₃-C₆cycloalkyl, C₄-C₆heterocycloalkyl, phenyl or 5 to 6 membered heteroaryl is optionally substituted with one or more substituents independently selected from the group consisting of fluoro, chloro, C₁₋₄alkyl, C₁₋₄alkoxy, cyano, nitro, amino, (C₁₋₄alkyl)amino and di(C₁₋₄alkyl)amino;
R² is selected from the group consisting of hydroxy, C₁₋₄alkoxy and benzyloxy;
R³ and R⁴ are each independently selected from the group consisting of hydrogen, fluoro, chloro, hydroxy, amino, (C₁₋₄alkyl)amino, di(C₁₋₄alkyl)amino, C₁₋₄alkyl, halogenated C₁₋₄alkyl, C₁₋₄alkoxy, halogenated C₁₋₄alkoxy and C₁-C₄alkylthio;
R⁰ is selected from the group consisting of 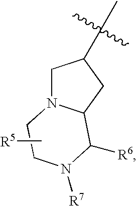 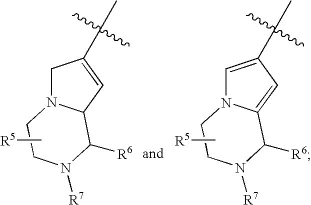
R⁵ and R⁶ are each independently selected from the group consisting of hydrogen, C₁₋₄alkyl, hydroxy substituted C₁₋₄alkyl, halogenated C₁₋₄alkyl, oxo, C₃₋₈cycloalkyl and phenyl;
provided that when R⁶ is oxo and R⁵is oxo, then R⁵ is bound to the carbon atom that is alpha to the nitrogen of the ring fusion;
R⁷is selected from the group consisting hydrogen, C₁₋₄alkyl, C₃₋₈cycloalkyl, —(C₁₋₄alkyl)-C₃₋₈cycloalkyl, aryl, aralkyl, heteroaryl, —(C₁₋₄alkyl)-heteroaryl, heterocycloalkyl, —(C₁₋₄alkyl)-heterocycloalkyl, —C(O)—R , —C(O)O—R⁸, —C(O)—NR⁹R¹⁰ and —C(O)—(C₁₋₄alkyl)-NR⁹R¹⁰;
wherein the C₁-₄alkyl is optionally substituted with one or more substituents independently selected from the group consisting of halogen, hydroxy, oxo, thio, cyano, —NR¹¹R¹², aryloxy, heteroaryloxy, acyloxy, carboxy, carboxamido and acylamino;
wherein the C₃₋₈cycloalkyl, whether alone or as part of a substituent group is optionally substituted with one or more fluoro;
and wherein the aryl, heteroaryl or heterocycloalkyl, whether alone or as part of a substituent group, is optionally substituted with one to three substituents independently selected from the group consisting of halogen, hydroxy, oxo, cyano, thio, nitro, —NR¹³R¹⁴, C₁-C₈alkyl, halogenated C₁-C₈alkyl, C₁-C₈alkoxy, halogenated C₁-C₈alkoxy, C₁-C₈alkylthio, formyl, carboxy, —C(O)O—(C₁₋₄alkyl), —O—C(O)—(C₁-C₄alkyl), —NR¹⁵—C(O)—(C₁-C₄alkyl) and —C(O)—NR¹⁶R¹⁷; R⁸ is selected from the group consisting of C₁₋₄alkyl;
R⁹ and R¹⁰ are each independently selected from the group consisting of hydrogen and C₁₋₄alkyl; alternatively, R⁹ and R¹⁰ are taken together with the nitrogen atom to which they are bound to form a 5 to 6 membered nitrogen containing saturated ring structure;
R¹¹ and R¹² are each independently selected from the group consisting of hydrogen and C₁₋₄alkyl; alternatively, R¹¹ and R¹² are taken together with the nitrogen atom to which they are bound to form a 5 to 6 membered nitrogen containing saturated ring structure;
R¹³ and R¹⁴ are each independently selected from the group consisting of hydrogen and C₁₋₄alkyl; alternatively, R¹³ and R¹⁴ are taken together with the nitrogen atom to which they are bound to form a 5 to 6 membered nitrogen containing saturated ring structure;
R¹⁵ is selected from the group consisting of C₁₋₄alkyl;
R¹⁶ and R¹⁷ are each independently selected from the group consisting of hydrogen and C₁₋₄alkyl; alternatively, R¹⁶ and R¹⁷ are taken together with the nitrogen atom to which they are bound to form a 5 to 6 membered nitrogen containing saturated ring structure;
or optical isomer, diastereomer, enantiomer, pharmaceutically acceptable salt, hydrate, or prodrug thereof.

2. A compound as in claim 1, wherein:
A is selected from the group N and CR²⁰; wherein R₂₀ is selected from the group consisting of hydrogen, fluoro, chloro, hydroxy, _{C1-4}alkyl, fluorinated C₁₋₄alkyl, C₁₋₄alkoxy and fluorinated C₁₋₂alkoxy;
alternatively, A is CR²⁰, and R²⁰ and R¹ are taken together with the atoms to which they are bound to form 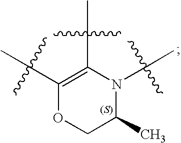
R¹ is selected from the group consisting of C₃-C₆cycloalkyl, C₄-C₆heterocycloalkyl, phenyl and a 5 to 6 membered heteroaryl; wherein the C₃-C₆cycloalkyl, C₄-C₆heterocycloalkyl, phenyl or 5 to 6 membered heteroaryl is optionally substituted with one to two substituents independently selected from the group consisting of fluoro, chloro, C₁₋₄alkyl, C₁₋₄alkoxy, cyano, nitro, amino, (C₁₋₄alkyl)amino and di(C₁₋₄alkyl)amino;
R² is selected from the group consisting of hydroxy, C₁₋₄alkoxy and benzyloxy;
R³ and R⁴ are each independently selected from the group consisting of hydrogen, fluoro, chloro, hydroxy, fluorinated C₁₋₄alkyl, C₁₋₄alkoxy and fluorinated C₁₋₄alkoxy;
R⁰ is selected from the group consisting of 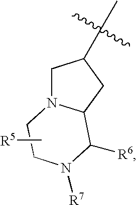 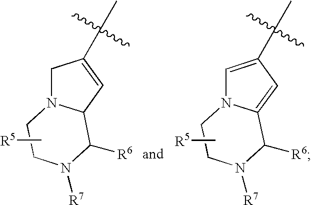
R⁵ and R⁶ are each independently selected from the group consisting of hydrogen, C₁₋₄alkyl, hydroxy substituted C₁₋₄alkyl, halogenated C₁₋₄alkyl and oxo; provided that when R⁶ is oxo and R⁵ is oxo, then R⁵ is bound to the carbon atom which is alpha to the nitrogen of the ring fusion;
R⁷ is selected from the group consisting hydrogen, C₁₋₄alkyl, —(C₁₋₄alkyl)-C₃₋₈cycloalkyl, aralkyl, —(C₁₋₄alkyl)-heteroaryl, —(C₁₋₄alkyl)-heterocycloalkyl, —C(O)—R⁸ and —C(O)—(C₁₋₄alkyl)-NR⁹R¹⁰;
wherein R⁸ is selected from the group consisting of C₁₋₄alkyl;
and wherein R⁹ and R¹⁰ are each independently selected from the group consisting of hydrogen and C₁₋₄alkyl; alternatively, R⁹ and R¹⁰ are taken together with the nitrogen atom to which they are bound to form a 5 to 6 membered nitrogen containing saturated ring structure;
or optical isomer, diastereomer, enantiomer, pharmaceutically acceptable salt, hydrate, or prodrug thereof.

3. A compound as in claim 2, wherein:
A is selected from the group N and CR²⁰; wherein R²⁰ is selected from the group consisting of hydrogen, C₁₋₂alkoxy and fluorinated C₁₋₂alkoxy; alternatively, A is CR²⁰, and R²⁰ and R¹ are taken together with the atoms to which they are bound to form 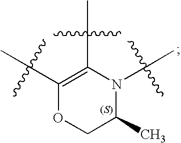
R¹ is selected from the group consisting of C₃₋₆cycloalkyl and phenyl; wherein the phenyl is optionally substituted with one to two halogen;
R² is hydroxy;
R³ and R⁴ are each independently selected from the group consisting of hydrogen and fluoro;
R⁰ is selected from the group consisting of 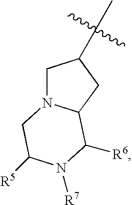 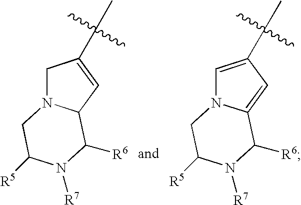
R⁵ and R⁶ are each independently selected from the group consisting of hydrogen, C₁₋₄alkyl and hydroxy substituted C₁₋₄alkyl;
R⁷ is selected from the group consisting of hydrogen, C₁₋₄alkyl, aralkyl and —C(O)—(C₁₋₄alkyl)-NR⁹R¹⁰; wherein R⁹ and R¹⁰ are each independently selected form the group consisting of hydrogen and C₁₋₄alkyl;
or optical isomer, diastereomer, enantiomer, pharmaceutically acceptable salt, hydrate, or prodrug thereof.

4. A compound as in claim 3, wherein:
A is selected from the group N, CH, C—OCH₃ and C—OCHF₂;
alternatively, A is CR²⁰, and R²⁰ and R¹ are taken together with the atoms to which they are bound to form 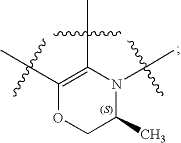
R¹ is selected from the group consisting of cyclopropyl and 2,4-difluorophenyl;
R² is hydroxy;
R³ is hydrogen;
R⁴ is selected from the group consisting of hydrogen and fluoro;
R⁰ is selected from the group consisting of 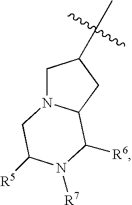 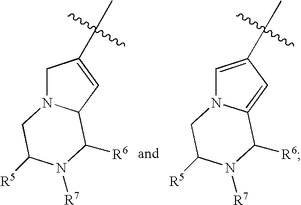
R⁵ and R⁶ are each independently selected from the group consisting of hydrogen, methyl and hydroxymethyl;
R⁷ is selected from the group consisting of hydrogen, methyl, benzyl and 1-(2S-methylaminopropionyl)-;
or optical isomer, diastereomer, enantiomer, pharmaceutically acceptable salt, hydrate, or prodrug thereof.

5. A compound as in claim 1, wherein R⁰ is 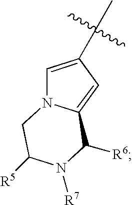
or optical isomer, diastereomer, enantiomer, pharmaceutically acceptable salt, hydrate, or prodrug thereof.

6. A compound as in claim 1, wherein R⁰ is 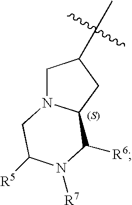
or optical isomer, diastereomer, enantiomer, pharmaceutically acceptable salt, hydrate, or prodrug thereof.

7. A compound as in claim 1, wherein R⁰ is 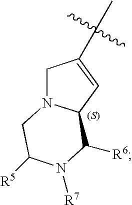
or optical isomer, diastereomer, enantiomer, pharmaceutically acceptable salt, hydrate, or prodrug thereof.

8. A compound as in claim 1, wherein A is CR²⁰ and wherein R¹ and R²⁰ are taken together with the atoms to which they are bound to form 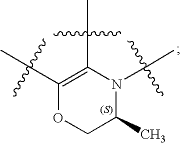
or optical isomer, diastereomer, enantiomer, pharmaceutically acceptable salt, hydrate, or prodrug thereof.

9. A compound as in claim 4, wherein:
A is selected from the group N, CH, C—OCH₃ and C—OCHF₂;
alternatively, A is CR²⁰, and R²⁰ and R¹ are taken together with the atoms to which they are bound to form 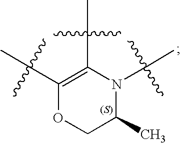
R¹ is selected from the group consisting of cyclopropyl and 2,4-difluorophenyl;
R² is hydroxy;
R³ is hydrogen;
R⁴ is selected from the group consisting of hydrogen and fluoro;
R⁰ is 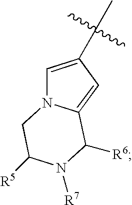
R⁵ is selected from the group consisting of hydrogen, methyl and hydroxymethyl; and wherein R⁵ is bound to the piperazinyl portion of R⁰ at the carbon atom that is alpha to the N—R⁷ portion of R⁰;
R⁶ is selected from the group consisting of hydrogen and methyl;
R⁷ is selected from the group consisting of hydrogen, methyl and 1-(2S-methylaminopropionyl)-;
or optical isomer, diastereomer, enantiomer, pharmaceutically acceptable salt, hydrate, or prodrug thereof.

10. A compound as in claim 4, selected from the group consisting of:
1-Cyclopropyl-8-difluoromethoxy-4-oxo-7-(1,2,3,4-tetrahydro-pyrrolo[1,2-a]pyrazin-7-yl)-1,4-dihydro-quinoline-3-carboxylic acid;
7-(2-Benzyl-1,2,3,4,6,8a-hexahydro-pyrrolo[1,2-a]pyrazin-7-yl)-1-cyclopropyl-8-difluoromethoxy-4-oxo-1,4-dihydro-quinoline-3-carboxylic acid;
1-Cyclopropyl -8-difluoromethoxy-7-(octahydro-pyrrolo[1,2-a]pyrazin-7-yl)-4-oxo-1,4-dihydro-quinoline-3-carboxylic acid;
1-Cyclopropyl-8-difluoromethoxy-7-(2-methyl-1,2,3,4-tetrahydro-pyrrolo[1,2-a]pyrazin-7-yl)-4-oxo-1,4-dihydro-quinoline-3-carboxylic acid;
1-Cyclopropyl-8-difluoromethoxy-7-(2-methyl-octahydro-pyrrolo[1,2-a]pyrazin-7-yl)-4-oxo-1,4-dihydro-quinoline-3-carboxylic acid;
1-Cyclopropyl-8-difluoromethoxy-7-(3S-methyl-octahydro-pyrrolo[1,2-a]pyrazin-7-yl)-4-oxo-1,4-dihydro-quinoline-3-carboxylic acid;
1-Cyclopropyl-8-difluoromethoxy-7-(3S-methyl-1,2,3,4-tetrahydro-pyrrolo[1,2-a]pyrazin-7-yl)-4-oxo-1,4-dihydro-quinoline-3-carboxylic acid;
1-Cyclopropyl-8-difluoromethoxy-7-(1S-methyl-1,2,3,4-tetrahydro-pyrrolo[1,2-a]pyrazin-7-yl)-4-oxo-1,4-dihydro-quinoline-3-carboxylic acid;
1-Cyclopropyl-8-difluoromethoxy-7-(1R-methyl-1,2,3,4-tetrahydro-pyrrolo[1,2-a]pyrazin-7-yl)-4-oxo-1,4-dihydro-quinoline-3-carboxylic acid;
1-Cyclopropyl-8-difluoromethoxy-7-[2S-(2-methylamino-propionyl)-1,2,3,4-tetrahydro-pyrrolo[1,2-a]pyrazin-7-yl]-4-oxo-1,4-dihydro-quinoline-3-carboxylic acid;
1-Cyclopropyl-6-fluoro-8-methoxy-7-(3S-methyl-1,2,3,4-tetrahydro-pyrrolo[1,2-a]pyrazin-7-yl)-4-oxo-1,4-dihydro-quinoline-3-carboxylic acid;
1-Cyclopropyl-6-fluoro-8-methoxy-7-(3R-methyl-1,2,3,4-tetrahydro-pyrrolo[1,2-a]pyrazin-7-yl)-4-oxo-1,4-dihydro-quinoline-3-carboxylic acid;
1-Cyclopropyl-7-(2,3S-dimethyl-1,2,3,4-tetrahydro-pyrrolo[1,2-a]pyrazin-7-yl)-6-fluoro-8-methoxy-4-oxo-1,4-dihydro-quinoline-3-carboxylic acid;
1-Cyclopropyl-7-(2,3R-dimethyl-1,2,3,4-tetrahydro-pyrrolo[1,2-a]pyrazin-7-yl)-6-fluoro-8-methoxy-4-oxo-1,4-dihydro-quinoline-3-carboxylic acid;
1-Cyclopropyl-6-fluoro-7-(3R-hydroxymethyl-2-methyl-1,2,3,4-tetrahydro-pyrrolo[1,2-a]pyrazin-7-yl)-8-methoxy-4-oxo-1,4-dihydro-quinoline-3-carboxylic acid;
1-Cyclopropyl-6-fluoro-8-methoxy-7-(1S-methyl-1,2,3,4-tetrahydro-pyrrolo[1,2-a]pyrazin-7-yl)-4-oxo-1,4-dihydro-quinoline-3-carboxylic acid;
1-Cyclopropyl-6-fluoro-8-methoxy-7-(1R-methyl-1,2,3,4-tetrahydro-pyrrolo[1,2-a]pyrazin-7-yl)-4-oxo-1,4-dihydro-quinoline-3-carboxylic acid;
1-Cyclopropyl-7-(1R,3S-dimethyl-1,2,3,4-tetrahydro-pyrrolo[1,2-a]pyrazin-7-yl)-6-fluoro-8-methoxy-4-oxo-1,4-dihydro-quinoline-3-carboxylic acid;
1-Cyclopropyl-6-fluoro-7-(3S-methyl-1,2,3,4-tetrahydro-pyrrolo[1,2-a]pyrazin-7-yl)-4-oxo-1,4-dihydro-quinoline-3-carboxylic acid;
1-Cyclopropyl-7-(2,3S-dimethyl-1,2,3,4-tetrahydro-pyrrolo[1,2-a]pyrazin-7-yl)-6-fluoro-4-oxo-1,4-dihydro-quinoline-3-carboxylic acid;
1-Cyclopropyl-6-fluoro-7-(1S-methyl-1,2,3,4-tetrahydro-pyrrolo[1,2-a]pyrazin-7-yl)-4-oxo-1,4-dihydro-quinoline-3-carboxylic acid;
1-Cyclopropyl-6-fluoro-7-(1R-methyl-1,2,3,4-tetrahydro-pyrrolo[1,2-a]pyrazin-7-yl)-4-oxo-1,4-dihydro-quinoline-3-carboxylic acid;
8-Fluoro-3S-methyl-9-(3S-methyl-1,2,3,4-tetrahydro-pyrrolo[1,2-a]pyrazin-7-yl)-6-oxo-2,3-dihydro-6H-1-oxa-3a-aza-phenalene-5-carboxylic acid;
8-Fluoro-3S-methyl-9-(1S-methyl-1,2,3,4-tetrahydro-pyrrolo[1,2-a]pyrazin-7-yl)-6-oxo-2,3-dihydro-6H-1-oxa-3a-aza-phenalene-5-carboxylic acid;
1-Cyclopropyl-6-fluoro-7-(3S-methyl-1,2,3,4-tetrahydro-pyrrolo[1,2-a]pyrazin-7-yl)-4-oxo-1,4-dihydro-[1,8]naphthyridine-3-carboxylic acid;
1-Cyclopropyl-6-fluoro-7-(1S-methyl-1,2,3,4-tetrahydro-pyrrolo[1,2-a]pyrazin-7-yl)-4-oxo-1,4-dihydro-[1,8]naphthyridine-3-carboxylic acid;
1-(2,4-Difluoro-phenyl)-6-fluoro-7-(3S-methyl-1,2,3,4-tetrahydro-pyrrolo[1,2-a]pyrazin-7-yl)-4-oxo-1,4-dihydro-[1,8]naphthyridine-3-carboxylic acid;
1-(2,4-Difluoro-phenyl)-6-fluoro-7-(1S-methyl-1,2,3,4-tetrahydro-pyrrolo[1,2-a]pyrazin-7-yl)-4-oxo-1,4-dihydro-[1,8]naphthyridine-3-carboxylic acid;
1-Cyclopropyl-8-difluoromethoxy-6-fluoro-7-(1S-methyl-1,2,3,4-tetrahydro-pyrrolo[1,2-a]pyrazin-7-yl)-4-oxo-1,4-dihydro-quinoline-3-carboxylic acid;
7-(2-Benzyl-1,2,3,4-tetrahydro-pyrrolo[1,2-a]pyrazin-7-yl)-1-(2,4-difluoro-phenyl)-6-fluoro-4-oxo-1,4-dihydro-[1,8]naphthyridine-3-carboxylic acid;
7-(2-Benzyl-1,2,3,4-tetrahydro-pyrrolo[1,2-a]pyrazin-7-yl)-1-cyclopropyl-6-fluoro-8-methoxy-4-oxo-1,4-dihydro-quinoline-3-carboxylic acid;
1-Cyclopropyl-6-fluoro-7-(1,2,3,4,6,8a-hexahydro-pyrrolo[1,2-a]pyrazin-7-yl)-8-methoxy-4-oxo-1,4-dihydro-quinoline-3-carboxylic acid;
8-Fluoro-9-(1,2,3,4,6,8a-hexahydro-pyrrolo[1,2-a]pyrazin-7-yl)-3S-methyl-6-oxo-2,3-dihydro-6H-1-oxa-3a-aza-phenalene-5-carboxylic acid;
1-Cyclopropyl-6-fluoro-8-methoxy-4-oxo-7-(1,2,3,4-tetrahydro-pyrrolo[1,2-a]pyrazin-7-yl)-1,4-dihydro-quinoline-3-carboxylic acid;
8-Fluoro-3S-methyl-6-oxo-9-(1,2,3,4-tetrahydro-pyrrolo[1,2-a]pyrazin-7-yl)-2,3-dihydro-6H-1-oxa-3a-aza-phenalene-5-carboxylic acid;
and optical isomers, diastereomers, enantiomers, pharmaceutically acceptable salts, hydrates, and prodrugs thereof.

11. A compound as in claim 4, selected from the group consisting of:
1-Cyclopropyl-8-difluoromethoxy-4-oxo-7-(1,2,3,4-tetrahydro-pyrrolo[1,2-a]pyrazin-7-yl)-1,4-dihydro-quinoline-3-carboxylic acid;
1-Cyclopropyl-6-fluoro-8-methoxy-7-(3S-methyl-1,2,3,4-tetrahydro-pyrrolo[1,2-a]pyrazin-7-yl)-4-oxo-1,4-dihydro-quinoline-3-carboxylic acid;
1-Cyclopropyl-6-fluoro-8-methoxy-7-(3R-methyl-1,2,3,4-tetrahydro-pyrrolo[1,2-a]pyrazin-7-yl)-4-oxo-1,4-dihydro-quinoline-3-carboxylic acid;
1-Cyclopropyl-7-(2,3S-dimethyl-1,2,3,4-tetrahydro-pyrrolo[1,2-a]pyrazin-7-yl)-6-fluoro-8-methoxy-4-oxo-1,4-dihydro-quinoline-3-carboxylic acid;
1-Cyclopropyl-6-fluoro-8-methoxy-7-(1S-methyl-1,2,3,4-tetrahydro-pyrrolo[1,2-a]pyrazin-7-yl)-4-oxo-1,4-dihydro-quinoline-3-carboxylic acid;
1-Cyclopropyl-6-fluoro-7-(3S-methyl-1,2,3,4-tetrahydro-pyrrolo[1,2-a]pyrazin-7-yl)-4-oxo-1,4-dihydro-[1,8]naphthyridine-3-carboxylic acid;
1-Cyclopropyl-6-fluoro-8-methoxy-4-oxo-7-(1,2,3,4-tetrahydro-pyrrolo[1,2-a]pyrazin-7-yl)-1,4-dihydro-quinoline-3-carboxylic acid;
and optical isomers, diastereomers, enantiomers, pharmaceutically acceptable salts, hydrates, and prodrugs thereof.

12. A pharmaceutical composition comprising a pharmaceutically acceptable carrier and a compound of claim 1.

13. A pharmaceutical composition made by mixing a compound of claim 1 and a pharmaceutically acceptable carrier.

14. A process for making a pharmaceutical composition comprising mixing a compound of claim 1 and a pharmaceutically acceptable carrier.

15. A method of treating a subject having a condition caused by or contributed to by bacterial infection, comprising administering to a subject in need thereof a therapeutically effective amount of the compound as in claim 1.

16. A method of preventing a subject from suffering from a condition caused by or contributed to by bacterial infection, comprising administering to a subject in need thereof a prophylactically effective dose of a compound as in claim 1.

17. The use of a compound as in claim 1 for the preparation of a medicament for treating or preventing a condition caused by or contributed to by bacterial infection, in a subject in need thereof.
